(19) United States
(12) Patent Application Publication
(10) Pub. No.: US 2026/0117315 A1
(43) Pub. Date: Apr. 30, 2026

(54) KEY PREDOMINANT SPECIES OF GUT BACTERIA COLONIZING FARM-EXPOSED INFANTS

(71) Applicant: Wisconsin Alumni Research Foundation, Madison, WI (US)

(72) Inventors: Irene Ong, Fitchburg, WI (US); Christine Seroogy, Madison, WI (US); James Gern, Middleton, WI (US); Deborah Chasman, Madison, WI (US)

(73) Assignee: Wisconsin Alumni Research Foundation, Madison, WI (US)

(21) Appl. No.: 19/345,576

(22) Filed: Sep. 30, 2025

Related U.S. Application Data
(63) Continuation of application No. 17/935,863, filed on Sep. 27, 2022, now abandoned.
(60) Provisional application No. 63/248,940, filed on Sep. 27, 2021.

(51) Int. Cl. C12Q 1/689; C12Q 1/6869

(52) U.S. Cl. CPC C12Q 1/689; C12Q 1/6869; C12Q 2600/118

(57) ABSTRACT

A method to detect immune health status in a human infant or child, and compositions and methods to improve health status in a human fetus, infant or child, as well as compositions and methods useful to improve immune health status, are provided.

### CROSS-REFERENCE TO RELATED APPLICATIONS

This application is a continuation of U.S. application Ser. No. 17/935,863, filed Sep. 27, 2022, which claims the benefit of the filing date of U.S. application No. 63/248,940, filed on Sep. 27, 2021, the disclosure of which is incorporated by reference herein.

### BACKGROUND

Allergic diseases, including asthma, are initiated in early life by the development of sensitization to environmental allergens. Once allergic sensitization is established, treatment is primarily focused on management of symptoms. Children living in farming households have lower prevalence of these diseases (Jatzlauk et al., 2017; Stein et al., 2016; Haahtela et al., 2015; Tantoco et al., 2018; Alfvén et al., 2006; Ege et al., 2011; Riedler et al., 2001; Von Ehrenstein et al., 2000), and even lower allergic disease prevalence is commensurate with longer term and earlier life exposures (Riedler et al., 2001), especially in more traditional agrarian communities, such as the TA (Tantoco et al., 2018; Stein et al., 2016). The reasons for this health disparity are not secondary to genetic differences, but in large part are attributable to environmental pressures that are believed to be modifiable, if the protective environmental exposures and their interaction with the developing immune system can be accurately defined (Stein et al., 2016; von Mutius and Vercelli, 2010).

### SUMMARY

There is a growing understanding that microbes in the environment interact with the immune cells in our bodies in many ways including through our gut and that there is a small window in the first few years of life for intervention. That is, diverse bacteria in early life may be necessary for the development of a normal immune system and there are increasing numbers of the population who live in industrialized settings who have gut microbiomes that are lacking protective species as beginning at birth. For example, differences in lifestyle, which includes diet, between non-farm, farm and Amish children, the latter of which are referred to herein as traditional agrarian (TA) (FIG. 1) lead to different levels of animal and thus microbial exposures experienced by these children growing up on farm or non-farm environments. As disclosed herein, the level of farm exposure correlates with the degree of protection, e.g., people following traditional agrarian lifestyles experience lower rates of asthma compared to more industrialized farming communities. Thus, microbial exposures in early life may lay the foundation for the development of a healthy immune system.

The depletion of good microbes or the presence of bad microbes in the gut at certain times, and in particular microbes established in early life, can influence development of necrotizing enterocolitis and irritable bowel disease (IBD), which can in turn increase the prevalence for the development of early onset colon cancer. Herein it is shown that diet and the environment in which an individual lives (lifestyle) influence the relative abundance of good and bad microorganisms that thrive in the gut. The presence of Bifidobacterium species in the maternal vagina and infant gut is an evolutionary trait that selects for these organisms to be primary colonizers of the newborn intestinal tract. Their ability to utilize human milk oligosaccharides and the fact that human milk IgA antibodies bind to Bifidobacterium, fosters their establishment as core health-promoting organisms throughout life. A reduction in their abundance in infants has been associated with the prevalence of obesity, diabetes, metabolic disorder, cancer and other causes of mortality later in life.

In particular, the Wisconsin Infant Study Cohort (WISC) birth cohort aimed at characterizing the impact of early life farming exposures on immune development, respiratory health, and allergic diseases (Seroogy et al., 2019). Study participants were recruited for three arms: TA, modern dairy farming, and rural non-farming study group. As disclosed herein, stool samples collected from study infants at 2 months of age underwent shotgun metagenomic sequencing to perform a comparative analysis between the study arms. The findings showed a significant increase in Bifidobacteria in the Wisconsin Farm Study group (WFS, composed of TA infants) as compared to the other study groups (modem, non-TA dairy farming, and rural, non-TA and non-farming). Specifically, the microbiota from the WFS infant stool samples were characterized by a striking dominance of Bifidobacterium longum. While Bifidobacterium species were high in breastfeeding children across all three groups (e.g., compared to formula fed infants), the relative abundance of Bifidobacterium longum was higher in the WFS group, while other Bifidobacterium species (breve, bifidum) were found in higher abundance in the non-TA study groups. Furthermore, the WFS microbiota harbored unique gene families, including several that are specific to previously annotated strains of Bifidobacterium longum subsp. infantis. As shown in FIG. 7, between newborn to 6 months of age, infants with microbiomes comprising predominantly of Bifidobacterium species (as associated with agrarian lifestyle, which is associated with better health), whereas those with higher composition of diverse microbiomes especially of microbes other than Bifidobacterium (e.g. see formula panel where there are high streptococcus, staphylococcus, E. coli, and other non-Bifidobacterium and non-Lactobacillus), during this time period, are associated with WISC farm and non-farm, which have increased likelihood of developing immune-related diseases such as allergies. Specifically, Bifidobacterium longum subsp. infantis in particular, provides decreased risk of allergies (viz-a-viz TA vs WISC farm and non-farm allergy prevalence). In general, a higher abundance of Bifidobacterium species is beneficial, e.g., 80% vs 25%, because these microbes may provide beneficial metabolites and/or prevent colonization by pathogenic bacteria or other microbes (e.g., E. coli, Streptococcus, and/or Staphylococcus or fungi), while a lower Bifidobacterium percentage may not confer these benefits, e.g., there may be a higher percentage of other microbes (e.g., higher non-Bifidobacterium diversity) including but not limited to pathogenic bacteria such as E. coli, Streptococcus, and Staphylococcus, which leads to increased risk of allergies and other diseases. For example, the over-abundance of specifically B. longum subsp. infantis (from 60% up to 90% in TA infants) and/or a total percentage of B. longum subsp. infantis, B. bifidum and B. breve as >60% may promote and/or may be indicative of immune health.

Thus, bacteria and molecules that enhance the prevalence and/or activity of certain bacteria in the gut microbiome, e.g., human milk oligosaccharides, antibodies, e.g., from breastmilk, as well as other molecules, such as mucin binding proteins or peptides, e.g., produced by B. longum or B. infantis, oligosaccharides, or glycans, molecules that increase mucin production, or exosomes produced by bacteria including Bifidobacterium, e.g., B. longum, or any molecules that reduce leaky gut or enhance bifidobacterial adhesion and survival in the GI tract, thereby enhancing growth and higher abundance of Bifidobacterium, may be employed in compositions, e.g., as a prebiotic (substance or food ingredient that promotes the growth of beneficial microbes in the gut) or probiotic (culture of a specific microbe or combination of microbes) supplement that can be included with or added to formula or ingested prepregnancy by women who are trying to become pregnant, by expectant (pregnant) or breastfeeding mothers to promote and/or increase the Bifidobacterium longum infantis abundance in maternal or infant gut microbiome, thereby promoting healthy development including protections from immune-related diseases such as allergies or other diseases. Comparative functional analysis of Bifidobacterium also identified that Bifidobacterium can produce more indole-3-lactic acid, folic acid and riboflavin (vitamin B2) among other metabolites. Some of these metabolites, including indole-3-lactic acid have shown immunoregulatory effects, including suppression of TH2 and TH17 cytokines and induction of interferon beta. In one embodiment, the composition is a liquid comprising an amount of Bifidobacterium longum infantis such as a strain obtained from a WFS infant, optionally with one or more prebiotic and/or probiotics, or immunoglobulin A (IgA) antibodies, that select for prevalence of Bifidobacterium longum infanti, and/or that enhance the activity, e.g., colonization or enzyme activity, of Bifidobacterium longum infantis in the gut of a newborn or child. In one embodiment, the composition is ingested by prepregnancy by women who are trying to become pregnant, by expectant (pregnant) or a lactating mother (e.g., human) or exclusively breastfed infant, or by an infant via formula. In one embodiment, the composition comprises microbes with the functional capacities of Bifidobacterium longum infantis, e.g., to metabolize human milk oligosaccharides by transforming the microbes with genes that encode human milk oligosaccharide metabolizing enzymes and/or other genes that promote health, e.g., genes for biosynthesis of folic acid, riboflavin, p-Cresol sulfate, tryptophan and/or other metabolites in the tryptophan pathway.

The disclosure provides a method to detect immune health status and potentially for providing as prebiotic or probiotic for treatment of metabolism, immune or neurodegenerative related diseases (e.g., autism) in a human infant (e.g., up to 6 to 9 months or 1 year of age) or child (including toddlers from 1 to 3 years of age and adolescents up to 18 years of age). The method includes providing a physiological sample, e.g., a stool sample, from a human infant or child and determining in the sample i) the relative abundance of bacteria including two or more of Bacteroides, Bifidobacterium, or Blautia, ii) the relative abundance of bacteria including two or more of Bifidobacterium bifidum, Bifidobacterium breve, Bifidobacterium longum, or Bifidobacterium pseudocatenulatum, or iii) the relative abundance or expression of one, two or more of Blon_0915, Blon_2171, Blon_2173, Blon_2334, galT Blon_2172, Blon_2177, Blon_0625, Blon_0244, Blon_0248; Blon_0426, ureF Blon_0113, ureC Blon_0111, ureE Blon_0112 BLIJ_0113, Blon_0642, Blon_2336, Blon_2344, or Blon_0650 or one, two or more of Blon_0915, Blon_2177, Blon_0625, Blon_0244, Blon_0248; Blon_0426, ureF Blon_0113, ureC Blon_0111, ureE Blon_0112 BLJ_0113, Blon_0642, Blon_2336, Blon_2344, or Blon_0650. In one embodiment, a relative abundance of Bacteroides of >10%, of Bifidobacterium of <60% or of Blautia of >10% is indicative of an infant or child at increased risk of allergies or other diseases, e.g., IBD, type 2 diabetes, or obesity. In one embodiment, a relative abundance of Bifidobacterium of <60% is indicative of an infant or child at increased risk of allergies or other diseases, e.g., IBD, type 2 diabetes, or obesity. In one embodiment, a relative abundance of Bacteroides of >10%, of Bifidobacterium of <60% and of Blautia of >10% is indicative of an infant or child at increased risk of allergies, e.g., IBD, type 2 diabetes, or obesity. In one embodiment, a relative abundance of Bacteroides of <10%, of Bifidobacterium of >60% or of Blautia of <10% is indicative of an infant or child at decreased risk of allergies or other diseases, e.g., IBD, type 2 diabetes, or obesity. In one embodiment, a relative abundance of Bifidobacterium of >60 is indicative of an infant or child at decreased risk of allergies or other diseases, e.g., IBD, type 2 diabetes, or obesity. In one embodiment, a relative abundance of Bacteroides of <10%, of Bifidobacterium of >60% and of Blautia of <10% is indicative of an infant or child at decreased risk of allergies or other diseases, e.g., IBD, type 2 diabetes, or obesity. In one embodiment, a relative abundance of Bifidobacterium bifidum of 5% or greater or 10% or less, Bifidobacterium breve of 2% or greater or 25% or less, Bifidobacterium longum of 25% or greater, or of Bifidobacterium pseudocatenulatum of less than 2% is indicative of immune health in the infant or child. In one embodiment, a relative abundance of Bifidobacterium bifidum of 5% or greater or 10% or less, Bifidobacterium breve of 2% or greater or 25% or less, Bifidobacterium longum of 25% or greater, or of Bifidobacterium pseudocatenulatum of less than 2% is indicative of immune health in the infant or child. In one embodiment, a relative abundance of Bifidobacterium bifidum of 5% or greater or 10% or less, Bifidobacterium breve of 2% or greater or 25% or less, Bifidobacterium longum of 25% or greater, and of Bifidobacterium pseudocatenulatum of less than 2% is indicative of immune health in the infant or child. In one embodiment, a relative abundance of Bifidobacterium bifidum of 5% or greater, Bifidobacterium breve of 20% or less, Bifidobacterium longum of 50% or greater, or of Bifidobacterium pseudocatenulatum of less than 2% is indicative of immune health in the infant or child. In one embodiment, a relative abundance of Bifidobacterium bifidum of 5% or greater, Bifidobacterium breve of 20% or less, Bifidobacterium longum of 50% or greater, and of Bifidobacterium pseudocatenulatum of less than 2% is indicative of immune health in the infant or child. In one embodiment, a relative abundance of Bifidobacterium bifidum of less than 5%, Bifidobacterium breve of greater than 20%, Bifidobacterium longum of less than 50%, or of Bifidobacterium pseudocatenulatum of greater than 2% is indicative of impaired immune health in the infant or child. In one embodiment, an increase in the relative abundance of expression of one or more of Blon_0915, Blon_2171, Blon_2173, Blon_2334, galT Blon_2172, Blon_0244, Blon_0248; Blon_0426, ureF Blon_0113, ureC Blon_0111, ureE Blon_0112 BLIJ_0113, Blon_0642, Blon_2336, Blon_2344, or Blon_0650 is indicative of immune health in the infant or child. In one embodiment, the sample is from a newborn. In one embodiment, the sample is from a newborn to 3 month old. In one embodiment, the sample is from a 3 month old to a 6 month old. In one embodiment, the sample is from an infant treated with a drug. In one embodiment, the drug is an antibiotic. In one embodiment, the prebiotic and/or probiotic is administered before the antibiotic, e.g., 1, 2, 3, 4 5 or 6 hours or more, apart. In one embodiment, the infant or child has necrotizing enterocolitis. In one embodiment, the method includes administering to the infant or child a prebiotic or a probiotic. In one embodiment, the prebiotic or probiotic comprises one or more bacteria, one or more antibodies, or one or more molecules that enhance the relative abundance of Bifidobacterium longum. In one embodiment, the relative abundance of Bifidobacterium longum infantis is enhanced. In one embodiment, the abundance is enhanced to greater than 60%, 70%, 80% or 90%. In one embodiment, the sample is analyzed using a nucleic acid amplification reaction. In one embodiment, the sample is analyzed using genome sequencing. In some embodiments, the sample is analyzed using bioluminescence or antibodies with fluorophores, or tags such as a nucleic acid barcode or magnetic beads.

In one embodiment, a relative abundance of Bacteroides of >8%, of Bifidobacterium of <65% or of Blautia of >2% is indicative of an infant or child at increased risk of allergies. In one embodiment, a relative abundance of Bacteroides of >10%, of Bifidobacterium of <60% and of Blautia of >10% is indicative of an infant or child at increased risk of allergies, BD, type 2 diabetes, or obesity. In one embodiment, a relative abundance of Bacteroides of >8%, of Bifidobacterium of <65% and of Blautia of >2% is indicative of an infant or child at increased risk of allergies, I BD, type 2 diabetes, or obesity. In one embodiment, a relative abundance of Bacteroides of <10%, of Bifidobacterium of >60% or of Blautia of <10% is indicative of an infant or child at decreased risk of allergies or Bacteroides of <10%, of Bifidobacterium of >65% or of Blautia of <2% is indicative of an infant or child at decreased risk of allergies, I BD, type 2 diabetes, or obesity. In one embodiment, a relative abundance of Bacteroides of <10%, of Bifidobacterium of >60% and of Blautia of <10% is indicative of an infant or child at decreased risk of allergies, I BD, type 2 diabetes, or obesity. In one embodiment, a relative abundance of Bacteroides of <10%, of Bifidobacterium of >65% or of Blautia of <2% is indicative of an infant or child at decreased risk of allergies, IBD, type 2 diabetes, or obesity. In one embodiment, a relative abundance of Bifidobacterium bifidum of 5% to 10%, Bifidobacterium breve of 2% to 25%, Bifidobacterium longum of 25% or greater, or of Bifidobacterium pseudocatenulatum of less than 2% is indicative of immune health in the infant or child. In one embodiment, a relative abundance of Bifidobacterium bifidum of 10% or less, Bifidobacterium breve of 25% or less, Bifidobacterium longum of 25% or greater, or of Bifidobacterium pseudocatenulatum of less than 2% is indicative of immune health in the infant or child or of Bifidobacterium breve of 15% or less, Bifidobacterium longum of 65% or greater, or of Bifidobacterium pseudocatenulatum of less than 3% is indicative of immune health in the infant or child. In one embodiment, a relative abundance of Bifidobacterium bifidum of 10% or less, Bifidobacterium breve of 25% or less, Bifidobacterium longum of 25% or greater, and of Bifidobacterium pseudocatenulatum of less than 2% is indicative of immune health in the infant or child or of Bifidobacterium breve of 15% or less, Bifidobacterium longum of 65% or greater, and of Bifidobacterium pseudocatenulatum of less than 3% is indicative of immune health in the infant or child. In one embodiment, a relative abundance of Bifidobacterium bifidum of 5% or greater, Bifidobacterium breve of 20% or less, Bifidobacterium longum of 50% or greater, or of Bifidobacterium pseudocatenulatum of less than 2% is indicative of immune health in the infant or child. In one embodiment, a relative abundance of Bifidobacterium bifidum of 5% or greater, Bifidobacterium breve of 20% or less, Bifidobacterium longum of 50% or greater, and of Bifidobacterium pseudocatenulatum of less than 2% is indicative of immune health in the infant or child. In one embodiment, a relative abundance of Bifidobacterium bifidum of less than 5%, Bifidobacterium breve of greater than 20%, Bifidobacterium longum of less than 50%, or of Bifidobacterium pseudocatenulatum of greater than 2% is indicative of impaired immune health in the infant or child or of Bifidobacterium breve of greater than 15%, Bifidobacterium longum of less than 30%, or of Bifidobacterium pseudocatenulatum of greater than 3% is indicative of impaired immune health in the infant or child.

In one embodiment, a method to identify a human infant or child at higher risk of developing allergies is provided. The method includes providing a stool sample from a human infant or child; and determining in the sample i) the relative abundance of bacteria including two or more of Bacteroides, Bifidobacterium, or Blautia, ii) the relative abundance of bacteria including two or more of Bifidobacterium bifidum, Bifidobacterium breve, Bifidobacterium longum, or Bifidobacterium pseudocatenulatum, or iii) the relative abundance or expression of one, two or more of Blon_0915, Blon_2177, Blon_0625, Blon_0244, Blon_0248; Blon_0426, ureF, Blon_0113, ureC Blon_0111, ureE Blon_0112 BLIJ_0113, Blon_0642, Blon_2336, Blon_2344, or Blon_0650.

Other organisms that may be detected include but are not limited to Parabacteroides merdae or Bacteroides stercoris (associated with WFS; glmnet features and others), Bacteroides thetaiotaomicron (associated with WISC), Parabacteroides and Bacteroides identified by screening for (adult) gut microbes that could attenuate epithelial cell line IL-8 response to LPS (Hiippala et al., 2020) or Collinsella aerofaciens (higher in WFS) (Collinsella species were previously associated with higher Bifidobacterium in infant gut (Milani et al. 2017)), and those of higher abundance in WISC (based on glmnet features), e.g., Veillonella or Cutibacterium.

Also provided are products for consumption, e.g., a composition comprising one or more agents such as a prebiotic(s) and/or probiotic(s), for example, to promote infant health and/or long term immune health, thereby decreasing the incidence of aberrant immune responses that are observed in autoimmune diseases such as allergies, inflammatory bowel disease (IBD), type 2 diabetes, metabolic disease, such as obesity, and neurodegenerative diseases such as ADHD, autism and the like. The compositions may be useful to stimulate an anti-inflammatory state in a pregnant female, infant, toddler or child, e.g., under the age of 5 years old. An anti-inflammatory state may also be useful to prevent or inhibit cancer. In one embodiment, the composition may include one or more B vitamins, one or more short chain fatty acids, linoleic acid, linolenic acid, tryptophan, one or more tryptophan metabolites such as p-cresol, oxoglutaric acid, indole-3-methylacetate, or one or more hydroxyoctadecadienoic acids, or combinations thereof, or isolated bacteria such as Bifidobacteria (e.g., B. infantis, B. longum, B. breve, and/or B. bifidum, or a combination thereof), or bacteria genetically modified to overexpress human breast milk oligosaccharide metabolizing enzymes, or modified with, for example, galT, ureF, ureC and/or ureE genes, e.g., from Bifidobacterium longum subsp. infantis. (B. infantis), B. longum, B. breve and/or B. bifidum), that may be used as probiotics along with breastmilk or sugars present in breastmilk such as 2-fucosylactose, sialylated lactose, lacto-N-biose, galacto-N-biose, and the like. Furthermore, some of the exopolypeptides and metabolites produced by these Bifidobacteria microbes modulate immune responses and neural growth, e.g., Bifidobacteria-specific surface exopolysaccharide (EPS), which may provide a protective biofilm against pathogens, an indole such as indolelactic acid: products of tryptophan degradation, which promote anti-inflammation and immune tolerance in gut epithelial cells and immune cells via aryl hydrocarbon receptor (AHR) signaling pathway, gamma-aminobutyrate (GABA), and acetate, a short chain fatty acid (SCFA) which stimulates 5-hydroxytryptamine (serotonin, important neurotransmitter) production by gut enterochromaffin cells. Acetate is produced by Bifidobacteria. In one embodiment, the composition is breast milk formula (baby or infant formula) (e.g., powder or liquid) supplemented with the agents, e.g., prebiotic(s) and/or probiotic(s) disclosed herein. For example, molecules that are more prevalent in in TA and/or farm 2-month-old infant stool compared to WISC (see FIGS. 27 and 29) may be employed in the compositions, molecules including but not limited to, folic acid, riboflavin, aromatic amino acids (tryptophan, tyrosine, phenylalanine), adenine, 4-hydroxyphenyllactic acid (4-OH-PLA), pnenyllactic acid (PLA), and indole-3-lactic acid (ILA) (aryllactic acids) which are ligands for hydroxycarboxylic acid receptors, which play important roles in maintaining energy and immune homeostasis (Ahmed et al Front. Endocrinol. 2011), gamma-glutamylmethionine, cysteine, a sulfur containing amino acid, which exerts functions through metabolites such as S-adenosylmethionine (SAM), N-acyl-DL-glutamic acid and the like. FIG. 29 shows that indole-3-methyl-acetate was found to be lower in 2-month-old infant stool metabolites from from farm compared to non-farm infants and 1-year old infant plasma metabolites measured identified oxoglutaric acid, and p-cresol sulfate as significantly higher in farm infants compared to non-farm infants. Exopolypeptides from Bifidobacterium breve have been shown to prevent maturation of dendritic cells and activation of antigen specific CD4+ T cells responses to B. breve in mice, suggesting it may be important for immune evasion of adaptive immunity and contribute to host-microbe mutualism

Further provided are methods of using the compositions, e.g., to prevent, inhibit or treat an inflammatory, metabolic, gastrointestinal, or neurodegenerative conditions in a mammal in need thereof, e.g., to enhance an anti-inflammatory response to one or more antigens in a mammal, or to prevent, inhibit or treat one or more symptoms in a mammal having or at risk of an allergic disease, e.g., asthma, eczema, or other autoimmune diseases, or metabolic, gastrointestinal, or neurodegenerative diseases.

### BRIEF DESCRIPTION OF FIGURES

FIG. 1A. Early life environment is associated with decreased prevalence of allergic diseases. TA children have a lower prevalence of allergic disease. For example, TA children have a 10 times lower eczema prevalence. Children who moved to farms after the age of 5 did not seem to gain protective effects experienced by those who lived on farms from birth.

FIG. 1B. Early life farm exposures protect against development of allergic diseases and asthma. Marshfield Epidemiological Study Area (MESA). Adapted from Ludka-Gaulke et al. JACI 2018.

FIG. 1C. Eczema prevalence is lOX lower in WI TA children and early life farm exposures protective. The Wisconsin Plain Community Project survey (Tantoco et al., Ann Allergy Asthma Immunol 2018, n=2781 children). Wisconsin Infant Study Cohort (Seroogy et al., Respir Res 2019, Steiman et al., J Allergy Clin Immunol 2020).

FIG. 2A. Wisconsin Infant Study Cohort (WISC) and Wisconsin Farm Study (WFS). The Wisconsin Infant Study Cohort (WISC) and Wisconsin Farm Study are prospective birth cohort studies that aim to identify molecular contributors of farm exposures on development of asthma and childhood respiratory illness.

FIG. 2B. Metagenomics sequencing.

FIG. 2C. 116 metagenomics profiles of stool from two-month-old infants.

FIG. 3. Infant gut microbiome is associated with diet and farm exposure. Association between subject characteristics and alpha diversity metrics. Tests are either Kruskal-Wallis (three categorical outcomes: diet.at.02, farm) or Mann-Whitney (binary outcomes: WFS_vs_WISC, curr_breastmilk.at.02, exclusive_breastmilk.at.02). *p<0.05 after correcting for multiple hypothesis tests by Benjamini-Hochberg 25 procedure. “Farm” subsets are results of performing farm group comparisons restricted to “currently breastfeeding” or “exclusively breastfeeding” participants only.

FIG. 4. Association between subject characteristics and beta diversity metrics. *PERMANOVA p<0.05 after 6 correcting for multiple hypothesis tests by Benjamini-Hochberg procedure. “Farm” subsets are results of performing farm group comparisons restricted to “currently breastfeeding” or “exclusively breastfeeding” participants only.

FIG. 5. Relative abundance of top genera by child diet at sample collection (all infants). Breastfeeding: exclusive breastfeeding; formula: exclusively formula feeding; both: both breastfeeding and formula feeding. Genera were included with relative abundance of at least 1% in at least 10% of study samples.

FIG. 6. Average relative abundance of top genera (all infants). Genera were included with relative abundance of at least 1% in at least 10% of study samples.

FIG. 7. Relative abundance of Bifidobacterium species in microbiota of study participants. Grey (“Other”): non-Bifidobacterium species.

FIG. 8. Relative abundance of Bifidobacterium species, aggregated by farm group. Grey (“Other”): non-Bifidobacterium species.

FIG. 9. Comparison of infant microbiome structure across US.

FIG. 10. Comparison of Bifidobacterium longum gene family representation in study samples and reference genomes. A pangenome analysis was conducted to survey which Bifidobacterium genes that were present in each sample and to compare them to reference genomes. Each row in this heatmap represents a UniRef90 gene family that was found in at least one publicly available reference genome for Bifidobacterium longum. Each column is either a study sample or a reference genome. Dark gray indicates presence and light gray indicates absence of the gene. The first bottom annotation indicates reference genomes, TA, farm, and nonfarm. A diet annotation is included with exclusive breastfeeding indicated. For reference genomes, subspecies annotation, if available, are shown in the bottom row annotation. Subspecies infantis, suis, and longum are shown. Using hierarchical clustering to compare the gene family representation in the study samples with reference genomes, this heatmap shows that TA samples clustered next to known infantis strains (boxes).

FIG. 11. Machine learning performance on discriminating TA from non-TA, exclusively breastfeeding infants only. Value is area under the precision-recall curve (PR-AUC).

FIG. 12. Cladogram of differentially abundant microbial taxa as assessed by LEfSE. Selected with p<0.05 and LDA score >2.

FIG. 13. Union of top 25 features selected by each of elastic net (glmnet) and random forest (ranger). Top 25 features ranked by median importance from 100 models. Intensity indicates the sign of the coefficient (positive for TA, negative for non-TA). Values are centered log-ratio relative abundances.

FIG. 14. Bifidobacterium longum subsp. infantis functional capacity to produce folic acid. MaAslin2 software was used to perform linear hypothesis tests on each pathway, to determine whether the pathway was differentially abundant between TA and non-TA. Bifidobacterium is protective, provides nutrients, and metabolites necessary for growth and development, whereas most other bacteria cause inflammation. The total folate transformations pathway is shown here with contributions broken down by taxon. Boxplots represent TA, farm, and non-farm. Most of the contribution is from different strains of B. longum.

FIG. 15. Bifidobacterium longum subsp. infantis functional capacity to produce tetrahydrofolate. TA and WISC infants have differential functional capacity to produce tetrahydrofolate.

FIG. 16. Bifidobacterium longum subsp. infantis functional capacity to produce flavin. TA and WISC infants have differential functional capacity to produce flavin.

FIG. 17. Association between subject characteristics and alpha diversity metrics. Tests are either Kruskal-Wallis (categorical outcomes) or Mann-Whitney (binary outcomes). *p<0.05 after correcting for multiple hypothesis tests by Benjamini-Hochberg procedure. “Farm” subsets are results of performing farm group comparisons restricted to “currently breastfeeding” or “exclusively breastfeeding” participants only.

FIG. 18. Comparison of Bifidobacterium longum gene family representation in study samples and reference genomes. Each row is a UniRef90 gene family that was found in at least one publicly available reference genome for Bifidobacterium longum. Each column is either a study sample or a reference genome, as indicated in the second from bottom column annotation. For reference genomes, subspecies annotation (if available) is given in the bottom column annotation.

FIG. 19. Top 25 features selected by elastic net (glmnet). Top: top 25 features ranked by median importance from 100 models. Shading indicates the sign of the coefficient (positive for TA, negative for non-TA). Bottom: same features, visualized as heatmap. Values are centered log-ratio relative abundances.

FIG. 20. Microbial community structure varies with farm group and diet. Beta diversity from species level features was computed using the Bray distance, and the samples were clustered with Dirichlet Multinomial Mixtures to identify latent structure. The Beta diversity plot on the left is annotated by the DMM cluster assignment, with cluster 1 in red, cluster 2 in blue and cluster 3 in green. The plot in the middle uses the same coordinates but is labeled by farm group. TA in squares, Farm in triangles, Nonfarm in circles. The plot on the right is annotated by the infant's diet at the time the sample was collected. Exclusively breastfed infants are stars, exclusively formula-fed infants in circles, and those with mixed diet of formula and breastfeeding in diamonds.

FIG. 21. Breastfeeding infant gut microbiome is dominated by Bifidobacterium. The stacked plots show the relative abundance of top genera aggregated by farm group and diet. The bars on the left show the exclusively breastfeeding infants, in the middle those with mixed diet, and on the right the exclusively formula-fed.

FIG. 22. Non-TA infants have more diverse microbiomes at species level.

FIG. 23. Pangenome files were obtained from the PanPhlan authors for B. longum, B. breve, and B. bifidum.

FIG. 24. WISC/WFS HMO profiles. Values are log 10(CPM+1). Top annotation is gene cluster, which is based on the organization of the genes on the B. infantis genome. Most of these genes are highly prevalent among TA samples and not among WISC samples. LoCascio reports that H5 is found commonly in other B. longum strains, so it is not surprising to see that it is prevalent in Farm and Nonfarm as well.

FIG. 25. Differential functional capacities.

FIG. 26. Machine learning models trained on stool metagenomics profiles can distinguish TA from non-TA.

FIG. 27. Correlated module of 2mo stool microbial pathway capacity and measured metabolites that are associated with Bifidobacterium longum-dominated microbiome and TA status. Partial correlations between microbial pathways and stool metabolites. * adjusted p<0.05.

FIG. 28. Correlated module of 2mo stool microbial pathway capacity and measured lipids that are associated with Bifidobacterium longum-dominated microbiome and TA status. Partial correlations between microbial pathways and stool.

FIG. 29. Farm status vs. selected tryptophan pathway metabolites (MW or KW test). Top row is Metabolon data from PLASMA12 and STOOL02. Bottom Row is STOOL02.

FIG. 30. Farm score vs tryptophan metabolites. Farm score is a function of number and frequency of farm animal exposures. Datasets: PLASMA00: data includes WFS and WISC; PLASMA12: either WFS and WISC or Metabolon (WISC only); and STOOL02: either WFS and WISC or Metabolon (WISC only). Rank (spearman) correlation of metabolite level to farm score (based on maternal and child farm exposures). No adjustment by sex or diet. Y axis is uncorrected p<0.05 positive correlation of metabolite to farm exposure; a negative correlation of metabolite to farm exposure.

FIG. 31. Microbiome-immune partial least squares (PLS) regression

FIG. 32. Mixed effects model.

FIG. 33. Principal components analysis (PCA) on STOOL02 metabolomics, lipidomics. Control samples in gray.

FIG. 34. Microbe-metabolomics module in network form (edges for significant partial Kendall correlations). This map shows connections between pathway (squares) and metabolites (circles). The ones with a wider outline around the circles and squares indicate they are higher in TA.

### DETAILED DESCRIPTION

One of the health-promoting attributes of human breast milk is to provide substrates for the developing gut microbiome. The loss of Bifidobacterium species from the infant gut microbiome, particularly Bifidobacterium longum infantis, in the first 3 months of life has been associated with a variety of negative health consequences including increased risk for allergic and other diseases. A recent report profiling infant gut microbial composition in the United States showed an overall low abundance (<50% on average) of Bifidobacterium genus in infants during the first 3 months of life. Thus, there is a need to identify dietary interventions to safely improve the altered infant gut microbiome. Human milk oligosaccharides (HMOs) present in human breast milk are one known substrate for promoting Bifidobacterium species, e.g., utilization of host derived glycans.

Studies have been steadily converging on the hypothesis that a major environmental contributor to immune development actually comes from within: the gut microbiome. Within the first few months of life, before the introduction of solid food, a microbiome dominated by only a few crucial taxa, including genus Bifidobacterium, has been associated with protection against asthma and other diseases later in life (Fujimura et al., 2016; Stokholm et al., 2018; Arrieta et al., 2015). However, which particular Bifidobacterium species and the composition of each species that contribute to lower prevalence has not been fully characterized. Bifidobacterium longum subspecies efficiently metabolize human milk oligosaccharides (HMOs); in particular, subsp. infantis has a contingent of unique genes for HMO metabolism compared to other subspecies (LoCascio et al., 2010) and have the capacity, e.g., genes to produce aromatic amino acids, aryllactic acids, sulfur amino acids, exopolysaccharides, and the like. Cohort studies have identified greater prevalence of infantis in traditional farming communities compared to communities that follow Western lifestyles (Seppo et al., 2021; Davis et al., 2017).

As disclosed herein below, Wisconsin TA (n=2,879) have a low rate (2.4%) of allergic diseases. Metagenomic sequencing was used to study the gut microbiomes of Wisconsin farm, non-farm and TA infants. Surprisingly, the predominant strain comprising ˜60-90% of the bacterial composition of the TA children's gut consists of one species: Bifidobacterium longum infantis. This bacteria co-evolved and so may have enhanced properties for breaking down human milk oligosaccharides, regulating metabolism, immune cells, neural, gastrointestinal and other cells in a human infant and other properties such as anti-viral properties.

In particular, gene profiling and metabolic potential bacterial colonies present in the gut microbiome of the infants were analyzed. Strains of bacteria isolated from the gut microbiome in the first two months of life in infants were isolated, e.g., strains of Bifidobacterium from infants having an increased prevalence of those strains. Those strains may be useful in a product to enhance immune health or prevent or lower the incidence of allergies or other diseases, e.g., the product may be used in newborns, children, adults, prepregnancy, and during pregnancy (expectant moms). For example, newborn stool may be analyzed to profile the microbiome through, for example, gene sequencing or nucleic acid amplification of specific genes, to characterize the potential immune health of the child and/or to identify deficiencies in the microbiome.

The postnatal, early-life developmental window is a critical time for establishing host-microbe interactions as the colonization by appropriate gastrointestinal microbes lay the foundation for the future health and well-being of the infant. Colonization by pioneer microbes shortly after birth, and the maintenance of this population, shapes the microbial community which in turn impacts numerous host physiological processes which can lead to a variety of negative consequences for host health including a predisposition to allergic disease or other diseases. Compositions, Routes of Administration, Dosages and Dosage Forms Provided herein are compositions that include but are not limited to one or more agents such as B vitamins, short chain fatty acids, linoleic caid, linolenic acid, tryptophan, tryptophan metabolites, and other metabolites such as folate or folic acid, aromatic amino acids (tryptophan, tyrosine, phenylalanine), tryptophan catabolites, aryllactic acids (4-OH-PLA, indole-3-lactic acid), GABA, SAM, sulfur amino acids (cysteine), exopolysaccharides. p-cresol, oxoglutaric acid, indole-3-methylacetate, or hydroxyoctadecadienoic acids, or combinations thereof, or isolated bacteria such as Bifidobacteria (e.g., B. infantis, B. longum, B. breve and/or B. bifidum, or a combination thereof), or bacteria genetically modified to overexpress breast milk oligosaccharide metabolizing enzymes, or are modified with galT, ureF, ureC or ureE genes, e.g., from Bifidobacterium longum subsp. infantis. The compositions may include one or more pharmaceutically or neutraceutically acceptable carriers. The compositions can be prepared using any methods known in the art, e.g., added to an existing mixture or formulated as part of a mixture. For example, such compositions can be prepared using acceptable carriers, excipients, or stabilizers (Remington's Pharmaceutical Sciences 16th edition, Osol, A. Ed. (1980); incorporated herein by reference), and in the form of powder or lyophilized formulations or aqueous solutions.

Mixtures of one or more of the agents described herein may be prepared in water suitably mixed with one or more excipients, carriers, or diluents. Dispersions may also be prepared in glycerol, liquid polyethylene glycols, and mixtures thereof and in oils. The forms include aqueous solutions or dispersions and sterile powders for the extemporaneous preparation of sterile solutions or dispersions (e.g., U.S. Pat. No. 5,466,468). In any case, the formulation may be sterile and may be fluid. Formulations may be stable under the conditions of manufacture and storage. The carrier can be a solvent or dispersion medium containing, for example, water, ethanol, polyol (e.g., glycerol, propylene glycol, and liquid polyethylene glycol, and the like), methylcellulose, suitable mixtures thereof, and/or vegetable oils. In many cases, the composition may include isotonic agents, for example, sugars or sodium chloride. In some embodiments, the composition includes methylcellulose. In some embodiments, the composition includes a surfactant (e.g., a poloxamer such as PLURONIC®).

For example, a solution containing a composition described herein may be suitably buffered, if necessary, and the liquid diluent first rendered isotonic with sufficient saline or glucose. In these solutions, sterile aqueous media that can be employed will be known to those of skill in the art in light of the present disclosure. Some variation in dosage may occur depending on the condition of the subject being treated. Moreover, for human administration, preparations may meet sterility, pyrogenicity, general safety, and purity standards as required by FDA Office of Biologics standards.

Administration of the compositions may be continuous or intermittent, depending, for example, upon the recipient's physiological condition, and other factors known to skilled practitioners. The administration of the composition(s) may be essentially continuous over a preselected period of time or may be in a series of spaced doses. Any route of administration may be employed, e.g., oral, or local administration. In one embodiment, the composition is formulated for oral administration. In one embodiment, oral administration is achieved after suspension of a powder composition into a suitable liquid oral vehicle.

The formulations may, where appropriate, be conveniently presented in discrete unit dosage forms and may be prepared by any of the methods well known to the art. Such methods may include the step of bringing into association the active agent with carriers, solid matrices, semi-solid carriers, finely divided solid carriers or combinations thereof, and then, if necessary, introducing or shaping the product into the desired delivery system.

The amount of composition(s) administered to achieve a particular outcome may vary depending on various factors including, but not limited to, the formulation, the condition, patient specific parameters, e.g., height, weight and age, and the like.

Compositions may conveniently be provided in the form of formulations suitable for administration. A suitable administration format may best be determined by a medical practitioner for each patient individually, according to standard procedures. Suitable pharmaceutically acceptable carriers (excipients) and their formulation are described in standard formulations treatises, e.g., Remington's Pharmaceuticals Sciences. By “pharmaceutically acceptable” it is meant a carrier, diluent, excipient, and/or salt that is compatible with the other ingredients of the formulation, and not deleterious to the recipient thereof.

Compositions may be formulated in solution at neutral pH, for example, about pH 6.5 to about pH 8.5, or from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, or from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is useful for buffers containing sodium ions. If desired, solutions of the above compositions can also be prepared to enhance shelf life and stability. Useful compositions can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

Formulations can be prepared by procedures known in the art using well known and readily available ingredients. For example, the composition can be formulated with one or more common excipients, diluents, or carriers, and formed into tablets, capsules, suspensions, powders, and the like. The compositions can also be formulated as elixirs or solutions appropriate for parenteral administration.

The formulations can also take the form of an aqueous or anhydrous solution, e.g., a lyophilized formulation, or dispersion, or alternatively the form of an emulsion or suspension.

The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically or neutraceutically acceptable vehicles and adjuvants which are well known in the prior art. It is possible, for example, to prepare solutions using one or more organic solvent(s) that is/are acceptable from the physiological standpoint.

### Exemplary Compositions and Uses

In one embodiment, a composition having one or more of the disclosed agents is/are provided in powdered or aqueous form in premeasured amounts, e.g., in pouches, for addition to baby formula, breast milk or milk derived from human cells. In one embodiment, a composition having one or more of the disclosed agents is provided in other foods such as snack bars, cookies, gels, or baby food, e.g., solid or semi-solid food. In one embodiment, a baby formula composition having two or more of the disclosed agents is provided in powdered or liquid form, e.g., in individual containers. In one embodiment, the premeasured doses are in a form of pre-dosed, e.g., single use, daily packets, packages, pouches, measured powder supplements, gels, infant formula or other foods.

“Infant” means a human subject ranging in age from birth to not more than one year and includes infants from 0 to 12 months of age.

“Child” means a subject ranging in age from 12 months to about 13 years. In some embodiments, a child is a subject between the ages of 1 and 5 years old.

“Infant formula” or “baby formula” means a composition that satisfies at least a portion of the nutrient requirements of an infant. In the United States, the content of an infant formula is dictated by the federal regulations set forth at 21 C.F.R. Sections 100, 106, and 107. The term “infant formula” also includes starter infant formula and follow-on formula.

The term “starter infant formula” means an infant formula for use during the first four to six months of the life of the infant.

The term “follow-on formula” means an infant formula intended to use by an infant aged from four months or six months to 12 months of age.

In one embodiment, the composition may include a plurality of prebiotics. In certain embodiments, the composition includes prebiotics which can exert additional health benefits, which may include, but are not limited to, selective stimulation of the growth and/or activity of one or a limited number of beneficial gut bacteria, stimulation of the growth and/or activity of ingested probiotic microorganisms, selective reduction in gut pathogens, and favorable influence on gut short chain fatty acid profile. Such prebiotics may be naturally-occurring, synthetic, or developed through the genetic manipulation of organisms and/or plants. Prebiotics include but are not limited to oligosaccharides, polysaccharides, and other prebiotics that contain fructose, xylose, soya, galactose, glucose and mannose. Exemplary prebiotics include but are not limited to lactulose, lactosucrose, raffinose, gluco-oligosaccharide, inulin, fructo-oligosaccharide (FOS), isomalto-oligosaccharide, soybean oligosaccharides, lactosucrose, xylo-oligosaccharide (XOS), chito-oligosaccharide, manno-oligosaccharide, aribino-oligosaccharide, siallyl-oligosaccharide, fuco-oligosaccharide, or gentio-oligosaccharides.

In an embodiment, the total amount of prebiotics present in the composition may be from about 1.0 g/L to about 10.0 g/L of the composition. In one embodiment, the total amount of prebiotics present in the composition may be from about 2.0 g/L and about 8.0 g/L of the composition. In some embodiments, the total amount of prebiotics present in the composition may be from about 0.01 g/100 Kcal to about 1.5 g/100 Kcal. In certain embodiments, the total amount of prebiotics present in the composition may be from about 0.15 g/100 Kcal to about 1.5 g/100 Kcal.

The composition(s) may also comprise a carbohydrate source. Carbohydrate sources can be any used in the art, e.g., lactose, glucose, fructose, corn syrup solids, maltodextrins, sucrose, starch, rice syrup solids, and the like. The amount of carbohydrate in the composition typically can vary from between about 5 g and about 25 g/100 Kcal. In some embodiments, the amount of carbohydrate is between about 6 g and about 22 g/100 Kcal. In other embodiments, the amount of carbohydrate is between about 12 g and about 14 g/100 Kcal. In some embodiments, corn syrup solids are preferred. Moreover, hydrolyzed, partially hydrolyzed, and/or extensively hydrolyzed carbohydrates may be desirable for inclusion in the composition due to their easy digestibility.

Non-limiting examples of carbohydrate materials suitable for use herein include hydrolyzed or intact, naturally or chemically modified, starches sourced from corn, tapioca, rice or potato, in waxy or non-waxy forms. Non-limiting examples of suitable carbohydrates include various hydrolyzed starches characterized as hydrolyzed cornstarch, maltodextrin, maltose, corn syrup, dextrose, corn syrup solids, glucose, and various other glucose polymers and combinations thereof. Non-limiting examples of other suitable carbohydrates include those often referred to as sucrose, lactose, fructose, high fructose corn syrup, indigestible oligosaccharides such as fructooligosaccharides and combinations thereof.

In some embodiments, the composition described herein comprises a fat or lipid source. In certain embodiments, appropriate fat sources include, but are not limited to, animal sources, e.g., milk fat, butter, butter fat, egg yolk lipid; marine sources, such as fish oils, marine oils, single cell oils; vegetable and plant oils, such as corn oil, canola oil, sunflower oil, soybean oil, palm olein oil, coconut oil, high oleic sunflower oil, evening primrose oil, rapeseed oil, olive oil, flaxseed (linseed) oil, cottonseed oil, high oleic safflower oil, palm stearin, palm kernel oil, wheat germ oil; medium chain triglyceride oils and emulsions and esters of fatty acids; and any combinations thereof.

In some embodiment the composition comprises between about 1 g/100 Kcal to about 10 g/100 Kcal of a fat or lipid source. In some embodiments, the composition comprises between about 2 g/100 Kcal to about 7 g/100 Kcal of a fat source. In other embodiments the fat source may be present in an amount from about 2.5 g/100 Kcal to about 6 g/100 Kcal. In still other embodiments, the fat source may be present in the composition in an amount from about 3 g/100 Kcal to about 4 g/100 Kcal.

In some embodiments, the fat or lipid source comprises from about 10% to about 35% palm oil per the total amount of fat or lipid. In some embodiments, the fat or lipid source comprises from about 15% to about 30% palm oil per the total amount of fat or lipid. Yet in other embodiments, the fat or lipid source may comprise from about 18% to about 25% palm oil per the total amount of fat or lipid.

In certain embodiments, the fat or lipid source may be formulated to include from about 2% to about 16% soybean oil based on the total amount of fat or lipid. In some embodiments, the fat or lipid source may be formulated to include from about 4% to about 12% soybean oil based on the total amount of fat or lipid. In some embodiments, the fat or lipid source may be formulated to include from about 6% to about 10% soybean oil based on the total amount of fat or lipid.

In certain embodiments, the fat or lipid source may be formulated to include from about 2% to about 16% coconut oil based on the total amount of fat or lipid. In some embodiments, the fat or lipid source may be formulated to include from about 4% to about 12% coconut oil based on the total amount of fat or lipid. In some embodiments, the fat or lipid source may be formulated to include from about 6% to about 10% coconut oil based on the total amount of fat or lipid.

In certain embodiments, the fat or lipid source may be formulated to include from about 2% to about 16% sunflower oil based on the total amount of fat or lipid. In some embodiments, the fat or lipid source may be formulated to include from about 4% to about 12% sunflower oil based on the total amount of fat or lipid. In some embodiments, the fat or lipid source may be formulated to include from about 6% to about 10% sunflower oil based on the total amount of fat or lipid.

In some embodiments, the oils, e.g., sunflower oil, soybean oil, sunflower oil, palm oil, etc. are meant to cover fortified versions of such oils known in the art. For example, in certain embodiments, the use of sunflower oil may include high oleic sunflower oil. In other examples, the use of such oils may be fortified with certain fatty acids, as known in the art, and may be used in the fat or lipid source disclosed herein.

In some embodiments the composition may also include a source of long chain polyunsaturated fatty acids (LCPUFAs). In one embodiment the amount of LCPUFA in the composition is advantageously at least about 5 mg/100 Kcal, and may vary from about 5 mg/100 Kcal to about 100 mg/100 Kcal, more preferably from about 10 mg/100 Kcal to about 50 mg/100 Kcal. Non-limiting examples of LCPUFAs include, but are not limited to, docosahexanoic acid (DHA) arachidonic acid (ARA), linoleic (18:2 n-6), .gamma.-linolenic (18:3 n-6), dihomo-gamma-linolenic (20:3 n-6) acids in the n-6 pathway, .alpha.-linolenic (18:3 n-3), stearidonic (18:4 n-3), eicosatetraenoic (20:4 n-3), eicosapentaenoic (20:5 n-3), and docosapentaenoic (22:6 n-3).

In some embodiments, the LCPUFA included in the composition may comprise DHA. In one embodiment the amount of DHA in the composition is advantageously at least about 17 mg/100 Kcal, and may vary from about 5 mg/100 Kcal to about 75 mg/100 Kcal, more preferably from about 10 mg/100 Kcal to about 50 mg/100 Kcal.

In another embodiment, if the composition is an infant formula, the composition may be supplemented with both docosahexanoic acid (DHA) and arachidonic acid (ARA). In this embodiment, the weight ratio of ARA:DHA may be between about 1:3 and about 9:1. In a particular embodiment, the ratio of ARA:DHA is from about 1:2 to about 4:1. The DHA and ARA can be in natural form, provided that the remainder of the LCPUFA source does not result in any substantial deleterious effect on the infant. Alternatively, the DHA and ARA can be used in refined form.

The disclosed composition described herein can, in some embodiments, also comprise a source of beta-glucan. Glucans are polysaccharides, specifically polymers of glucose, which are naturally occurring and may be found in cell walls of bacteria, yeast, fungi, and plants. Beta glucans (beta-glucans) are themselves a diverse subset of glucose polymers, which are made up of chains of glucose monomers linked together via beta-type glycosidic bonds to form complex carbohydrates. Beta-1,3-glucans are carbohydrate polymers purified from, for example, yeast, mushroom, bacteria, algae, or cereals. The chemical structure of beta-1,3-glucan depends on the source of the beta-1,3-glucan. Moreover, various physiochemical parameters, such as solubility, primary structure, molecular weight, and branching, play a role in biological activities of beta-1,3-glucans.

Beta-1,3-glucans are naturally occurring polysaccharides, with or without beta-1,6-glucose side chains that are found in the cell walls of a variety of plants, yeasts, fungi and bacteria. Beta-1,3;1,6-glucans are those containing glucose units with (1,3) links having side chains attached at the (1,6) position(s). Beta-1,3;1,6 glucans are a heterogeneous group of glucose polymers that share structural commonalities, including a backbone of straight chain glucose units linked by a beta-1,3 bond with beta-1,6-linked glucose branches extending from this backbone. While this is the basic structure for the presently described class of beta-glucans, some variations may exist. For example, certain yeast beta-glucans have additional regions of beta(1,3) branching extending from the beta(1,6) branches, which add further complexity to their respective structures.

Beta-glucans derived from baker's yeast, Saccharomyces cerevisiae, are made up of chains of D-glucose molecules connected at the 1 and 3 positions, having side chains of glucose attached at the 1 and 6 positions. Yeast-derived beta-glucan is an insoluble, fiber-like, complex sugar having the general structure of a linear chain of glucose units with a beta-1,3 backbone interspersed with beta-1,6 side chains that are generally 6-8 glucose units in length. More specifically, beta-glucan derived from baker's yeast is poly-(1,6)-beta-D-glucopyranosyl-(1,3)-beta-D-glucopyranose.

In some embodiments, the beta-glucan is beta-1,3;1,6-glucan. In some embodiments, the beta-1,3;1,6-glucan is derived from baker's yeast. The composition may comprise whole glucan particle beta-glucan, particulate beta.-glucan, PGG-glucan (poly-1,6-.beta.-D-glucopyranosyl-1,3-.beta.-D-glucopyranose) or any mixture thereof. In some embodiments, the amount of beta-glucan in the composition is between about 3 mg and about 17 mg per 100 Kcal. In another embodiment the amount of beta-glucan is between about 6 mg and about 17 mg per 100 Kcal.

One or more vitamins and/or minerals may also be added in to the composition in amounts sufficient to supply the daily nutritional requirements of a subject. It is to be understood by one of ordinary skill in the art that vitamin and mineral requirements will vary, for example, based on the age of the child. For instance, an infant may have different vitamin and mineral requirements than a child between the ages of one and thirteen years. Thus, the embodiments are not intended to limit the composition to a particular age group but, rather, to provide a range of acceptable vitamin and mineral components.

In embodiments providing a composition for a child, the composition may optionally include, but is not limited to, one or more of the following vitamins or derivations thereof: vitamin B1 (thiamin, thiamin pyrophosphate, TPP, thiamin triphosphate, TTP, thiamin hydrochloride, thiamin mononitrate), vitamin B2 (riboflavin, flavin mononucleotide, FMN, flavin adenine dinucleotide, FAD, lactoflavin, ovoflavin), vitamin B3 (niacin, nicotinic acid, nicotinamide, niacinamide, nicotinamide adenine dinucleotide, NAD, nicotinic acid mononucleotide, NicMN, pyridine-3-carboxylic acid), vitamin B.sub.3-precursor tryptophan, vitamin B6 (pyridoxine, pyridoxal, pyridoxamine, pyridoxine hydrochloride), pantothenic acid (pantothenate, panthenol), folate (folic acid, folacin, pteroylglutamic acid), vitamin B12 (cobalamin, methylcobalamin, deoxyadenosylcobalamin, cyanocobalamin, hydroxycobalamin, adenosylcobalamin), biotin, vitamin C (ascorbic acid), vitamin A (retinol, retinyl acetate, retinyl palmitate, retinyl esters with other long-chain fatty acids, retinal, retinoic acid, retinol esters), vitamin D (calciferol, cholecalciferol, vitamin D.sub.3, 1,25,-dihydroxyvitamin D), vitamin E (alpha-tocopherol, alpha-tocopherol acetate, .alpha.-tocopherol succinate, .alpha.-tocopherol nicotinate, .alpha.-tocopherol), vitamin K (vitamin K1, phylloquinone, naphthoquinone, vitamin K2, menaquinone-7, vitamin K3, menaquinone-4, menadione, menaquinone-8, menaquinone-8H, menaquinone-9, menaquinone-9H, menaquinone-10, menaquinone-11, menaquinone-12, menaquinone-13), choline, inositol, beta-carotene and any combinations thereof.

In embodiments providing a children's product, such as a growing-up milk, the composition may optionally include, but is not limited to, one or more of the following minerals or derivations thereof: boron, calcium, calcium acetate, calcium gluconate, calcium chloride, calcium lactate, calcium phosphate, calcium sulfate, chloride, chromium, chromium chloride, chromium picolonate, copper, copper sulfate, copper gluconate, cupric sulfate, fluoride, iron, carbonyl iron, ferric iron, ferrous fumarate, ferric orthophosphate, iron trituration, polysaccharide iron, iodide, iodine, magnesium, magnesium carbonate, magnesium hydroxide, magnesium oxide, magnesium stearate, magnesium sulfate, manganese, molybdenum, phosphorus, potassium, potassium phosphate, potassium iodide, potassium chloride, potassium acetate, selenium, sulfur, sodium, docusate sodium, sodium chloride, sodium selenate, sodium molybdate, zinc, zinc oxide, zinc sulfate and mixtures thereof. Non-limiting exemplary derivatives of mineral compounds include salts, alkaline salts, esters and chelates of any mineral compound.

The minerals can be added in the form of salts such as calcium phosphate, calcium glycerol phosphate, sodium citrate, potassium chloride, potassium phosphate, magnesium phosphate, ferrous sulfate, zinc sulfate, cupric sulfate, manganese sulfate, and sodium selenite. Additional vitamins and minerals can be added as known within the art.

The compositions may optionally include one or more of the following flavoring agents, including, but not limited to, flavored extracts, volatile oils, cocoa or chocolate flavorings, peanut butter flavoring, cookie crumbs, vanilla or any commercially available flavoring. Examples of useful flavorings include, but are not limited to, pure anise extract, imitation banana extract, imitation cherry extract, chocolate extract, pure lemon extract, pure orange extract, pure peppermint extract, honey, imitation pineapple extract, imitation rum extract, imitation strawberry extract, or vanilla extract; or volatile oils, such as balm oil, bay oil, bergamot oil, cedarwood oil, cherry oil, cinnamon oil, clove oil, or peppermint oil; peanut butter, chocolate flavoring, vanilla cookie crumb, butterscotch, toffee, and mixtures thereof. The amounts of flavoring agent can vary greatly depending upon the flavoring agent used. The type and amount of flavoring agent can be selected as is known in the art.

The compositions may optionally include one or more emulsifiers that may be added for stability of the final product. Examples of suitable emulsifiers include, but are not limited to, lecithin (e.g., from egg or soy), alpha lactalbumin and/or mono- and di-glycerides, and mixtures thereof. Other emulsifiers are readily apparent to the skilled artisan and selection of suitable emulsifier(s) will depend, in part, upon the formulation and final product. Indeed, the incorporation of a blend of intact protein, protein hydrolysates, and amino acids into a composition, such as an infant formula, may require the presence of at least on emulsifier to ensure that the blend of intact protein, hydrolysates, and amino acids do not separate from the fat or proteins contained within the infant formula during shelf-storage or preparation.

In some embodiments, the composition may be formulated to include from about 0.5 wt % to about 1 wt % of emulsifier based on the total dry weight of the composition. In other embodiments, the composition may be formulated to include from about 0.7 wt % to about 1 wt % of emulsifier based on the total dry weight of the composition.

In some embodiments where the composition is a ready-to-use liquid composition, the composition may be formulated to include from about 200 mg/L to about 600 mg/L of emulsifier. Still, in certain embodiments, the composition may include from about 300 mg/L to about 500 mg/L of emulsifier. In other embodiments, the composition may include from about 400 mg/L to about 500 mg/L of emulsifier.

The compositions may optionally include one or more preservatives that may also be added to extend product shelf life. Suitable preservatives include, but are not limited to, potassium sorbate, sodium sorbate, potassium benzoate, sodium benzoate, potassium citrate, calcium disodium EDTA, and mixtures thereof. The incorporation of a preservative in the composition including a blend of intact protein, protein hydrolysates, and/or amino acids ensures that the composition has a suitable shelf-life such that, once reconstituted for administration, the composition delivers nutrients that are bioavailable and/or provide health and nutrition benefits for the target subject.

In some embodiments the composition may be formulated to include from about 0.1 wt % to about 1.0 wt % of a preservative based on the total dry weight of the composition. In other embodiments, the composition may be formulated to include from about 0.4 wt % to about 0.7 wt % of a preservative based on the total dry weight of the composition.

In some embodiments where the composition is a ready-to-use liquid composition, the composition may be formulated to include from about 0.5 g/L to about 5 g/L of preservative. Still, in certain embodiments, the composition may include from about 1 g/L to about 3 g/L of preservative.

The composition may optionally include one or more stabilizers. Suitable stabilizers for use in practicing the composition of the present disclosure include, but are not limited to, gum arabic, gum ghatti, gum karaya, gum tragacanth, agar, furcellaran, guar gum, gellan gum, locust bean gum, pectin, low methoxyl pectin, gelatin, microcrystalline cellulose, CMC (sodium carboxymethylcellulose), methylcellulose hydroxypropyl methyl cellulose, hydroxypropyl cellulose, DATEM (diacetyl tartaric acid esters of mono- and diglycerides), dextran, carrageenans, and mixtures thereof. Indeed, incorporating a suitable stabilizer in the composition including intact protein, protein hydrolysates, and/or amino acids ensures that the composition has a suitable shelf-life such that, once reconstituted for administration, the composition delivers nutrients that are bioavailable and/or provide health and nutrition benefits for the target subject.

In some embodiments where the composition is a ready-to-use liquid composition, the composition may be formulated to include from about 50 mg/L to about 150 mg/L of stabilizer. Still, in certain embodiments, the composition may include from about 80 mg/L to about 120 mg/L of stabilizer.

In an embodiment, the children's composition may contain between about 10 and about 50% of the maximum dietary recommendation for any given country, or between about 10 and about 50% of the average dietary recommendation for a group of countries, per serving of vitamins A, C, and E, zinc, iron, iodine, selenium, and choline. In another embodiment, the children's composition may supply about 10-30% of the maximum dietary recommendation for any given country, or about 10-30% of the average dietary recommendation for a group of countries, per serving of B-vitamins. In yet another embodiment, the levels of vitamin D, calcium, magnesium, phosphorus, and potassium in the children's nutritional product may correspond with the average levels found in milk. In other embodiments, other nutrients in the children's composition may be present at about 20% of the maximum dietary recommendation for any given country, or about 20% of the average dietary recommendation for a group of countries, per serving.

In some embodiments the composition is an infant formula. Infant formulas are fortified compositions for an infant. The content of an infant formula is dictated by federal regulations, which define macronutrient, vitamin, mineral, and other ingredient levels in an effort to simulate the nutritional and other properties of human breast milk. Infant formulas are designed to support overall health and development in a pediatric human subject, such as an infant or a child.

In some embodiments, the composition of the present disclosure is a growing-up milk. Growing-up milks are fortified milk-based beverages intended for children over 1 year of age (typically from 1-3 years of age, from 4-6 years of age or from 1-6 years of age). Growing-up milks are designed with the intent to serve as a complement to a diverse diet to provide additional insurance that a child achieves continual, daily intake of all essential vitamins and minerals, macronutrients plus additional functional dietary components, such as non-essential nutrients that have purported health-promoting properties.

The exact composition of a growing-up milk or other composition according to the present disclosure can vary from market-to-market, depending on local regulations and dietary intake information of the population of interest. In some embodiments, compositions according to the disclosure consist of a milk protein source, such as whole or skim milk, plus added sugar and sweeteners to achieve desired sensory properties, and added vitamins and minerals. The fat composition includes an enriched lipid fraction derived from milk. Total protein can be targeted to match that of human milk, cow milk or a lower value. Total carbohydrate is usually targeted to provide as little added sugar, such as sucrose or fructose, as possible to achieve an acceptable taste. Typically, Vitamin A, calcium and Vitamin D are added at levels to match the nutrient contribution of regional cow milk. Otherwise, in some embodiments, vitamins and minerals can be added at levels that provide approximately 20% of the dietary reference intake (DRI) or 20% of the Daily Value (DV) per serving. Moreover, nutrient values can vary between markets depending on the identified nutritional needs of the intended population, raw material contributions and regional regulations.

The disclosed composition(s) may be provided in any form known in the art, such as a powder, a gel, a suspension, a paste, a solid, a liquid, a liquid concentrate, a reconstitutable powdered milk substitute or a ready-to-use product. The composition may, in certain embodiments, comprise a nutritional supplement, children's nutritional product, infant formula, human milk fortifier, growing-up milk or any other composition designed for an infant or a pediatric subject. Compositions of the present disclosure include, for example, orally-ingestible, health-promoting substances including, for example, foods, beverages, tablets, capsules and powders. Moreover, the composition of the present disclosure may be standardized to a specific caloric content, it may be provided as a ready-to-use product, or it may be provided in a concentrated form.

The compositions may be provided in a suitable container system. For example, non-limiting examples of suitable container systems include plastic containers, metal containers, foil pouches, plastic pouches, multi-layered pouches, and combinations thereof. In certain embodiments, the composition may be a powdered composition that is contained within a plastic container. In certain other embodiments, the composition may be contained within a plastic pouch located inside a plastic container.

### EXEMPLARY EMBODIMENTS

In one embodiment, a method to detect immune health status in a human infant or child is provided. The method includes providing a stool sample from a human infant or child; and determining in the sample i) the relative abundance of bacteria including two or more of Bacteroides, Bifidobacterium, or Blautia, ii) the relative abundance of bacteria including two or more of Bifidobacterium bifidum, Bifidobacterium breve, Bifidobacterium longum, or Bifidobacterium pseudocatenulatum, or iii) the relative abundance or expression of one, two or more of Blon_0915, Blon_2177, Blon_0625, Blon_0244, Blon_0248; Blon_0426, ureF, Blon_0113, ureC Blon_0111, ureE Blon_0112, BLIJ_0113, Blon_0642, Blon_2336, Blon_2344, or Blon_0650 or one, two or more of H1 (Blon_2331-2361), H2 (Blon_0243-Blon_0248), H3 (Blon_0247, Blon_0244-Blon_0248), H4 (Blon_0625; Blon_0641-Blon_0651), or Urease (Blon_0104-Blon_0115). In one embodiment, the child is less than about 5 years old. In one embodiment, a relative abundance of Bacteroides of >10%, of Bifidobacterium of <60% or of Blautia of >10% is indicative of an infant or child at increased risk of allergies or other diseases or a relative abundance of Bacteroides of >8%, of Bifidobacterium of <65% or of Blautia of >2% is indicative of an infant or child at increased risk of allergies or other diseases. In one embodiment, a relative abundance of Bacteroides of >10%, of Bifidobacterium of <60% and of Blautia of >10% is indicative of an infant or child at increased risk of allergies or other diseases or a relative abundance of Bacteroides of >8%, of Bifidobacterium of <65% and of Blautia of >2% is indicative of an infant or child at increased risk of allergies or other diseases. In one embodiment, a relative abundance of Bacteroides of <10%, of Bifidobacterium of >60% or of Blautia of <10% is indicative of an infant or child at decreased risk of allergies or other diseases or Bacteroides of <10%, of Bifidobacterium of >65% or of Blautia of <2% is indicative of an infant or child at decreased risk of allergies or other diseases. In one embodiment, a relative abundance of Bacteroides of <10%, of Bifidobacterium of >60% and of Blautia of <10% is indicative of an infant or child at decreased risk of allergies or other diseases or Bacteroides of <10%, of Bifidobacterium of >65% or of Blautia of <2% is indicative of an infant or child at decreased risk of allergies or other diseases. In one embodiment, a relative abundance of Bifidobacterium bifidum of 5% to 10%, Bifidobacterium breve of 2% to 25%, Bifidobacterium longum of 25% or greater, or of Bifidobacterium pseudocatenulatum of less than 2% is indicative of immune health in the infant or child. In one embodiment, a relative abundance of Bifidobacterium bifidum of 10% or less, Bifidobacterium breve of 25% or less, Bifidobacterium longum of 25% or greater, or of Bifidobacterium pseudocatenulatum of less than 2% is indicative of immune health in the infant or child or of Bifidobacterium breve of 15% or less, Bifidobacterium longum of 65% or greater, or of Bifidobacterium pseudocatenulatum of less than 3% is indicative of immune health in the infant or child. In one embodiment, a relative abundance of Bifidobacterium bifidum of 10% or less, Bifidobacterium breve of 25% or less, Bifidobacterium longum of 25% or greater, and of Bifidobacterium pseudocatenulatum of less than 2% is indicative of immune health in the infant or child or of Bifidobacterium breve of 15% or less, Bifidobacterium longum of 65% or greater, and of Bifidobacterium pseudocatenulatum of less than 3% is indicative of immune health in the infant or child. In one embodiment, a relative abundance of Bifidobacterium bifidum of 5% or greater, Bifidobacterium breve of 20% or less, Bifidobacterium longum of 50% or greater, or of Bifidobacterium pseudocatenulatum of less than 2% is indicative of immune health in the infant or child. In one embodiment, a relative abundance of Bifidobacterium bifidum of 5% or greater, Bifidobacterium breve of 20% or less, Bifidobacterium longum of 50% or greater, and of Bifidobacterium pseudocatenulatum of less than 2% is indicative of immune health in the infant or child. In one embodiment, a relative abundance of Bifidobacterium bifidum of less than 5%, Bifidobacterium breve of greater than 20%, Bifidobacterium longum of less than 50%, or of Bifidobacterium pseudocatenulatum of greater than 2% is indicative of impaired immune health in the infant or child or of Bifidobacterium breve of greater than 15%, Bifidobacterium longum of less than 30%, or of Bifidobacterium pseudocatenulatum of greater than 3% is indicative of impaired immune health in the infant or child. In one embodiment, an increase in the relative abundance of expression of two or more of Blon_0915, Blon_2171, Blon_2173, Blon_2334, galT Blon_2172, Blon_0244, Blon_0248; Blon_0426, ureF Blon_0113, ureC Blon_0111, ureE Blon_0112 BLIJ_0113, Blon_0642, Blon_2336, Blon_2344, or Blon_0650, or of two or more of H1 (Blon_2331-2361), H2 (Blon_0243-Blon_0248), H3 (Blon_0247, Blon_0244-Blon_0248), H4 (Blon_0625; Blon_0641-Blon_0651), and Urease (Blon_0104-Blon_0115) is indicative of immune health in the infant or child. In one embodiment, the sample is from a newborn. In one embodiment, the sample is from a newborn up to a 3 month old infant. In one embodiment, the sample is from a 3 month old up to a 9 month old infant. In one embodiment, the sample is from an infant or child treated with a drug. In one embodiment, the drug is an antibiotic. In one embodiment, the infant or child has necrotizing enterocolitis. In one embodiment, the method further comprising administering to the mother of the infant or child, or a pregnant mother, a composition optionally comprising one or more prebiotics or one or more probiotics. In one embodiment, the prebiotic or probiotic comprises one or more bacteria, one or more antibodies, or one or more molecules that enhance the relative abundance of Bifidobacterium longum. In one embodiment, the relative abundance of Bifidobacterium longum infantis is enhanced. In one embodiment, the relative abundance of Bifidobacterium longum infantis is greater than 60%, 70%, 80% or 90% after taking the composition. In one embodiment, the sample is analyzed using a nucleic acid amplification reaction. In one embodiment, the sample is analyzed using genome sequencing.

Further provided is a method to identify a human infant or child at higher risk of developing allergies as an adolescent or adult, comprising: providing a stool sample from a human infant or child; and determining in the sample i) the relative abundance of bacteria including two or more of Bacteroides, Bifidobacterium, or Blautia, ii) the relative abundance of bacteria including two or more of Bifidobacterium bifidum, Bifidobacterium breve, Bifidobacterium longum, or Bifidobacterium pseudocatenulatum, or iii) the relative abundance or expression of two or more of Blon_0915, Blon_2177, Blon_0625, Blon_0244, Blon_0248; Blon_0426, ureF Blon_0113, ureC Blon_0111, ureE Blon_0112 BLIJ_0113, Blon_0642, Blon_2336, Blon_2344, or Blon_0650.

In one embodiment, a kit is provided comprising a plurality of probes or primers to determine i) the relative abundance of bacteria including two or more of Bacteroides, Bifidobacterium, or Blautia in a physiological sample, ii) the relative abundance of bacteria including two or more of Bifidobacterium bifidum, Bifidobacterium breve, Bifidobacterium longum, or Bifidobacterium pseudocatenulatum in a physiological sample, or iii) the relative abundance or expression of two or more of Blon_0915, Blon_2177, Blon_0625, Blon_0244, Blon_0248; Blon_0426, ureF Blon_0113, ureC Blon_0111, ureE Blon_0112 BLIJ_0113, Blon_0642, Blon_2336, Blon_2344, or Blon_0650 in a physiological sample.

Also provided is a method to detect immune health status in a human infant or child, comprising: providing a stool sample from a human infant or child; and determining in the sample i) the relative abundance of Bifidobacterium, or Blautia, ii) the relative abundance of bacteria including one or more of Bifidobacterium bifidum, Bifidobacterium breve, Bifidobacterium longum, or Bifidobacterium pseudocatenulatum, or iii) the relative abundance or expression of one or more of Blon_0915, Blon_2177, Blon_0625, Blon_0244, Blon_0248; Blon_0426, ureF, Blon_0113, ureC Blon_0111, ureE Blon_0112, BLIJ_0113, Blon_0642, Blon_2336, Blon_2344, or Blon_0650. In one embodiment, the relative abundance of Bifidobacterium is >60%. In one embodiment, the relative abundance of Bifidobacterium bifidum, Bifidobacterium breve, Bifidobacterium longum, or Bifidobacterium pseudocatenulatum is >60%. In one embodiment, the relative abundance of Bifidobacterium Bifidobacterium longum is >60%.

The invention will be described by the following non-limiting examples.

### Example 1

There are circumstances that might prevent a mother from breastfeeding or an infant or child may require the use of antibiotics, leading to a reduced prevalence of key bacterial species in an infant or child's gut. In the first months of birth, the loss of Bifidobacterium species, particularly Bifidobacterium longum infantis, or gain of other bacteria during this window of opportunity, may significantly alter the ‘natural’ progression of the microbial community that may lead to a variety of negative consequences for host health including a predisposition to autoimmune, metabolic, and neurobehavioral diseases (such as IBD, allergies, childhood obesity, ADHD, and autism). A recent report profiling children's gut microbiomes in the United States clearly show an overall low abundance of Bifidobacterium genus in infants 0-3 months of age. There is an unmet need to provide alternatives to infant formula for better nutrition that promote health and well-being.

It is highly likely that human breast milk HMOs are not the sole promoters of a healthy gut microbiome. The Wisconsin Infant Study Cohort (WISC) birth cohort (U19 AI104317, MPI Gern/Seroogy) consists of three distinct study arms (animal farming study group, rural non-farming study group, and TA study group) aimed at characterizing the impact of early life farming exposures on immune development, respiratory health, and allergic diseases Stool sample collected from study infants at 2 months of age underwent shotgun metagenomic sequencing. As disclosed herein, there is an increased abundance (80%) of several Bifidobacteria species in the TA infant study group compared to the non-TA infants (50%). Specifically, the predominant strain comprising ˜75% of the bacterial composition of TA infant's gut microbial community consists of one species: Bifidobacterium longum infantis, whereas the non-TA infants comprise ˜30% of Bifidobacterium longum subsp. longum. Importantly, this is controlled for breast feeding. The difference in abundance amongst breastfed infants is and strongly suggests that differences in breast milk components are impacting the predominance of Bifidobacterium longum infantis.

### Materials and Methods

Recruitment. Study participants for the WISC Farm and Nonfarm study arms were recruited from families receiving prenatal care at the Marshfield Clinic (various locations across Wisconsin), and for the WFS arm, the LaFarge Birthing Center (LaFarge, WI).

Stool sample selection. Stool was collected from study participants at approximately 2 months of age. The allowed collection window spanned 1.5 to 6 months of age, with most samples falling close to the two month date. DNA from stool samples had been previously extracted and frozen. To select samples for shotgun metagenomics sequencing, we included all children in the WFS study arm for whom at least 100 ng DNA was available (n=27). To select Farm and Nonfarm samples with matching attributes, samples from infants with vaginal deliveries, who were exclusively breastfed at the time of sample collection, and who enrolled in the study close to the same time as the TA participants, were analyzed. A total of 46 Farm and 43 Nonfarm samples were analyzed.

### Metagenomics and Sample Preparation

Sample preparation for sequencing. DNA was extracted from stool using a modified cetyltrimethylammonium bromide (CTAB)-buffer-based protocol (DeAngelis et al. 2009), as described previously by Fujimura et al (Fujimura et al. 2016). Metagenomic shotgun library preparation and sequencing were performed at the DNA Sequencing Facility at the University of Wisconsin-Madison on the Illumina® NovaSeq 6000 platform using a paired-end sequencing approach with a targeted read length of 150 bp.

### Primary Processing of Metagenomics and MS Data

Basic processing of metagenomics sequencing data. Initial processing, taxonomic classification, and functional profiling of metagenomics samples was performed using bioBakery3 utilities (Beghini et al. 2021). KneadData was applied for automated quality control, which included quality trimming and removal of reads that map to the human genome (hg38). MetaPhlan v3 (Segata et al. 2012; Beghini et al. 2021) was applied for taxonomic classification and computation of relative abundance matrices.

### Downstream Analysis

Bifidobacterium longum gene family detection. To inspect Bifidobacterium longum gene presence in the samples, PanPhlan (Beghini et al. 2021) was used to evaluate the presence/absence of UniRef90 gene families identified in a Bifidobacterium longum pangenome that was computed by uniting several reference genomes. The pangenome was provided with the PanPhlan software.

Identifying differentially abundant microbes between TA and non-TA. We applied LEfSe (Segata et al. 2011), which uses Kruskal-Wallis sum-rank tests to evaluate whether a taxa is significantly different between study groups, followed by estimating the effect sizes of those differences using Linear Discriminant Analysis (LDA) with bootstrap resampling. Centered log ratio (CLR) transformation, per sample, was used to the relative abundance matrix prior to running LEfSE. Microbes were accepted at p<0.05 with LDA score of at least 2.

Functional analysis. HUMANn (Franzosa et al. 2018; Beghini et al. 2021) was used to estimate copies per million for UniRef90 gene families and MetaCyc™ Pathways. The first output of this approach is an estimated Copies per Million (CPM) for UniRef90 gene families within each sample. Each UniRef90 gene family is a cluster of genes from one or more taxa that were assigned based on a 90% sequence identity. Under each UniRef90 gene family, HUMANn® also provides estimates of the CPMs for the taxa-specific genes within the family. MetaCyc™ pathway CPMs are estimated by aggregating the CPMs for gene families assigned to each MetaCyc™ pathway. Taxa-specific estimates are also provided for each pathway when possible.

The CPM was inspected and an infantis marker gene (Blon_0915) and genes involved in HMO metabolism (LoCascio et al. 2010) were identified. Of the 56 genes identified by LoCascio et al, 15 were found in the HUMANn® gene families results file.

Identifying pathways associated with TA vs. Non-TA. Linear modeling, implemented in Maaslin2 (Mallick et al. 2021), was used to identify MetaCyc™ pathways that are differentially abundant at the community level between the TA and non-TA cohorts. The analysis was of infants who were exclusively breastfeeding at the time of sample collection. The statistical test was performed on the community-level total for each pathway, and accepted as significant those with p-value <0.01 after adjustment using the Benjamini-Hochberg procedure. For significant pathways, the taxa-specific distribution of the CPMs was visibly inspected to interpret the result. A stricter adjusted p-value threshold was used for this analysis compared to others (in other words, a threshold lower than 0.05) in order to prioritize a reasonable number of results for manual investigation.

Machine learning. The tidymodels (Kuhn and Wickham 2020) R libraries were used to build classifiers to discriminate TA from non-TA samples using the estimated microbial abundances. To reduce the potential of learning dietary differences (formula vs. breastmilk) instead of farm exposure differences, the analysis was conducted on exclusively breastfeeding children.

Data preparation. W Features with near zero variance (defined as having less than 5% unique values, or a ratio of most-common value to second-most common level greater than 95/5) were removed. Relative abundances were converted using a modified mean-centered log ratio (computing the means using non-zero values) and ran the analysis with two versions of the features: features at all levels of the phylogeny (all_levels) as well as only species-level features (species). Results are shown for species-level predictions.

Modeling algorithms. For each of the following models, a tuning parameter grid of 20 parameters was generated in the default range for each parameter (defined in tidymodels model specifications).
random forest (randomForest and ranger)
elastic net (glmnet)
linear support vector machine (kernlab)
boosted gradient trees (xgboost)
k-nearest neighbor
   For ranger and randomForest, we also ran with a set of default parameters: 1000 trees, mtry=sqrt(number of features) (number of random feature choices to consider at each split).

Model selection and evaluation. For each model, 10 repeats of nested cross-validation were run with 10 outer training/testing folds. For each outer fold, a five-fold cross-validation was used on the training set to estimate the performance of each parameter setting. The parameters selected were based on the area under the precision-recall curve (PR-AUC), and trained a single model for that training fold to make predictions on its paired testing fold. The predictions from all 10 folds were concatenated before computing evaluation metrics: PR-AUC and area under the receiver operating characteristic curve (ROC-AUC).

Variable importance. The 10 repeats of ten-fold cross-validation ultimately resulted in 100 trained models per algorithm. The top performing methods for variable interpretation were selected: glmnet and ranger, default parameters (which tied with randomForest, default parameters). The variable importance for each model was estimated and each feature summarized by the median importance across all 100 models (where an importance of 0 means that the feature was not used). For glmnet, the variable importance is the absolute value of the standardized coefficient. For ranger, the variable importance is the Gini Impurity, or the feature's mean improvement in the split criterion (decrease in node impurity) across the forest.

Miscellaneous libraries for computational analysis. In the course of this analysis, data structures and functions provided in the R libraries phyloseq (McMurdie and Holmes 2013), microbiome, ggplot2, tidyverse, were used.

### Results

Shotgun metagenomics sequencing was used to profile the two-month-old gut microbiome of 116 infants, comprising 27 infants from TA families (referred to as WFS cohort from this point on), 46 from farming families (Farm cohort), and 43 from non-farming families (Nonfarm cohort). Compared to Farm and Nonfarm, the WFS families had a larger number of children living in the home, lower maternal age, and a higher rate of male to female infants in the study (Table 1). Nearly all WFS mothers consumed unprocessed farm milk during pregnancy, while this was rare among the others.

TABLE 1
Study participant demographics and characteristics.

| value | Amish | Farm | Nonfarm |
|---|---|---|---|
| batch | | | |
| stool_2021_07_08 | 0.44 (12/27) | 0 | 0.09 (4/43) |
| stool_bmilk_2020_07_15 | 0.56 (15/27) | 0.43 (20/46) | 0.70 (30/43) |
| stool_bmilk_ | 0 | 0.50 (23/46) | 0.14 (6/43) |
| hsdust_2021_03_17 | | | |
| stool02_metagenomics_ | 0 | 0.07 (3/46) | 0.07 (3/43) |
| 2019_09_04 | | | |
| birthmonthcat | | | |
| December-February | 0.15 (4/27) | 0.22 (10/46) | 0.26 (11/43) |
| June-August | 0.22 (6/27) | 0.28 (13/46) | 0.21 (9/43) |
| March-May | 0.41 (11/27) | 0.24 (11/46) | 0.28 (12/43) |
| September-November | 0.22 (6/27) | 0.26 (12/46) | 0.26 (11/43) |
| delivery_mode | | | |
| vaginal | 1.00 (27/27) | 0.85 (39/46) | 0.93 (40/43) |
| c-section | 0 | 0.15 (7/46) | 0.07 (3/43) |
| momagecat | | | |
| >=40 | 0.08 (2/26) | 0.02 (1/46) | 0 |
| 18-24 | 0.27 (7/26) | 0.09 (4/46) | 0.05 (2/43) |
| 24-30 | 0.38 (10/26) | 0.39 (18/46) | 0.37 (16/43) |
| 30-34 | 0.12 (3/26) | 0.28 (13/46) | 0.42 (18/43) |
| 34-39 | 0.15 (4/26) | 0.22 (10/46) | 0.16 (7/43) |
| season_cat | | | |
| December-February | 0 | 0.26 (12/46) | 0.19 (8/43) |
| June-August | 0 | 0.33 (15/46) | 0.16 (7/43) |
| March-May | 0 | 0.17 (8/46) | 0.37 (16/43) |
| September-November | 0 | 0.24 (11/46) | 0.28 (12/43) |
| sex | | | |
| Female | 0.33 (9/27) | 0.41 (19/46) | 0.56 (24/43) |
| Male | 0.67 (18/27) | 0.59 (27/46) | 0.44 (19/43) |
| Total | | | |
| Count | 27 | 46 | 43 |

The Infant Gut Microbiome is Associated with Diet and Farming Exposures

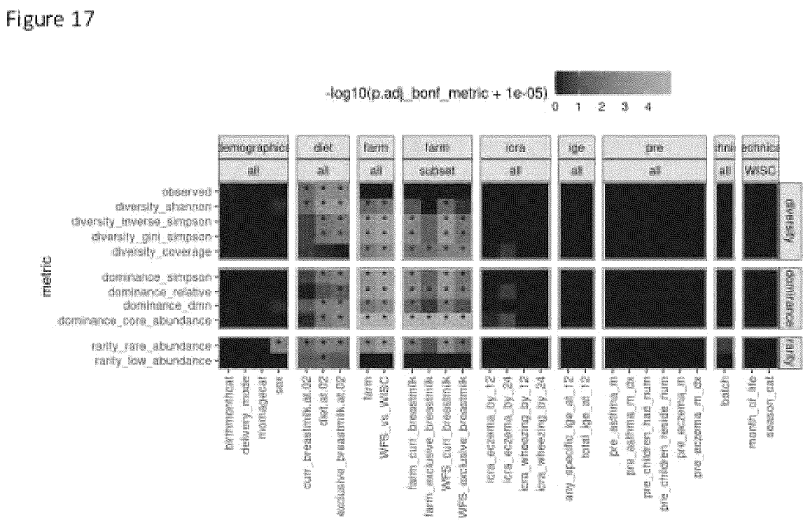

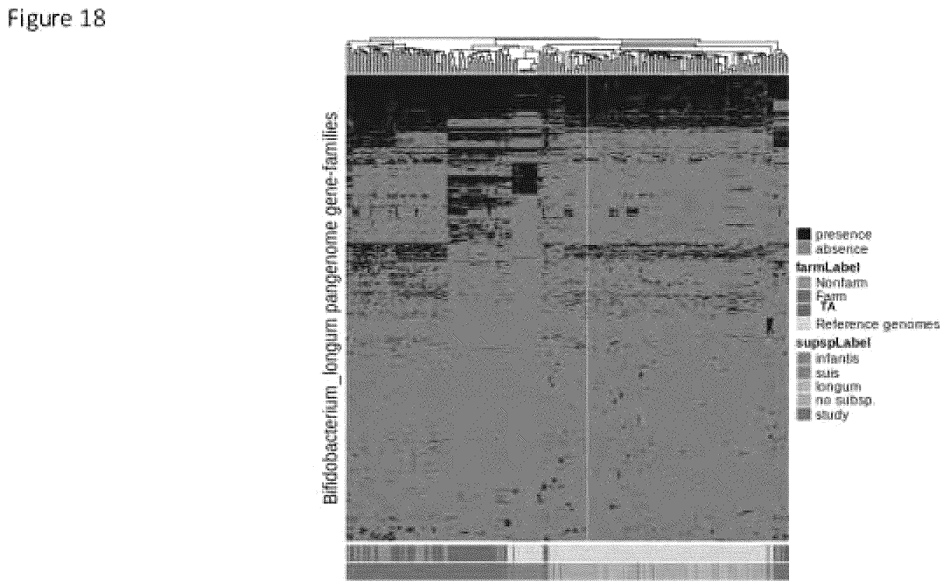

Alpha diversity metrics can provide a high-level description of the richness and distributional qualities of metagenomics samples. Various alpha diversity metrics were tested for association with sample variables including technical variables, demographics, family history of asthma and atopic dermatitis (eczema), and infant eczema, wheezing, and sensitization outcomes at one and two years (FIG. 17). Diversity metrics were associated with infant diet and farming groups, but no others reached significance. Exclusive breastfeeding was associated with low species richness compared to children who were fed formula along with breastmilk or exclusively. Farm group was associated with dominance of core taxa. The sample-sample similarity structure also separated samples by study group and diet) based on PERMANOVA tests.

Visualization of highly prevalent genera and species (at least 1% relative abundance in at least 10% of study samples) quickly provided a simple explanation for the alpha diversity metrics that were associated with farm and diet: exclusively breastfeeding participants were characterized by high relative abundance of Bifidobacterium species, with Bifidobacterium longum particularly high in TA participants.

Next, the distribution of Bifidobacterium species among the exclusively breastfeeding participants was examined. Strikingly, the microbiota of TA participants were dominated by Bifidobacterium longum and to a lesser extent bifidum, while the non-TA participants displayed a more varied profile with high abundance of longum, bifidum, breve, and pseudocatenulatum.

Bifidobacterium longum Subsp. Infantis Genes are Found in WFS Infants

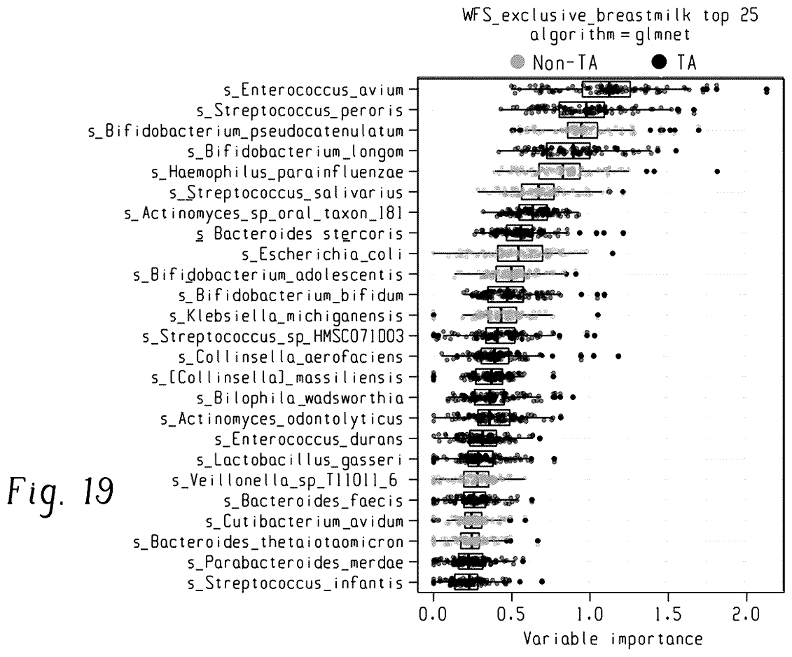

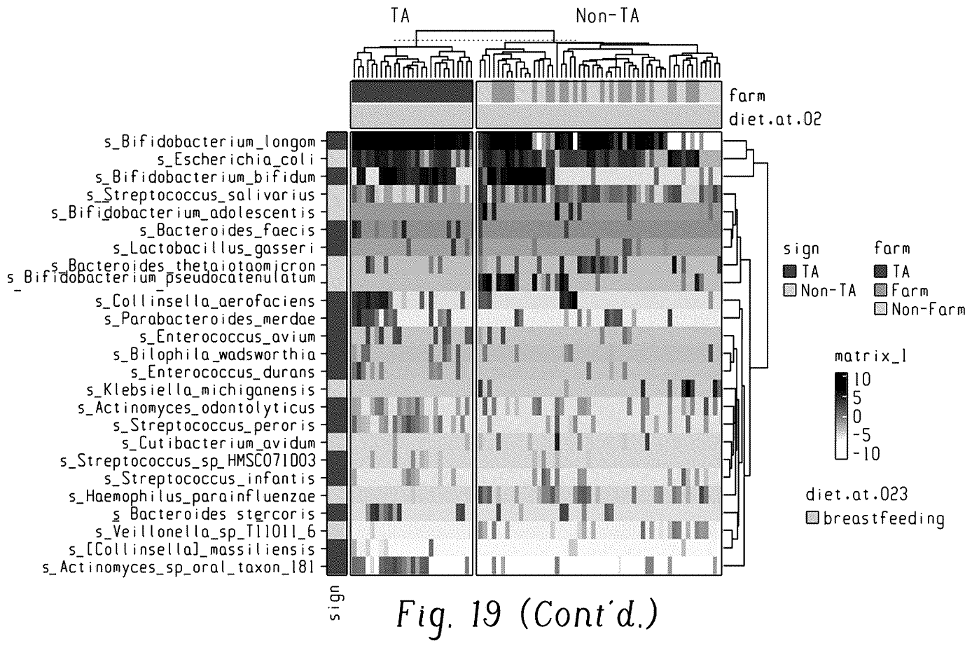

A gene-level assessment of the genetic diversity of Bifidobacterium longum in the study samples (FIG. 19) was conducted. PanPhlan (Scholz et al., 2016) was used to evaluate the presence or absence of B. longum genes (that is, UniRef90 clusters identified in B. longum reference genomes). The profiles of TA participants clustered together distinctly from the non-TA, and also clustered with the reference genomes that were labeled as Bifidobacterium longum subsp. infantis.

TABLE 2

| HMO | UniRef90 | | |
|---|---|---|---|
| Cluster | Gene Family | Blon_gene | Protein_name |
| H1 | UniRef90_B7GNN8 | Blon_2336 | Alpha-1,3/4-fucosidase, |
| | | | putative |
| H1 | UniRef90_B7GNP6 | Blon_2344 | Extracellular solute- |
| | | | binding protein, family 1 |
| H1 | UniRef90_Q8G5N0 | Blon_2334 | Beta-galactosidase |
| | | | (EC 3.2.1.23) (Lactase) |
| H2 | UniRef90_B7GN40 | Blon_0248 | Alpha-L-fucosidase |
| | | | (EC 3.2.1.51) |
| H2 | UniRef90_B7GTT2 | Blon_0244 | Signal transduction |
| | | | histidine kinase-like |
| | | | protein |
| H3 | UniRef90_B7GN40 | Blon_0426 | Alpha-L-fucosidase |
| | | | (EC 3.2.1.51) |
| H4 | UniRef90_ | Blon_0650 | ABC transporter related |
| | A0A087BR20 | | |
| H4 | UniRef90_B7GPL9 | Blon_0642 | GntR domain protein |
| H4 | UniRef90_E7CY69 | Blon_0625 | Beta-glucosidase |
| | | | (EC 3.2.1.21) |
| H5 | UniRef90_B3DQG9 | Blon_2177 | Extracellular solute- |
| | | | binding protein, family 1 |
| H5 | UniRef90_E8MF10 | Blon_2171 | UDP-glucose 4-epimerase |
| | | | (EC 5.1.3.2) |
| H5 | UniRef90_E8MF11 | galT | Galactose-1-phosphate |
| | | Blon_2172 | uridylyltransferase (Gal-1-P |
| | | | uridylyltransferase) |
| | | | (EC 2.7.7.12) (UDP- |
| | | | glucose--hexose-1-phosphate |
| | | | uridylyltransferase) |
| H5 | UniRef90_E8MF12 | Blon_2173 | Aminoglycoside |
| | | | phosphotransferase |
| Urease | UniRef90_B7GT17 | ureC | Urease subunit alpha |
| | | Blon_0111 | (EC 3.5.1.5) (Urea |
| | | | amidohydrolase |
| | | | subunit alpha) |
| Urease | UniRef90_B7GT18 | ureE | Urease accessory |
| | | Blon_0112 | protein UreE |
| | | BLIJ_0113 | |
| Urease | UniRef90_B7GT19 | ureF | Urease accessory |
| | | Blon_0113 | protein UreF |

The presence/absence of a B. longum infantis marker gene, Blon_0915, and 15 B. longum genes involved in human milk oligosaccharide (HMO) metabolism (LoCascio et al. 2010) were determined. 25/27 TA samples detected the marker gene and all 15 HMO genes, with correspondingly high copies per million (CPM) for most genes. By contrast, only 8 non-TA (5 farm and 3 nonfarm) detected Blon_0915. Six HMO genes were detected widely across the non-TA samples, while 9 were conspicuously absent from most. The latter nine were previously identified as uniquely and specifically conserved among infantis subspecies compared to other longum (LoCascio et al. 2010).

### Developing a Microbial Signature for Farm Groups

In addition to B. longum, other microbial taxa and functional pathways were identified that could distinguish the TA from non-TA microbiota. Multiple approaches were used: statistical comparison of microbial abundances and functional pathways, and training machine learning models followed by variable importance ranking.

### Machine Learning Models can Discriminate Between TA and Non-TA Samples

A suite of machine learning approaches was used to attempt to build classifiers to separate the TA from non-TA samples, and to identify important features. All algorithms achieved some success, with PR-AUC well above random guessing in all ten folds of cross-validation. The top performing algorithm was elastic net (implemented in glmnet), with mean PR-AUC=0.91. Two random forest implementations and linear support vector machines essentially tied for second place. The features employed by the glmnet and random forest classifiers to discriminate between the farm groups were examined (elastic net in FIG. 19). The elastic net approach also provides a sign on each feature that indicates which class (TA or non-TA) the feature is positively correlated to. The top features used by both algorithms were highly concordant, with 15 features appearing in the top 25 of both lists: (species) s_Actinomyces_sp_oral_taxon_181, s_Bacteroides_faecis, s__Bacteroides_stercoris, s__Bifidobacterium_bifidum, s_Bifidobacterium_longum, s_Bifidobacterium_pseudocatenulatum, s_Bilophila_wadsworthia, s__Collinsella_aerofaciens, s__Enterococcus_avium, s__Enterococcus_durans, s__Haemophilus_parainfluenzae, s__Parabacteroides_merdae, s_Streptococcus_peroris, s_Streptococcus_salivarius. Different members of the same genera, for examples Bacteroides and Bifidobacterium, were associated by glmnet with either TA or non-TA, suggesting that species-level (and perhaps more granular) genetic diversity varies between the groups.

### Differentially Abundant Microbes

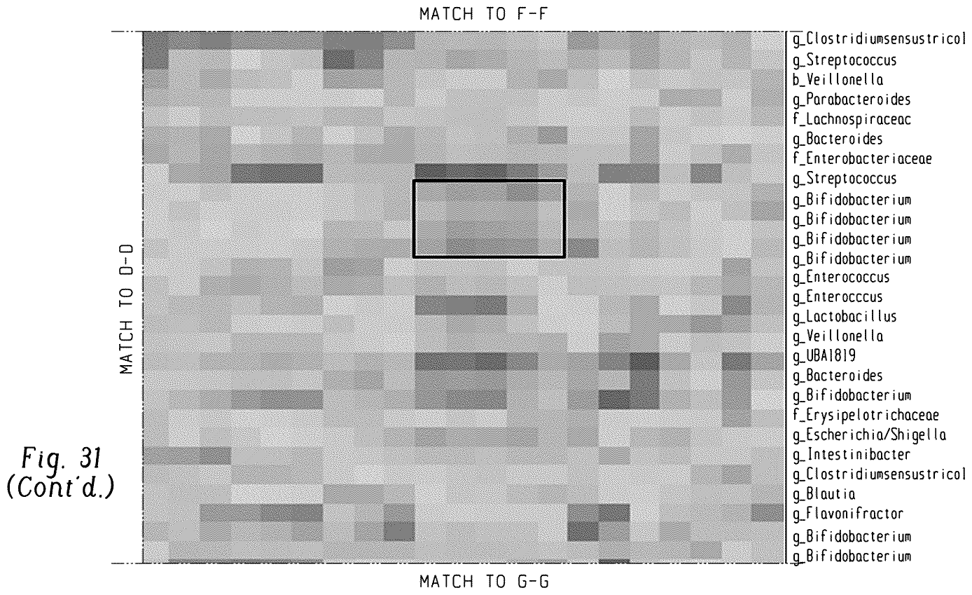

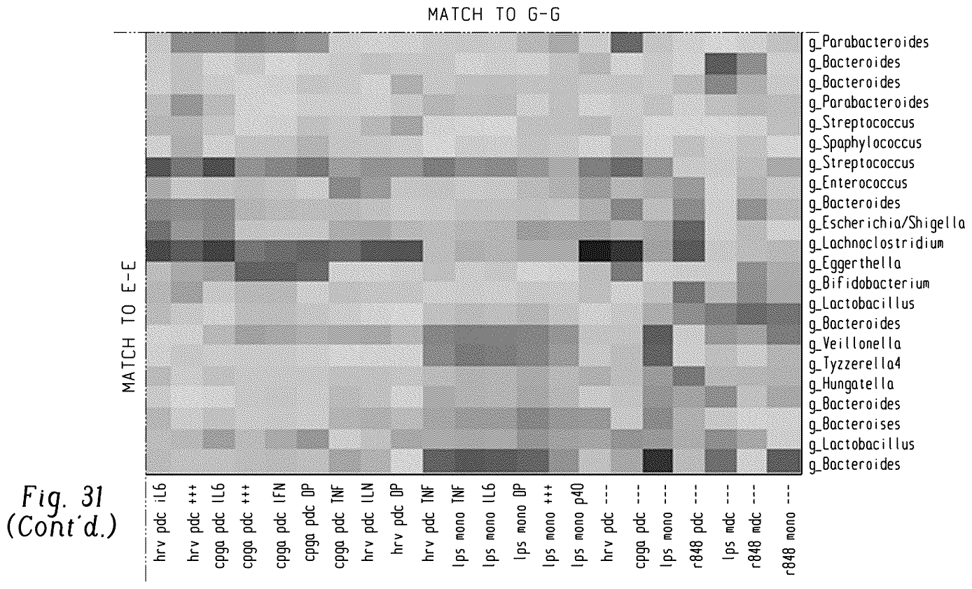

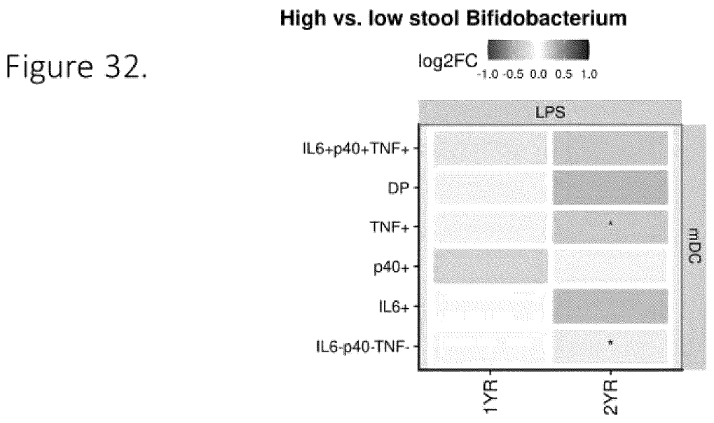

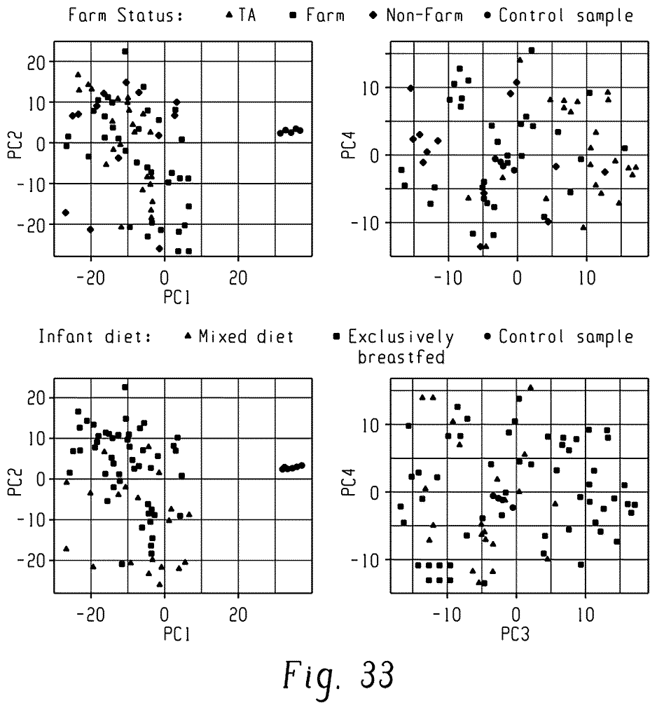

As a companion to the machine learning variable importance analysis, statistical tests were used to identify differentially abundant microbes between the groups. Non-parametric analysis by LEfSE (Segata et al. 2011) identified several taxa that were higher in TA compared to non-TA (FIGS. 32, 33), including a substantial overlap with the top features from the machine learning analysis.

### Differentially Abundant Functional Pathways

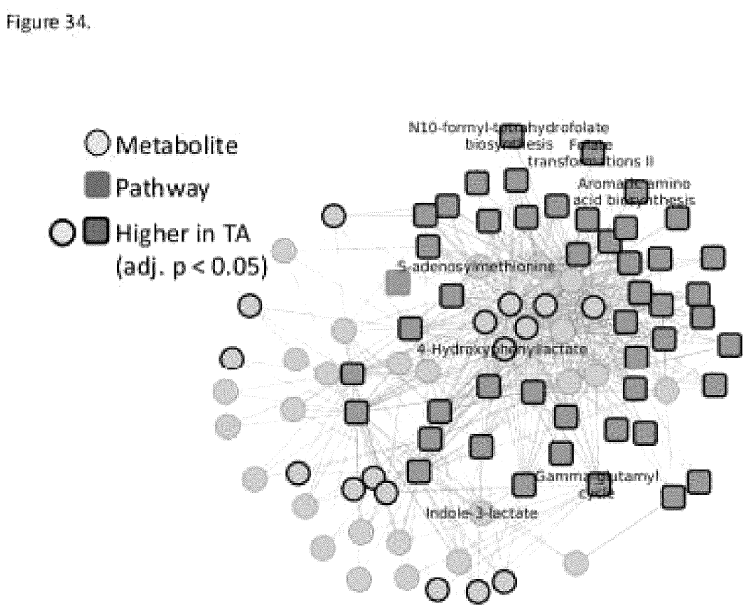

Differentially abundant MetaCyc™ pathways between TA and non-TA are shown in FIG. 34. Values shown are row-scaled loglp(copies per million). Pathway p<0.01 after Benjamini-Hochberg correction. Row annotations: coefficient (positive: higher in TA), negative log 10(adjusted p-value).

### Example 2

In one embodiment, for genus-level Bifidobacterium, if the total Bifidobacterium >80%, then there is a reduced disease risk and if the total Bifidobacterium <58%, then there is an increased disease risk.

TABLE 3

| | | non-TA | |
|---|---|---|---|
| | | (exclusive | |
| | | breastfeeding | non-TA |
| Genus | TA | only) | (all diets) |
| Bifidobacterium | 0.829 | 0.649 | 0.579 |

For species and subspecies level Bifidobacterium longum, in one embodiment, the Bifidobacterium longum subsp. infantis >71% and/or non-Bifidobacterium genera <17%, then there is a reduced disease risk while if the Bifidobacterium longum (any subspecies)<22% and/or total non-Bifidobacterium genera relative abundance >42%, then there is an increased disease risk.

TABLE 4

| | | non-TA | |
|---|---|---|---|
| | | (exclusive | |
| | | breastfeeding | non-TA, |
| Bifidobacterium Species | TA | only) | all STDs |
| Bifidobacterium_longum | 0.713 | 0.265 | 0.219 |
| Other (not | 0.171 | 0.351 | 0.421 |
| Bifidobacterium) | | | |

Diversity metrics are summaries of the distributions of the relative abundances, where higher “diversity” means more species are represented with more abundance, while higher “dominance” means fewer species have most of the abundance.

The following table shows exemplary means per group (all diets):

TABLE 5

| | | ‘non- | |
|---|---|---|---|
| metric | TA | TA’ | Direction |
| diversity_inverse_simpson (1/(sum of | 1.87 | 3.19 | TA < non- |
| squared relative abundances) | | | TA |
| diversity_coverage (number of species | 1.04 | 1.57 | TA < non- |
| needed to sum up to at least 50% of | | | TA |
| the relative abundance) | | | |
| diversity_gini_simpson ((1-(sum | 0.394 | 0.606 | TA < non- |
| of squared relative abundances)) | | | TA |
| dominance_relative (relative abundance | 0.74 | 0.534 | TA > non- |
| of the single most abundant taxon in | | | TA |
| each sample) | | | |
| dominance_core_abundance (combined | 0.736 | 0.257 | TA > non- |
| relative abundance of taxa that appear | | | TA |
| in at least half of the samples) | | | |

Metrics higher in non-TA (meaning more diversity, which implies B longum infantis is not dominant):
If inverse simpson alpha diversity >3, then increased risk
If inverse simpson alpha diversity <1.9, then decreased risk
If coverage diversity >1.5, then increased risk
If coverage diversity <1.04 then decreased risk
If gini simpson diversity >0.61, then increased risk
If gini simpson diversity <0.39, then decreased risk
   Metrics higher in TA than non-TA:
If dominance relative abundance (relative abundance of single most abundant taxon) >74%, then decreased risk
If dominance relative abundance <53%, then increased risk
If dominance core abundance <74%, then decreased risk
If dominance core abundance <26%, then increased risk

### Example 3

A table of relative abundances for the top features from machine learning analysis (FIG. 13). The purple ones are more associated with TA (decreased risk) and the yellow ones are more associated with non-TA (increased risk). The other tables in the figure contain the full list of top genera and Bifido species. A threshold of 1% relative abundance was set in the associated group. The top 5 in the “TA” set are shown in purple and the top 4 in the “non-TA” set are shown in yellow.

TABLE 6
Top learning machine features

| Associated | | | | non-TA | | | | | | |
|---|---|---|---|---|---|---|---|---|---|---|
| class (based on | | | non-TA | relative | | | | | | |
| centered log ratio | | TA | relative | abundance | | | mann | | glmnet | rank |
| transformed | | relative | abundance | (breast | glmnet | ranger | whitney- | | coefficient | correlation |
| data (CLR)) | Species | abundance | (all infants) | feeding only) | score | score | log10(p) | full clade_name | sign | on CLR |
| TA | s_Bifidobacterium_ | 0.7134 | 0.2186 | 0.2650 | 0.896 | 4.335 | 4.098 | k_Bacteria\|p_Actinobacteria\|c_Actinobacteria\| | TA | 0.5641155 |
| | longum | | | | | | | o_Bifidobacteriales\|f_Bifidobacteriaceae\| | | |
| | | | | | | | | g_Bifidobacterium\|s_Bifidobacterium_lonqum | | |
| TA | s_Bifidobacterium_ | 0.0808 | 0.0943 | 0.0942 | 0.472 | 0.969 | NA | k_Bacteria\|p_Actinobacteria\|c_Actinobacteria\| | TA | 0.257873256 |
| | bifidum | | | | | | | o_Bifidobacteriales\|f_Bifidobacteriaceae\| | | |
| | | | | | | | | g_Bifidobacterium\|s_Bifidobacterium_bifidum | | |
| TA | s_Bifidobacterium_ | 0.0336 | 0.1756 | 0.2050 | NA | 0.578 | NA | k_Bacteria\|p_Actinobacteria\|c_Actinobacteria\| | none | 0.068485119 |
| | breve | | | | | | | o_Bifidobacteriales\|f_Bifidobacteriaceae\| | | |
| | | | | | | | | g_Bifidobacterium\|s_Bifidobacterium_breve | | |
| TA | s_Parabacteroides_ | 0.0168 | 0.0213 | 0.0250 | NA | 0.324 | NA | k_Bacteria\|p_Bacteroidetes\|c_Bacteroidia\| | none | 0.152581114 |
| | distasonis | | | | | | | o_Bacteroidales\|f_Tannerellaceae\| | | |
| | | | | | | | | g_Parabacteroides\|s_Parabacteroides_distasonis | | |
| TA | s_Collinsella_ | 0.0103 | 0.0147 | 0.0114 | 0.391 | 0.488 | 1.312 | k_Bacteria\|p_Actinobacteria\|c_Coriobacteriia\| | TA | 0.333176874 |
| | aerofaciens | | | | | | | o_Coriobacteriales\|f_Coriobacteriaceae\| | | |
| | | | | | | | | g_Collinsella\|s_Collinsella_aerofaciens | | |
| TA | s_Bacteroides_ | 0.0069 | 0.0004 | 0.0005 | 0.246 | 0.595 | 1.312 | k_Bacteria\|p_Bacteroidetes\|c_Bacteroidia\| | TA | 0.338732633 |
| | faecis | | | | | | | o_Bacteroidales\|f_Bacteroidaceae\| | | |
| | | | | | | | | g_Bacteroides\|s_Bacteroides_faecis | | |
| TA | s_Parabacteroides_ | 0.0037 | 0.0025 | 0.0009 | 0.221 | 0.491 | NA | k_Bacteria\|p_Bacteroidetes\|c_Bacteroidia\| | TA | 0.30111717 |
| | merdae | | | | | | | o_Bacteroidales\|f_Tannerellaceae\| | | |
| | | | | | | | | g_Parabacteroides\|s_Parabacteroides_merdae | | |
| TA | s_Bacteroides_ | 0.0027 | 0.0013 | 0.0009 | 0.559 | 0.658 | NA | k_Bacteria\|p_Bacteroidetes\|c_Bacteroidia\| | TA | 0.310929705 |
| | stercoris | | | | | | | o_Bacteroidales\|f_Bacteroidaceae\| | | |
| | | | | | | | | g_Bacteroides\|s_Bacteroides_stercoris | | |
| TA | s_Enterococcus_ | 0.0016 | 0.0016 | 0.0002 | 1.127 | 1.194 | 1.312 | k_Bacteria\|p_Firmicutes\|c_Bacilli\| | TA | 0.325467951 |
| | avium | | | | | | | o_Lactobacillales\|f_Enterococcaceae\| | | |
| | | | | | | | | g_Enterococcus\|s_Enterococcus_avium | | |
| TA | s_Bifidobacterium_ | 0.0011 | 0.0156 | 0.0248 | NA | 0.266 | NA | k_Bacteria\|p_Actinobacteria\|c_Actinobacteria\| | none | 0.265321611 |
| | dentium | | | | | | | o_Bifidobacteriales\|f_Bifidobacteriaceae\| | | |
| | | | | | | | | g_Bifidobacterium\|s_Bifidobacterium_dentium | | |
| TA | s_Lactobacillus_ | 0.0008 | 0.0003 | 0.0004 | 0.282 | NA | NA | k_Bacteria\|p_Firmicutes\|c_Bacilli\| | TA | −0.035738222 |
| | gasseri | | | | | | | o_Lactobacillales\|f_Lactobacillaceae\| | | |
| | | | | | | | | g_Lactobacillus\|s_Lactobacillus_gasseri | | |
| TA | s_Enterococcus_ | 0.0007 | 0.0008 | 0.0000 | 0.312 | 0.865 | 1.617 | k_Bacteria\|p_Firmicutes\|c_Bacilli\| | TA | 0.377012091 |
| | durans | | | | | | | o_Lactobacillales\|f_Enterococcaceae\| | | |
| | | | | | | | | g_Enterococcus\|s_Enterococcus_durans | | |
| TA | s_Bacteroides_ | 0.0007 | 0.0010 | 0.0002 | NA | 0.284 | NA | k_Bacteria\|p_Bacteroidetes\|c_Bacteroidia\| | none | 0.054401947 |
| | ovatus | | | | | | | o_Bacteroidales\|f_Bacteroidaceae\| | | |
| | | | | | | | | g_Bacteroides\|s_Bacteroides_ovatus | | |
| TA | s_Streptococcus_ | 0.0006 | 0.0016 | 0.0026 | NA | 0.260 | NA | k_Bacteria\|p_Firmicutes\|c_Bacilli\| | none | 0.007843237 |
| | mitis | | | | | | | o_Lactobacillales\|f_Streptococcaceae\| | | |
| | | | | | | | | g_Streptococcus\|s_Streptococcus_mitis | | |
| TA | s_Actinomyces_sp_ | 0.0005 | 0.0000 | 0.0000 | 0.636 | 3.139 | 3.359 | k_Bacteria\|p_Actinobacteria\|c_Actinobacteria\| | TA | 0.510650315 |
| | oral_taxon_181 | | | | | | | o_Actinomycetales\|f_Actinomycetaceae\| | | |
| | | | | | | | | g_Actinomyces\|s_Actinomyces_sp_oral_taxon_181 | | |
| TA | s_Bilophila_ | 0.0004 | 0.0001 | 0.0001 | 0.361 | 0.364 | NA | k_Bacteria\|p_Proteobacteria\|c_ | TA | 0.201570209 |
| | wadsworthia | | | | | | | Deltaproteobacteria\|o_Desulfovibrionales\|f_ | | |
| | | | | | | | | Desulfovibrionaceae\|g_Bilophila\|s_Bilophila_wadsworthia | | |
| TA | s_Streptococcus_ | 0.0003 | 0.0001 | 0.0001 | 0.980 | 1.653 | 2.325 | k_Bacteria\|p_Firmicutes\|c_Bacilli\| | TA | 0.440807168 |
| | peroris | | | | | | | o_Lactobacillales\|f_Streptococcaceae\| | | |
| | | | | | | | | g_Streptococcus\|s_Streptococcus_peroris | | |
| TA | s_Anaerococcus_ | 0.0002 | 0.0000 | 0.0000 | NA | 0.587 | 1.312 | k_Bacteria\|p_Firmicutes\|c_Tissierellia\| | none | 0.336679708 |
| | vaginalis | | | | | | | o_Tissierellales\|f_Peptoniphilaceae\| | | |
| | | | | | | | | g_Anaerococcus\|s_Anaerococcus_vaginalis | | |
| TA | s_Actinomyces_ | 0.0001 | 0.0002 | 0.0003 | 0.355 | NA | NA | k_Bacteria\|p_Actinobacteria\|c_Actinobacteria\| | TA | 0.122690145 |
| | odontolyticus | | | | | | | o_Actinomycetales\|f_Actinomycetaceae\| | | |
| | | | | | | | | g_Actinomyces\|s_Actinomyces_odontolyticus | | |
| TA | s_['Collinsella]_ | 0.0000 | 0.0000 | 0.0000 | 0.361 | NA | NA | k_Bacteria\|p_Actinobacteria\|c_Coriobacteriia\| | TA | 0.252048516 |
| | massiliensis | | | | | | | o_Coriobacteriales\|f_Coriobacteriaceae\| | | |
| | | | | | | | | g_Enorma\|s_[Collinsella]_massiliensis | | |
| TA | s_Streptococcus_ | 0.0000 | 0.0001 | 0.0002 | 0.209 | NA | NA | k_Bacteria\|p_Firmicutes\|c_Bacilli\| | TA | 0.044638423 |
| | infantis | | | | | | | o_Lactobacillales\|f_Streptococcaceae\| | | |
| | | | | | | | | g_Streptococcus\|s_Streptococcus_infantis | | |
| TA | s_Finegoldia_ | 0.0000 | 0.0000 | 0.0000 | NA | 0.271 | NA | k_Bacteria\|p_Firmicutes\|c_Tissierellia\| | none | 0.196569753 |
| | magna | | | | | | | o_Tissierellales\|f_Peptoniphilaceae\| | | |
| | | | | | | | | g_Finegoldia\|s_Finegoldia_magna | | |
| TA | s_Streptococcus_ | 0.0000 | 0.0000 | 0.0000 | 0.405 | NA | NA | k_Bacteria\|p_Firmicutes\|c_Bacilli\| | TA | 0.219531894 |
| | sp_HMSC071D03 | | | | | | | o_Lactobacillales\|f_Streptococcaceae\| | | |
| | | | | | | | | g_Streptococcus\|s_Streptococcus_sp_HMSC071D03 | | |
| non-TA | s_Bifidobacterium_ | 0.0000 | 0.0408 | 0.0336 | 0.948 | 0.469 | 1.617 | k_Bacteria\|p_Actinobacteria\|c_Actinobacteria\| | non- | −0.374039199 |
| | pseudocatenulatum | | | | | | | o_Bifidobacteriales\|f_Bifidobacteriaceae\| | TA | |
| | | | | | | | | g_Bifidobacterium\|s_Bifidobacterium_pseudocatenulatum | | |
| non-TA | s_Escherichia_coli | 0.0222 | 0.0386 | 0.0494 | 0.543 | NA | NA | k_Bacteria\|p_Proteobacteria\|c_ | non- | 0.041550877 |
| | | | | | | | | Gammaproteobacteria\|o_Enterobacterales\|f_ | TA | |
| | | | | | | | | Enterobacteriaceae\|g_Escherichia\|s_Escherichia_coli | | |
| non-TA | s_Bacteroides_ | 0.0008 | 0.0242 | 0.0286 | 0.235 | NA | NA | k_Bacteria\|p_Bacteroidetes\|c_Bacteroidia\| | non- | −0.190106231 |
| | thetaiotaomicron | | | | | | | o_Bacteroidales\|f_Bacteroidaceae\| | TA | |
| | | | | | | | | g_Bacteroides\|s_Bacteroides_thetaiotaomicron | | |
| non-TA | s_Bifidobacterium_ | 0.0000 | 0.0201 | 0.0104 | 0.499 | NA | NA | k_Bacteria\|p_Actinobacteria\|c_Actinobacteria\| | non- | −0.264614336 |
| | adolescentis | | | | | | | o_Bifidobacteriales\|f_Bifidobacteriaceae\| | TA | |
| | | | | | | | | g_Bifidobacterium\|s_Bifidobacterium_adolescentis | | |
| non-TA | s_Klebsiella_ | 0.0000 | 0.0079 | 0.0128 | 0.418 | NA | NA | k_Bacteria\|p_Proteobacteria\|c_ | non- | −0.279322423 |
| | michiganensis | | | | | | | Gammaproteobacteria\|o_Enterobacterales\|f_ | TA | |
| | | | | | | | | Enterobacteriaceae\|g_Klebsiella\|s_Klebsiella_michiganensis | | |
| non-TA | s_Clostridium_ | 0.0075 | 0.0060 | 0.0094 | NA | 0.303 | NA | k_Bacteria\|p_Firmicutes\|c_Clostridia\| | none | −0.130726656 |
| | neonatale | | | | | | | o_Clostridiales\|f_Clostridiaceae\| | | |
| | | | | | | | | g_Clostridium\|s_Clostridium_neonatale | | |
| non-TA | s_Streptococcus_ | 0.0011 | 0.0043 | 0.0042 | 0.673 | 1.397 | 1.312 | k_Bacteria\|p_Firmicutes\|c_Bacilli\| | non- | −0.326060035 |
| | salivarius | | | | | | | o_Lactobacillales\|f_Streptococcaceae\| | TA | |
| | | | | | | | | g_Streptococcus\|s_Streptococcus_salivarius | | |
| non-TA | s_Streptococcus_ | 0.0003 | 0.0016 | 0.0018 | NA | 0.517 | NA | k_Bacteria\|p_Firmicutes\|c_Bacilli\| | none | −0.12389155 |
| | parasanguinis | | | | | | | o_Lactobacillales\|f_Streptococcaceae\| | | |
| | | | | | | | | g_Streptococcus\|s_Streptococcus_parasanguinis | | |
| non-TA | s_Staphylococcus_ | 0.0003 | 0.0012 | 0.0011 | NA | 0.400 | NA | k_Bacteria\|p_Firmicutes\|c_Bacilli\| | none | −0.247687847 |
| | epidermidis | | | | | | | o_Bacillales\|f_Staphylococcaceae\| | | |
| | | | | | | | | g_Staphylococcus\|s_Staphylococcus_epidermidis | | |
| non-TA | s_Veillonella_ | 0.0000 | 0.0010 | 0.0013 | NA | 0.390 | 1.312 | k_Bacteria\|p_Firmicutes\|c_Negativicutes\| | none | −0.326597126 |
| | dispar | | | | | | | o_Veillonellales\|f_Veillonellaceae\| | | |
| | | | | | | | | g_Veillonella\|s_Veillonella_dispar | | |
| non-TA | s_Haemophilus_ | 0.0000 | 0.0004 | 0.0006 | 0.828 | 0.742 | 1.957 | k_Bacteria\|p_Proteobacteria\|c_ | non- | −0.409352834 |
| | parainfluenzae | | | | | | | Gammaproteobacteria\|o_Pasteurellales\|f_ | TA | |
| | | | | | | | | Pasteurellaceae\|g_Haemophilus\|s_ | | |
| | | | | | | | | Haemophilus_parainfluenzae | | |
| non-TA | s_Cutibacterium_ | 0.0000 | 0.0003 | 0.0005 | 0.239 | NA | NA | k_Bacteria\|p_Actinobacteria\|c_Actinobacteria\| | non- | −0.087249315 |
| | avidum | | | | | | | o_Propionibacteriales\|f_Propionibacteriaceae\| | TA | |
| | | | | | | | | g_Cutibacterium\|s_Cutibacterium_avidum | | |
| non-TA | s_Veillonella_sp_ | 0.0000 | 0.0000 | 0.0001 | 0.275 | NA | 1.312 | k_Bacteria\|p_Firmicutes\|c_Negativicutes\| | non- | −0.336322381 |
| | T11011_6 | | | | | | | o_Veillonellales\|f_Veillonellaceae\| | TA | |
| | | | | | | | | g_Veillonella\|s_Veillonella_sp_T11011_6 | | |

TABLE 7
Top species

| | | | non-TA |
|---|---|---|---|
| | | | (exclusive |
| | | non-TA | breastfeeding |
| Species | TA | (all diets) | only) |
| Bifidobacterium_longum | 0.713 | 0.219 | 0.265 |
| Bifidobacterium_bifidum | 0.081 | 0.094 | 0.094 |
| Bifidobacterium_breve | 0.034 | 0.176 | 0.205 |
| Escherichia_coli | 0.022 | 0.039 | 0.049 |
| Bacteroides_fragilis | 0.020 | 0.022 | 0.028 |
| Parabacteroides_distasonis | 0.017 | 0.021 | 0.025 |
| Other | 0.016 | 0.071 | 0.052 |
| Bacteroides_vulgatus | 0.015 | 0.022 | 0.018 |
| Bacteroides_dorei | 0.011 | 0.011 | 0.011 |
| Collinsella_aerofaciens | 0.010 | 0.015 | 0.011 |
| Clostridium_neonatale | 0.008 | 0.006 | 0.009 |
| Klebsiella_oxytoca | 0.007 | 0.007 | 0.006 |
| Lactobacillus_rhamnosus | 0.006 | 0.004 | 0.005 |
| Enterococcus_faecalis | 0.004 | 0.005 | 0.005 |
| Parabacteroides_merdae | 0.004 | 0.003 | 0.001 |
| Veillonella_parvula | 0.004 | 0.005 | 0.001 |
| Eggerthella_lenta | 0.003 | 0.002 | 0.001 |
| Bacteroides_stercoris | 0.003 | 0.001 | 0.001 |
| Erysipelatoclostridium_ramosum | 0.003 | 0.023 | 0.015 |
| Bacteroides_caccae | 0.003 | 0.007 | 0.009 |
| Ruminococcus_gnavus | 0.002 | 0.054 | 0.023 |
| Enterococcus_avium | 0.002 | 0.002 | 0.000 |
| Bacteroides_uniformis | 0.001 | 0.004 | 0.003 |
| Bifidobacterium_dentium | 0.001 | 0.016 | 0.025 |
| Streptococcus_salivarius | 0.001 | 0.004 | 0.004 |
| Veillonella_atypica | 0.001 | 0.002 | 0.001 |
| Collinsella_stercoris | 0.001 | 0.001 | 0.001 |
| Lactobacillus_gasseri | 0.001 | 0.000 | 0.000 |
| Bacteroides_thetaiotaomicron | 0.001 | 0.024 | 0.029 |
| Flavonifractor_plautii | 0.001 | 0.004 | 0.002 |
| Enterococcus_durans | 0.001 | 0.001 | 0.000 |
| Bacteroides_ovatus | 0.001 | 0.001 | 0.000 |
| Klebsiella_pneumoniae | 0.001 | 0.009 | 0.010 |
| Streptococcus_mitis | 0.001 | 0.002 | 0.003 |
| Actinomyces_sp_oral_taxon_181 | 0.000 | 0.000 | 0.000 |
| Streptococcus_vestibularis | 0.000 | 0.001 | 0.000 |
| Gordonibacter_pamelaeae | 0.000 | 0.001 | 0.000 |
| Bilophila_wadsworthia | 0.000 | 0.000 | 0.000 |
| Klebsiella_variicola | 0.000 | 0.005 | 0.007 |
| Streptococcus_parasanguinis | 0.000 | 0.002 | 0.002 |
| Staphylococcus_aureus | 0.000 | 0.000 | 0.000 |
| Bacteroides_xylanisolvens | 0.000 | 0.001 | 0.000 |
| Streptococcus_peroris | 0.000 | 0.000 | 0.000 |
| Staphylococcus_epidermidis | 0.000 | 0.001 | 0.001 |
| Clostridium_clostridioforme | 0.000 | 0.001 | 0.000 |
| Clostridium_innocuum | 0.000 | 0.005 | 0.003 |
| Actinomyces_odontolyticus | 0.000 | 0.000 | 0.000 |
| Lactobacillus_paragasseri | 0.000 | 0.002 | 0.001 |
| Enterococcus_faecium | 0.000 | 0.001 | 0.000 |
| Intestinibacter_bartlettii | 0.000 | 0.001 | 0.000 |
| Actinomyces_sp_HPA0247 | 0.000 | 0.000 | 0.000 |
| Klebsiella_quasipneumoniae | 0.000 | 0.001 | 0.001 |
| Enterococcus_gallinarum | 0.000 | 0.003 | 0.000 |
| Rothia_mucilaginosa | 0.000 | 0.000 | 0.000 |
| Clostridium_paraputrificum | 0.000 | 0.001 | 0.000 |
| Clostridium_butyricum | 0.000 | 0.002 | 0.002 |
| Veillonella_dispar | 0.000 | 0.001 | 0.001 |
| Enterobacter_cloacae_complex | 0.000 | 0.000 | 0.000 |
| Clostridium_perfringens | 0.000 | 0.003 | 0.004 |
| Veillonella_infantium | 0.000 | 0.000 | 0.000 |
| Streptococcus_thermophilus | 0.000 | 0.000 | 0.000 |
| Haemophilus_parainfluenzae | 0.000 | 0.000 | 0.001 |
| Bifidobacterium_adolescentis | 0.000 | 0.020 | 0.010 |
| Bifidobacterium_pseudocatenulatum | 0.000 | 0.041 | 0.034 |
| Klebsiella_michiganensis | 0.000 | 0.008 | 0.013 |
| Streptococcus_lutetiensis | 0.000 | 0.023 | 0.002 |

TABLE 8
Relative abundance Bifidobacterium species

| | | non-TA | |
|---|---|---|---|
| | | (exclusive | |
| | | breastfeeding | non-TA, |
| Bifidobacterium Species | TA | only) | all SIDs |
| Bifidobacterium_longum | 0.713 | 0.265 | 0.219 |
| (includes infantis and other subspecies) | | | |
| Other (not Bifidobacterium) | 0.171 | 0.351 | 0.421 |
| Bifidobacterium_bifidum | 0.081 | 0.094 | 0.094 |
| Bifidobacterium_breve | 0.034 | 0.205 | 0.176 |
| Bifidobacterium_dentium | 0.001 | 0.025 | 0.016 |
| Bifidobacterium_adolescentis | 0.000 | 0.010 | 0.020 |
| Bifidobacterium_animalis | 0.000 | 0.000 | 0.000 |
| Bifidobacterium_anseris | 0.000 | 0.000 | 0.000 |
| Bifidobacterium_boum | 0.000 | 0.000 | 0.000 |
| Bifidobacterium_catenulatum | 0.000 | 0.000 | 0.000 |
| Bifidobacterium_choerinum | 0.000 | 0.000 | 0.000 |
| Bifidobacterium_criceti | 0.000 | 0.000 | 0.000 |
| Bifidobacterium_gallinarum | 0.000 | 0.000 | 0.000 |
| Bifidobacterium_kashiwanohense | 0.000 | 0.016 | 0.010 |
| Bifidobacterium_merycicum | 0.000 | 0.000 | 0.000 |
| Bifidobacterium_minimum | 0.000 | 0.000 | 0.000 |
| Bifidobacterium_mongoliense | 0.000 | 0.000 | 0.000 |
| Bifidobacterium_moukalabense | 0.000 | 0.000 | 0.000 |
| Bifidobacterium_pseudocatenulatum | 0.000 | 0.034 | 0.041 |
| Bifidobacterium_pseudolongum | 0.000 | 0.000 | 0.000 |
| Bifidobacterium_pullorum | 0.000 | 0.000 | 0.003 |
| Bifidobacterium_ruminantium | 0.000 | 0.000 | 0.000 |
| Bifidobacterium_saeculare | 0.000 | 0.000 | 0.000 |
| Bifidobacterium_scardovii | 0.000 | 0.000 | 0.000 |
| Bifidobacterium_subtile | 0.000 | 0.000 | 0.000 |
| Bifidobacterium_thermacidophilum | 0.000 | 0.000 | 0.000 |
| Bifidobacterium_thermophilum | 0.000 | 0.000 | 0.000 |

TABLE 9
Mean alpha diversities

| metric | TA | ‘non-TA’ | Direction |
|---|---|---|---|
| diversity_inverse_simpson | 1.87 | 3.19 | −1.32 |
| diversity_coverage | 1.04 | 1.57 | −0.53 |
| diversity_gini_simpson | 0.394 | 0.606 | −0.212 |
| dominance_relative | 0.74 | 0.534 | 0.206 |
| dominance_core_abundance | 0.736 | 0.257 | 0.479 |

TABLE 10
Relative abundance, top genera (1% in 10% samples)
Relative abundance, top genera (1% in 10% samples)

| | | non-TA | |
|---|---|---|---|
| | | (exclusive | |
| | | breastfeeding | non-TA |
| Genus | TA | only) | (ALL) |
| Bifidobacterium | 0.829 | 0.649 | 0.579 |
| Bacteroides | 0.063 | 0.101 | 0.096 |
| Escherichia | 0.022 | 0.049 | 0.039 |
| Parabacteroides | 0.021 | 0.026 | 0.024 |
| Collinsella | 0.011 | 0.013 | 0.016 |
| Clostridium | 0.008 | 0.018 | 0.013 |
| Klebsiella | 0.008 | 0.037 | 0.030 |
| Lactobacillus | 0.007 | 0.007 | 0.008 |
| Enterococcus | 0.006 | 0.006 | 0.012 |
| Other | 0.005 | 0.014 | 0.024 |
| Veillonella | 0.005 | 0.010 | 0.013 |
| Eggerthella | 0.003 | 0.001 | 0.002 |
| Streptococcus | 0.003 | 0.019 | 0.036 |
| Erysipelatoclostridium | 0.003 | 0.019 | 0.029 |
| Blautia | 0.002 | 0.023 | 0.066 |
| Actinomyces | 0.002 | 0.001 | 0.001 |
| Flavonifractor | 0.001 | 0.002 | 0.004 |
| Staphylococcus | 0.001 | 0.002 | 0.002 |
| Eubacterium | 0.000 | | 0.000 |
| Gordonibacter | 0.000 | 0.000 | 0.001 |
| Bilophila | 0.000 | 0.000 | 0.000 |
| Lachnoclostridium | 0.000 | 0.001 | 0.003 |
| Phascolarctobacterium | 0.000 | | 0.000 |
| Corynebacterium | 0.000 | 0.000 | 0.000 |
| Intestinibacter | 0.000 | | 0.001 |
| Rothia | 0.000 | 0.000 | 0.000 |
| Gemella | 0.000 | 0.001 | 0.001 |
| Enterobacter | 0.000 | | 0.000 |
| Citrobacter | 0.000 | | 0.001 |
| Haemophilus | 0.000 | 0.001 | 0.001 |

### Example 4

Thus, breastfeeding and traditional agrarian lifestyle influence 2-month-old infants' gut microbiome composition. TA infant gut is dominated by Bifidobacterium longum subspecies infantis. B infantis and early gut commensals are selected by breastmilk oligosaccharides to colonize, preventing colonization by more pathogenic bacteria and those bacteria have been shown to produce nutritive and anti-inflammatory metabolites.

B. infantis has a broad capacity to break down human milk oligosaccharides. B. infantis is declining in industrialized communities, but still found in agrarian communities. B infantis and potentially other early life gut commensals may influence healthy development that includes protecting against pathogen colonization, e.g., by producing nutritive and immunomodulatory molecules, e.g., B vitamins, short chain fatty acids (SCFAs, e.g., fatty acids with fewer than 6 carbons), folic acid and/or tryptophan metabolites. Bacterially produced aromatic amino acid metabolites and exopolypeptides have a tolerogenic effect on gut epithelial and T cells.

### Example 5

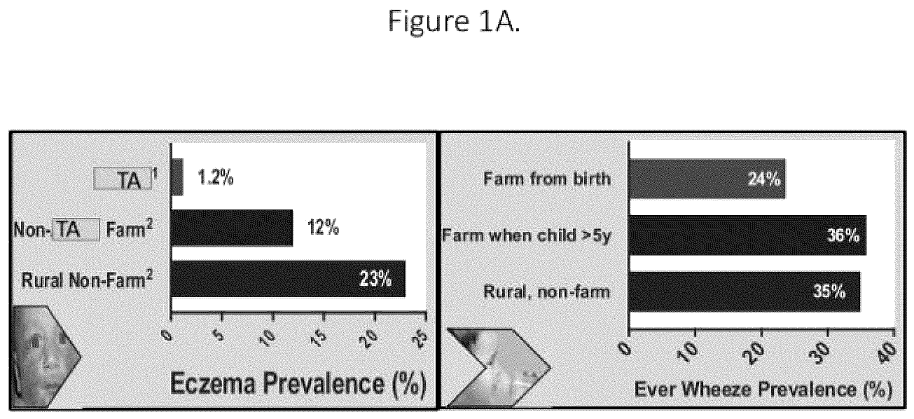

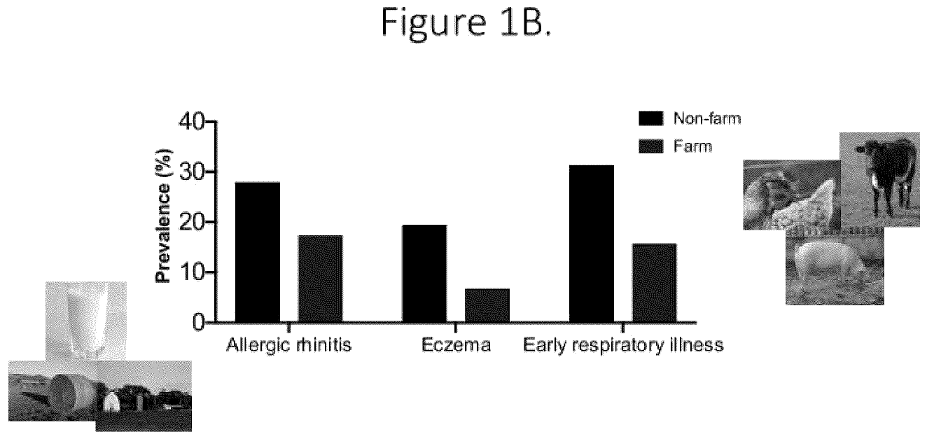

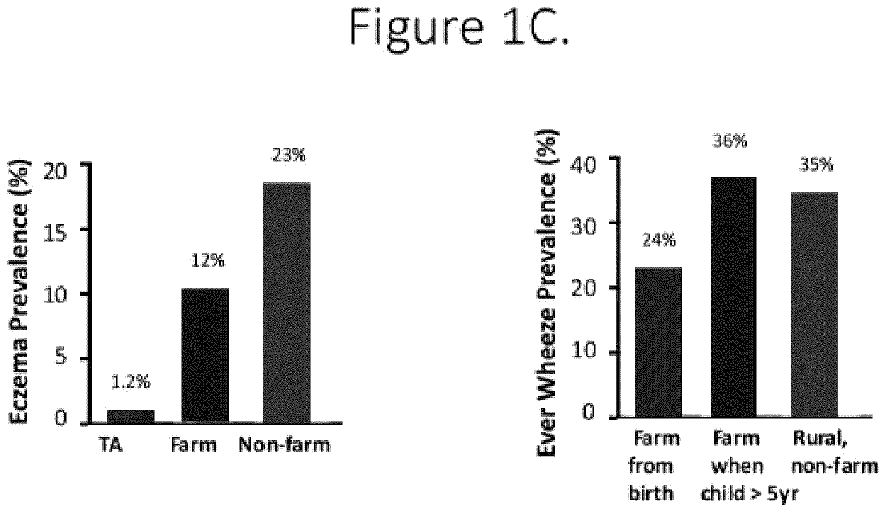

Asthma is an immune-mediated chronic illness, and its prevalence is increasing worldwide. It is a lifelong disease and treatment is primarily focused on symptom management. Development of asthma begins in very early life, but it is not diagnosed until later in childhood. It is often preceded by conditions including allergic rhinitis, eczema, and wheezing. People who grow up on farms have reduced rates of asthma and immune-mediated diseases. The histograms in FIG. 1 show data from a study conducted in Wisconsin. In red, children who grew up on farms have lower prevalence of these conditions compared to non-farm in black. Several maternal and infant lifestyle practices have been associated with protection against disease development. They include close contact with farm animals and their stables, especially by milking cows, and frequent ingestion of unpasteurized cow's milk.

Intriguingly, allergy prevalence is even lower in WI TA children compared to WI farm children (FIG. 1). In fact, eczema prevalence is 10 times lower in WI TA children compared to non-TA WI farm children. Also, children who moved to farms after the age of 5 did not enjoy the protective effects experienced by those who lived on farms from birth. Very early life exposure starting during pregnancy of animal and farm milk have the highest protection against allergic diseases.

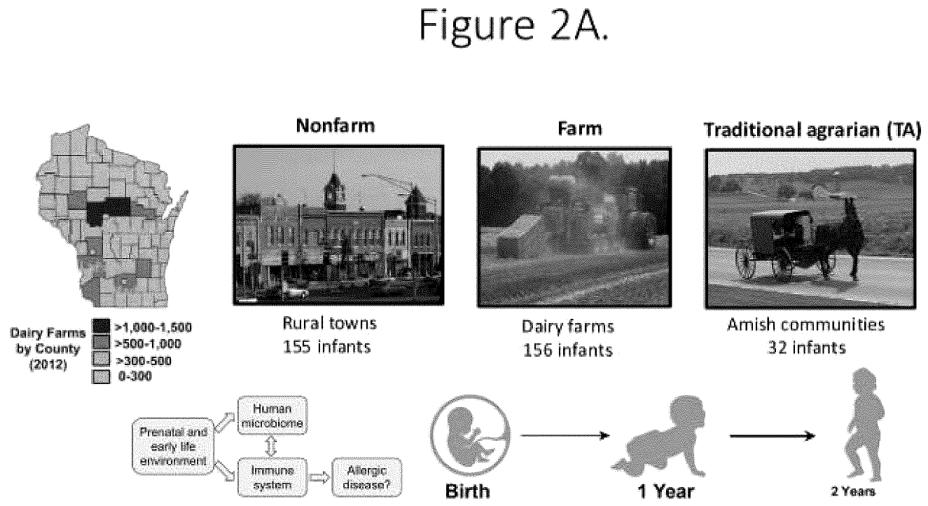

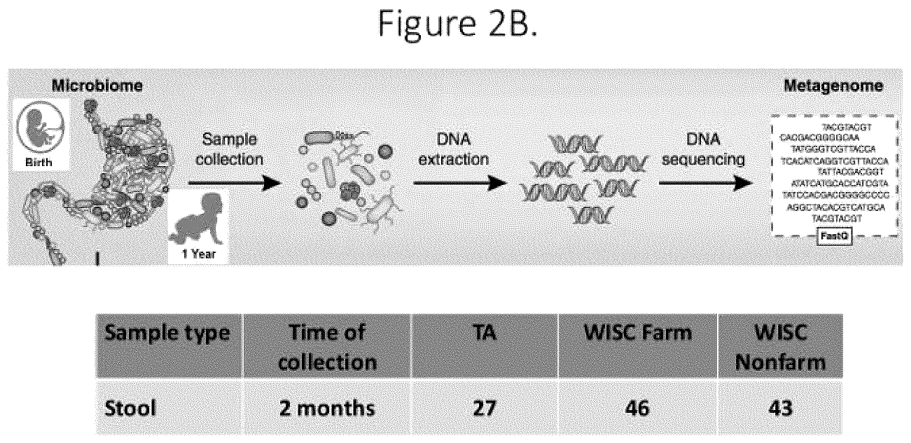

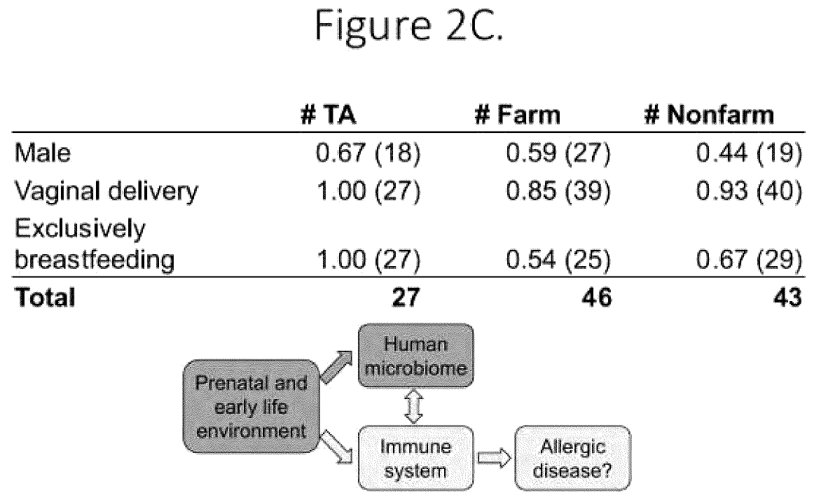

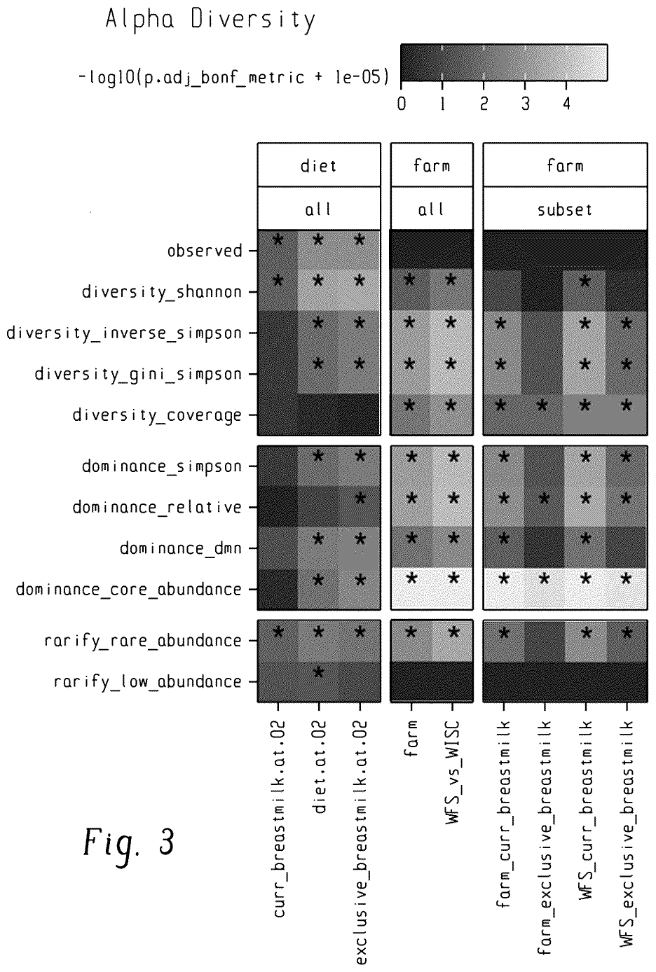

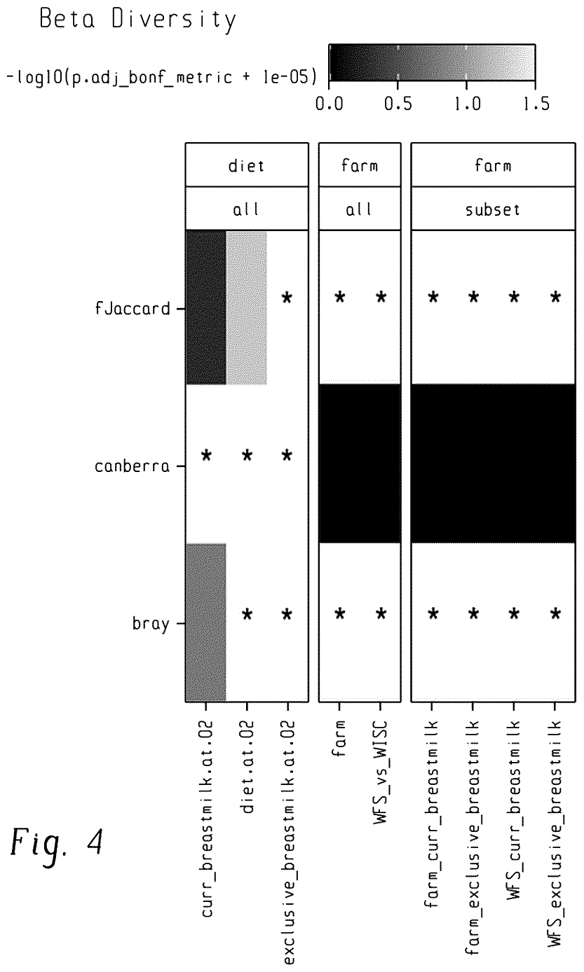

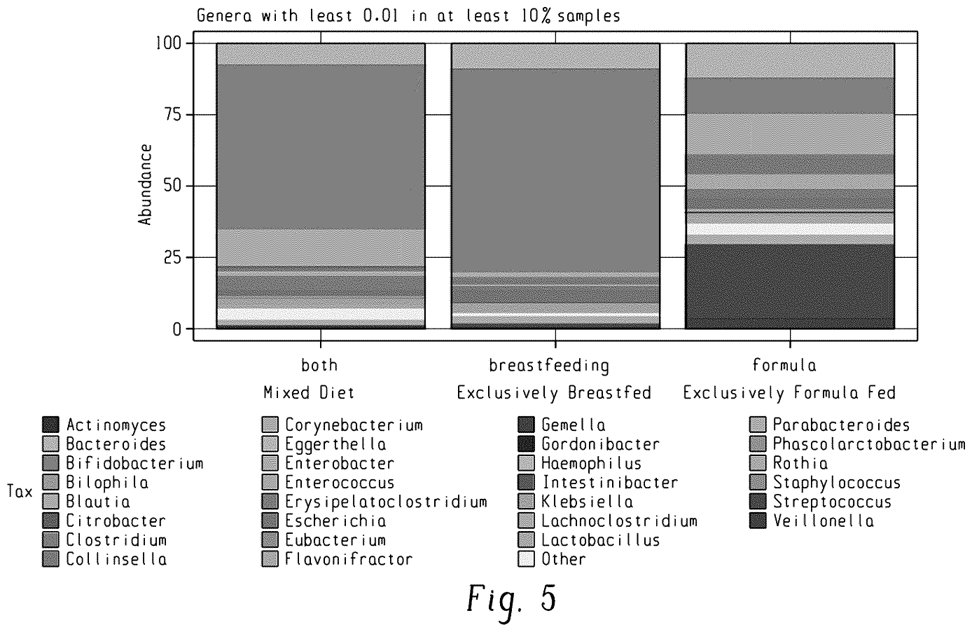

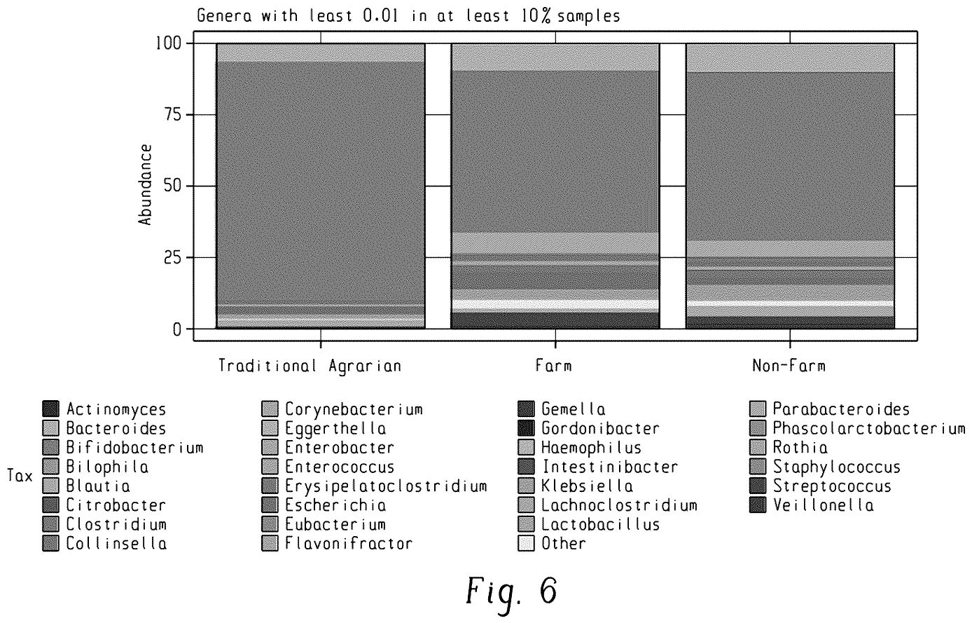

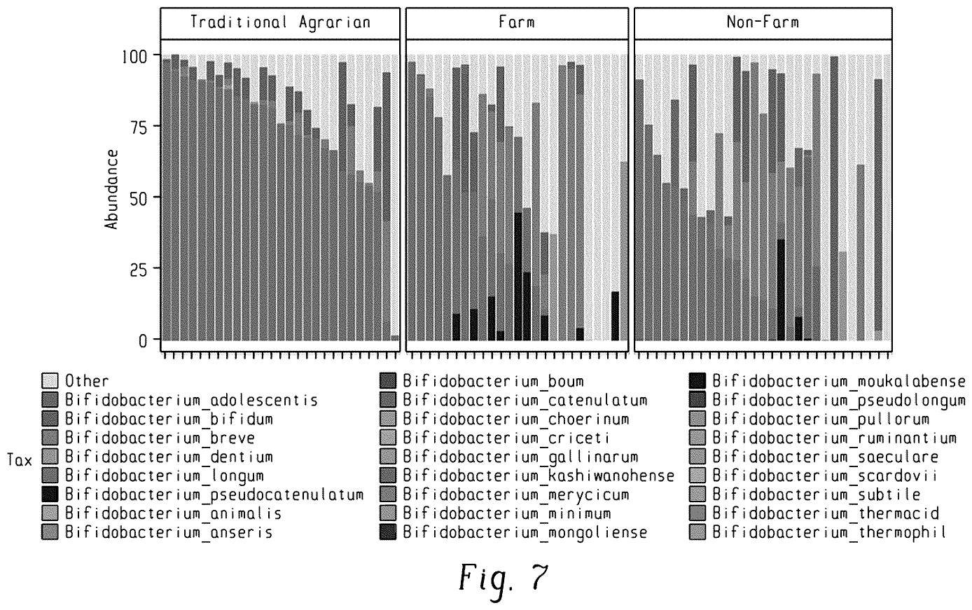

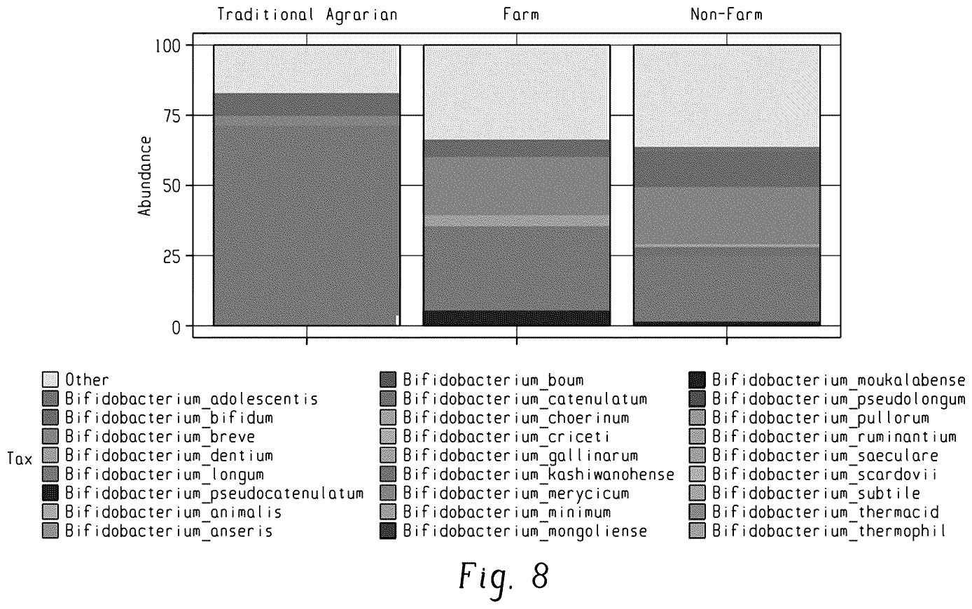

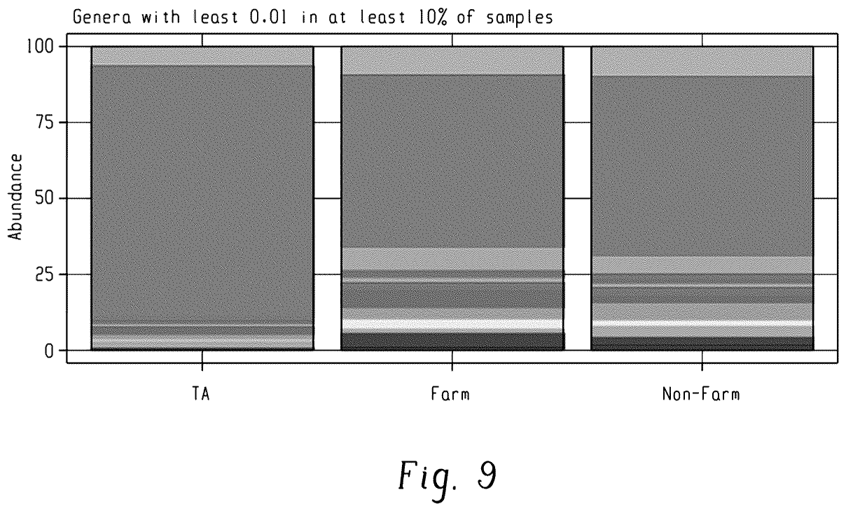

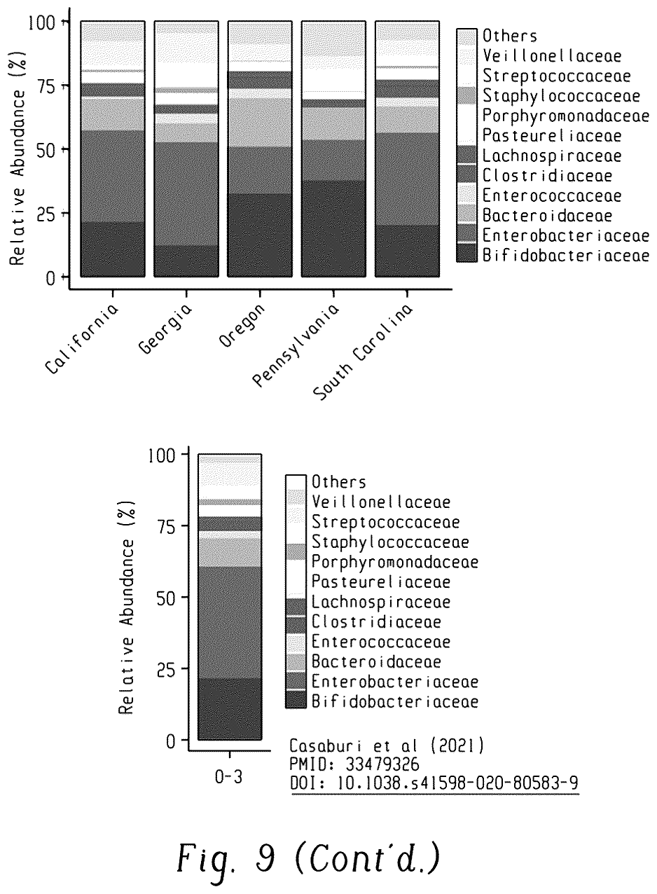

FIG. 2A illustrates the Wisconsin Infant Study Cohort (WISC) and Wisconsin Farm Study (WFS). The Wisconsin Infant Study Cohort (WISC) and Wisconsin Farm Study are prospective birth cohort studies that aim to identify molecular contributors of farm exposures on development of asthma and childhood respiratory illness. Together they consist of 3 arms: The Wisconsin Infant Study Cohort (WISC) includes infants from non-farming and dairy farming families in upper and central Wisconsin. Wisconsin Farm Study arm of the project is comprised of infants from Wisconsin TA families who follow a traditional agrarian lifestyle (“TA”). These studies recruited families who were expecting a child and followed the children through the first two years of life, collecting health information and a broad range of environmental and personal biospecimens.

The gut microbiomes at two months of age were compared between the farm exposure groups (FIG. 2C). Whole genome shotgun metagenomics sequencing was performed on 116 stool samples with participant characteristics shown in this table. All TA infants were delivered vaginally and are exclusively breastfed, so we enriched for those categories when we selected non-TA samples. It was hypothesized that the microbial communities of the groups would vary with the level of farming exposure, and that the TA infants would harbor unique microbes compared to the non-TA infants.

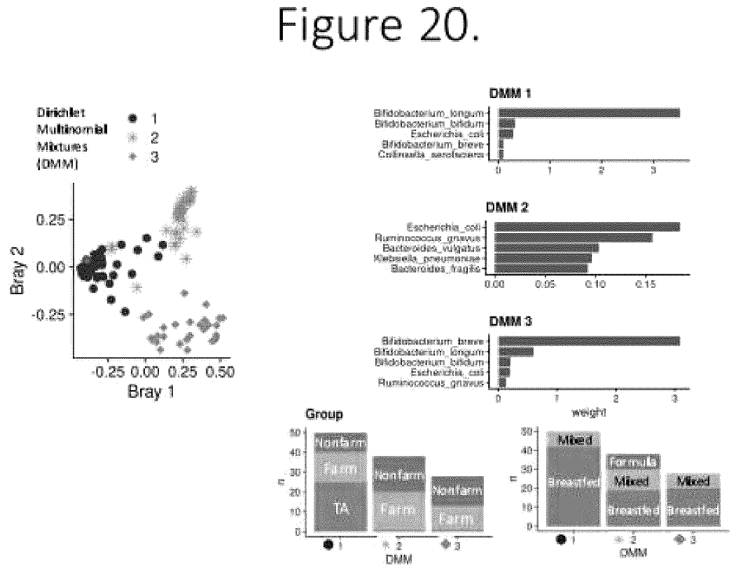

Beta diversity from species level features was computed using the Bray distance, and the samples clustered with Dirichlet Multinomial Mixtures to identify latent structure (FIG. 20). The Beta diversity plot on the left is annotated by the DMM cluster assignment, with cluster 1 in red, cluster 2 in blue and cluster 3 in green. The plot in the middle uses the same coordinates but is labeled by farm group. TA in blue squares, Farm in green triangles, Nonfarm in orange circles. The plot on the right is annotated by the infant's diet at the time the sample was collected. Exclusively breastfed infants are blue stars, exclusively formula-fed infants in red circles, and those with mixed diet of formula and breastfeeding in yellow diamonds. These bar plots show the distribution of farm groups or diet within each DMM cluster. All but two TA fall into cluster 1, and all exclusively formula-fed fall into cluster 2. Clusters 1 and 3 are each driven primarily by one Bifidobacterium species (Bifidobacterium longum and breve, respectively) while Cluster 2 has more diverse drivers with lower weights. In summary, the high-level structure of the data is driven by diet and farm group.

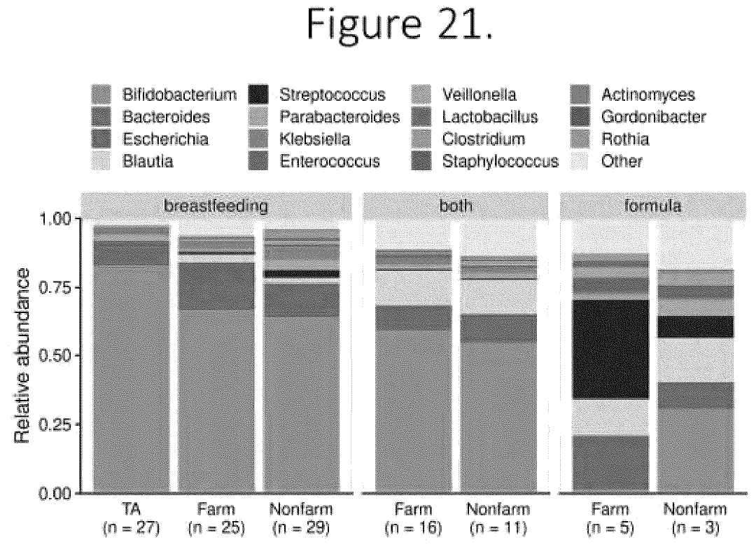

The bars on the left in FIG. 21 show the exclusively breastfeeding infants, in the middle those with mixed diet, and on the right the exclusively formula-fed. The dominant genus across most of these categories is Bifidobacterium, shown in yellow. While all of the breastfed infants had very high Bifidobacterium, the TA infants have relatively higher Bifidobacterium and lower diversity of other microbes. Other patterns are associated with diet. For example, infants with any formula have more Blautia (light blue). The few exclusively formula fed infants had lower abundance of Bifidobacterium and higher abundance of Streptococcus (red).

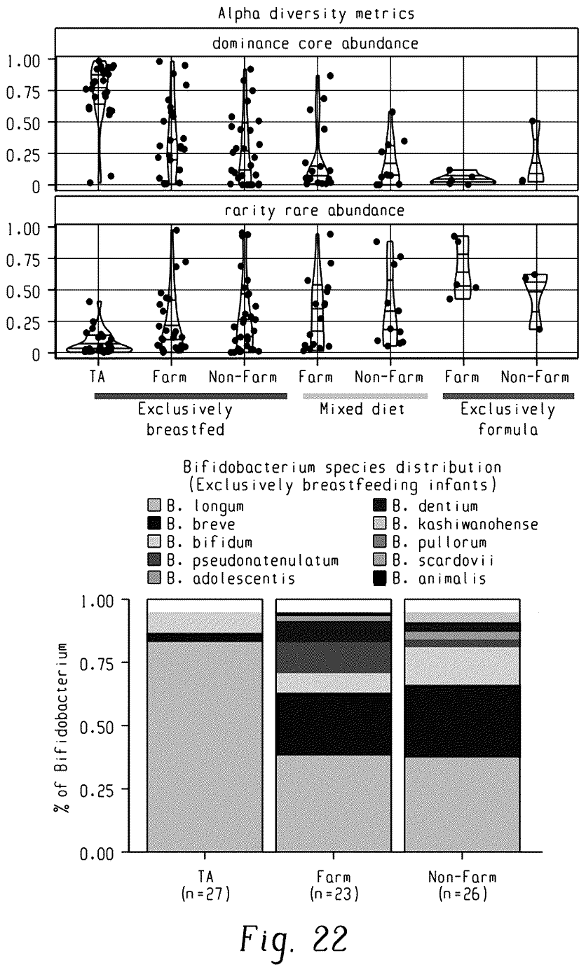

FIG. 22 shows non-TA infants have more diverse microbiomes at species level. The samples were compared based on species-level alpha diversity. The figure provides two example metrics that are significantly associated with diet and with farm group independent of diet. A pattern was observed that the TA infants had lower diversity but higher dominance metrics, which summarize the relative contribution of the most abundant species. The Bifidobacterium species that made up the genus level totals were characterized. The bar plot includes only exclusively breastfeeding infants with any detected Bifidobacterium and shows the average distribution of Bifidobacterium species. The TA infants are predominantly colonized by longum, shown in light blue, while the farm and nonfarm have a greater diversity of species. Thus, the diversity metrics are capturing this predominance of Bifidobacterium longum in the TA samples.

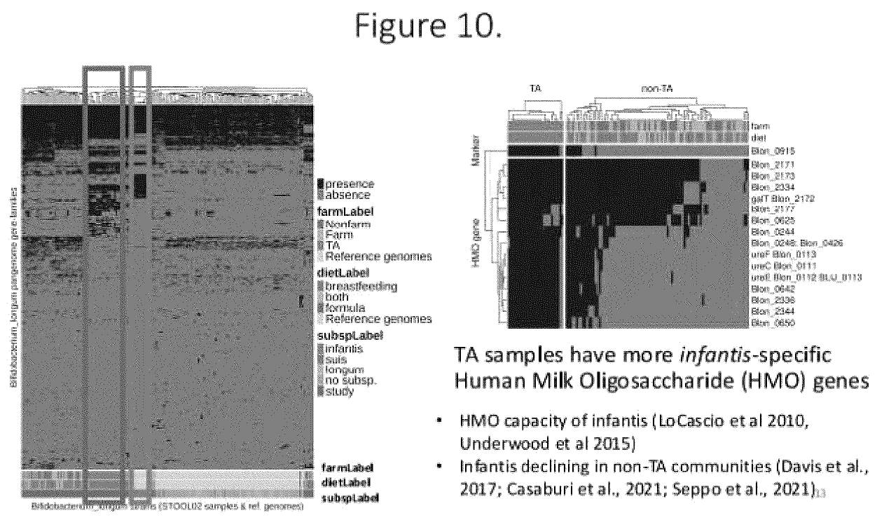

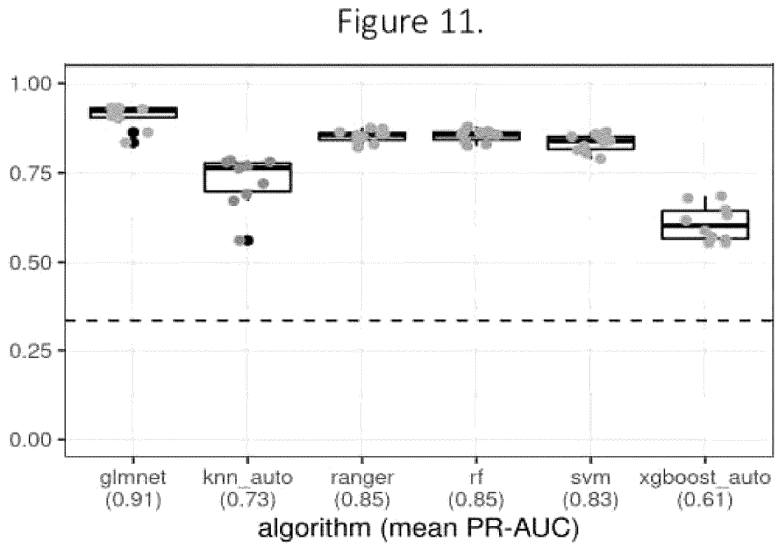

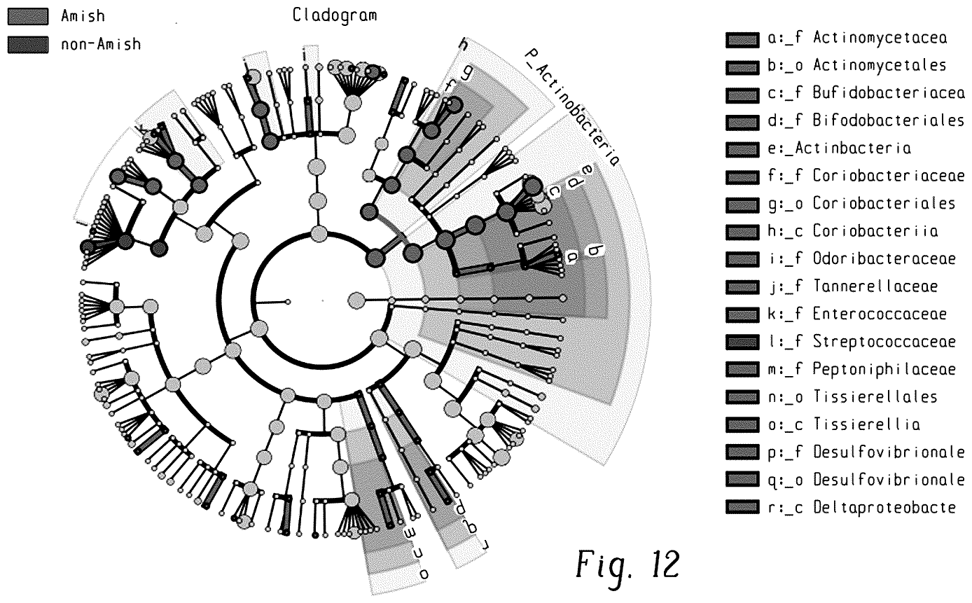

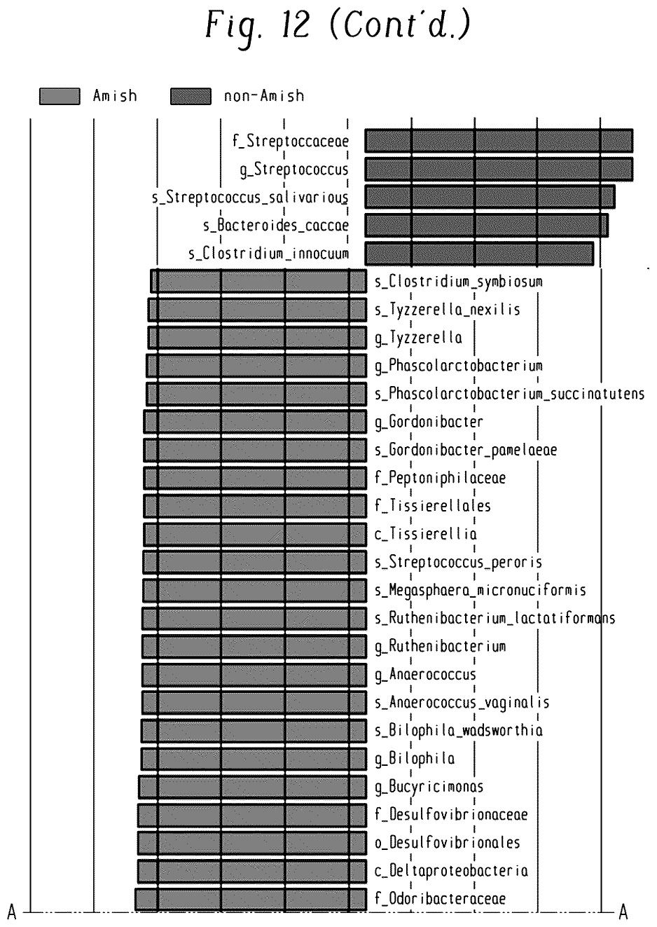

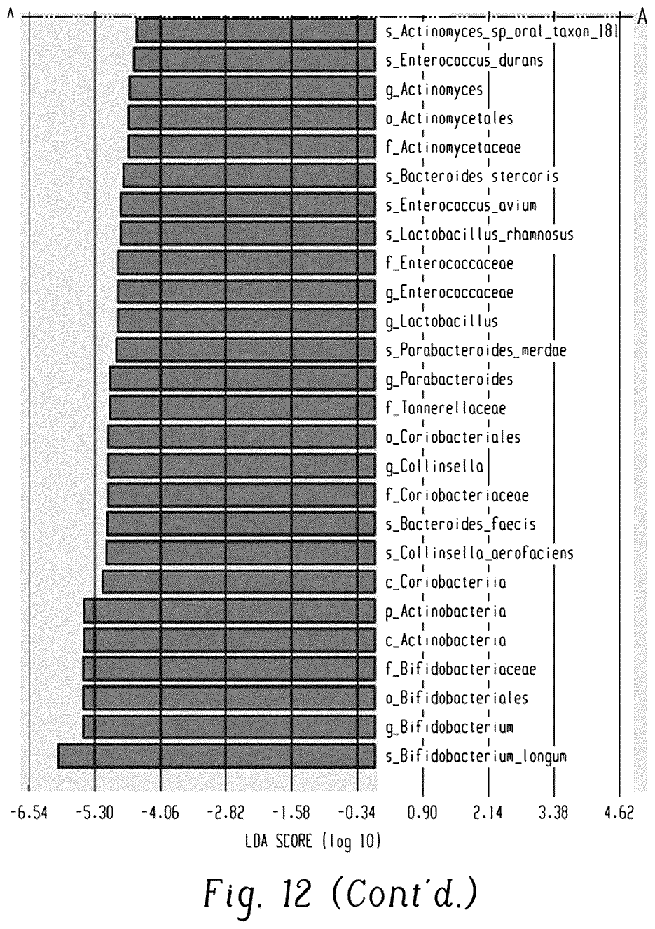

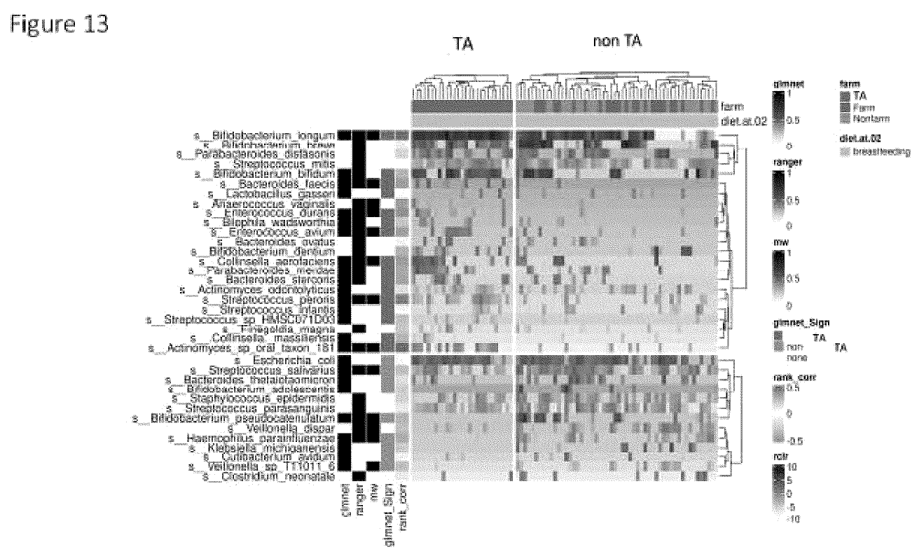

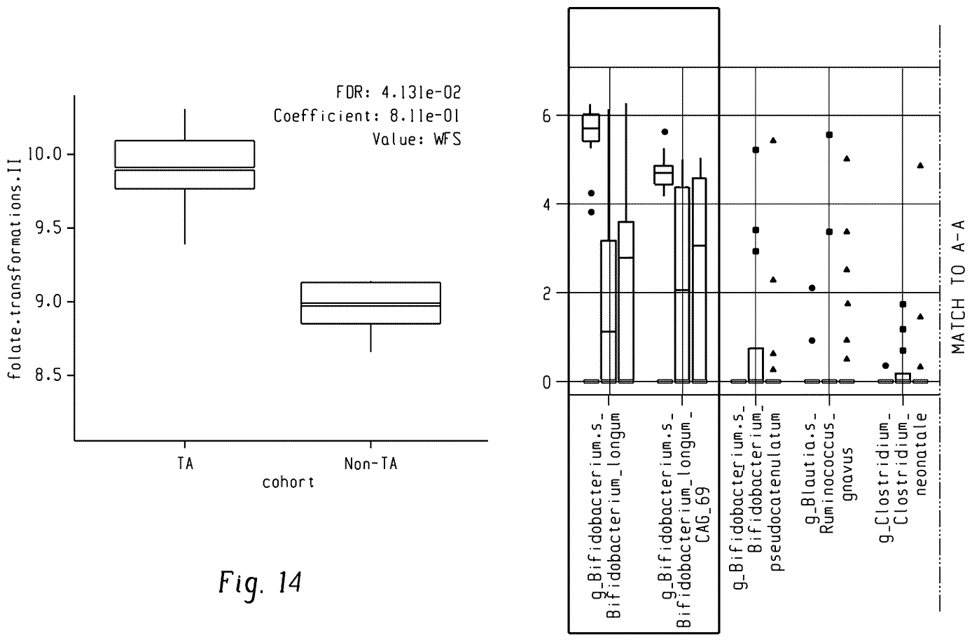

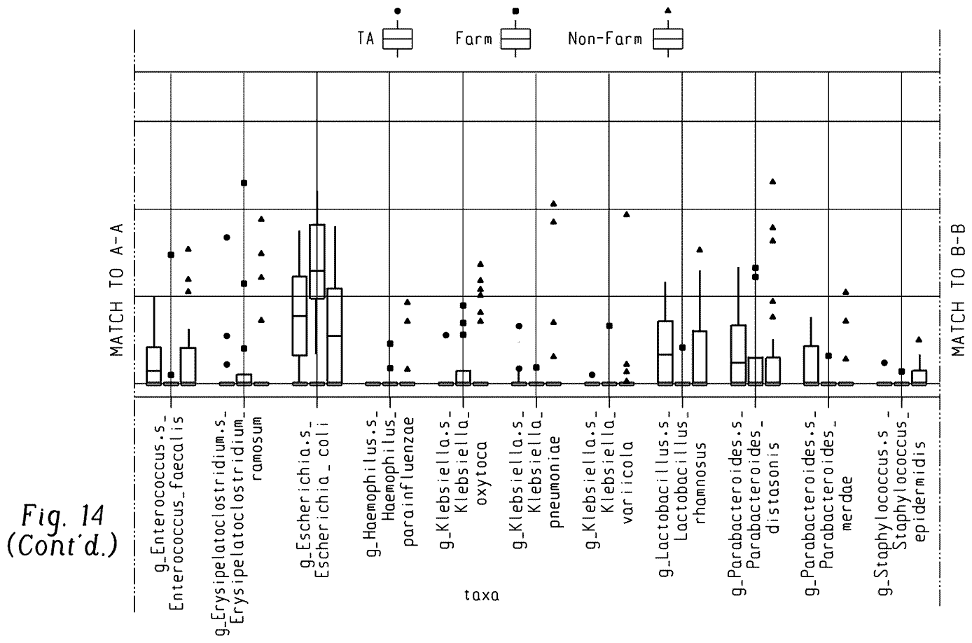

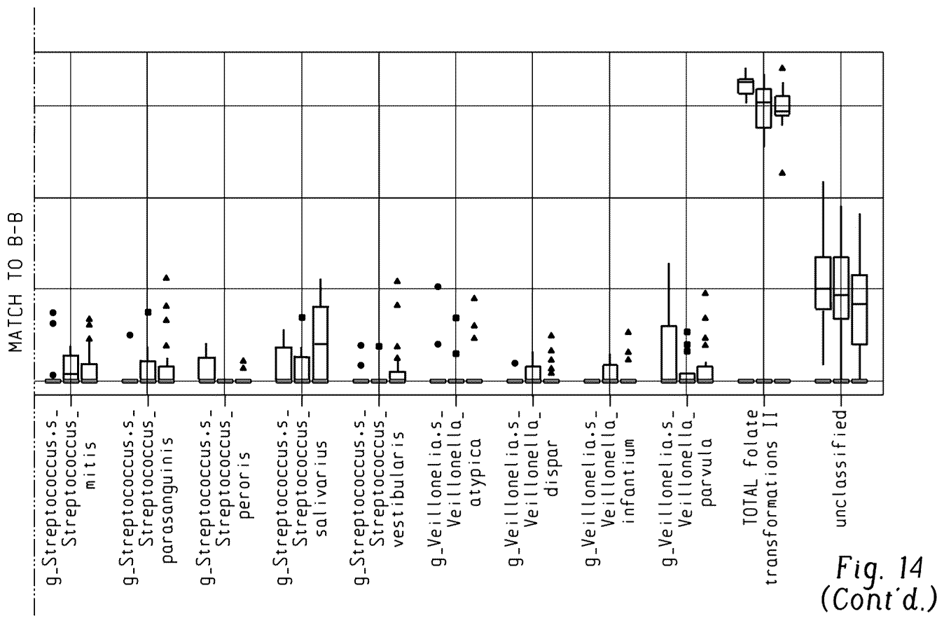

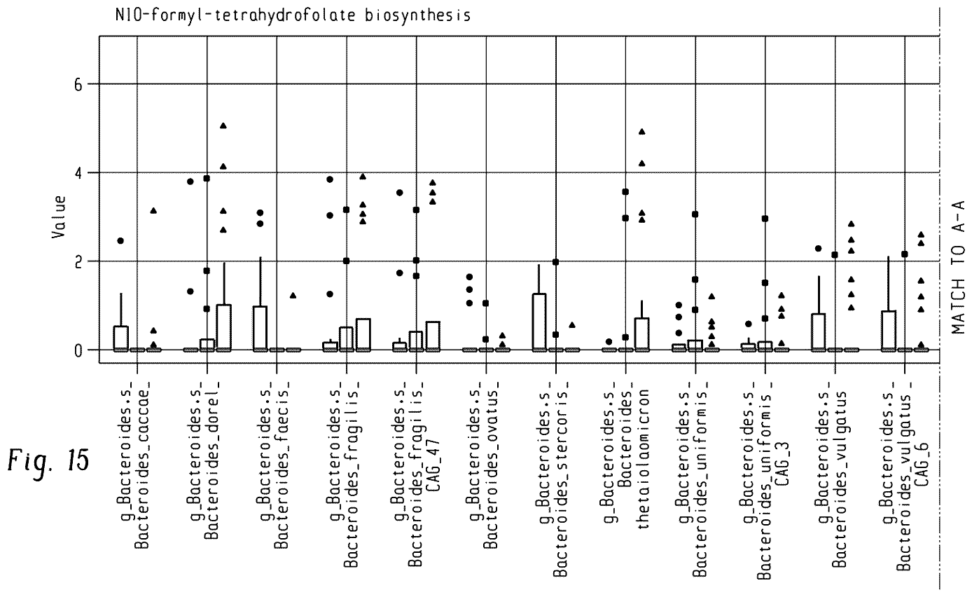

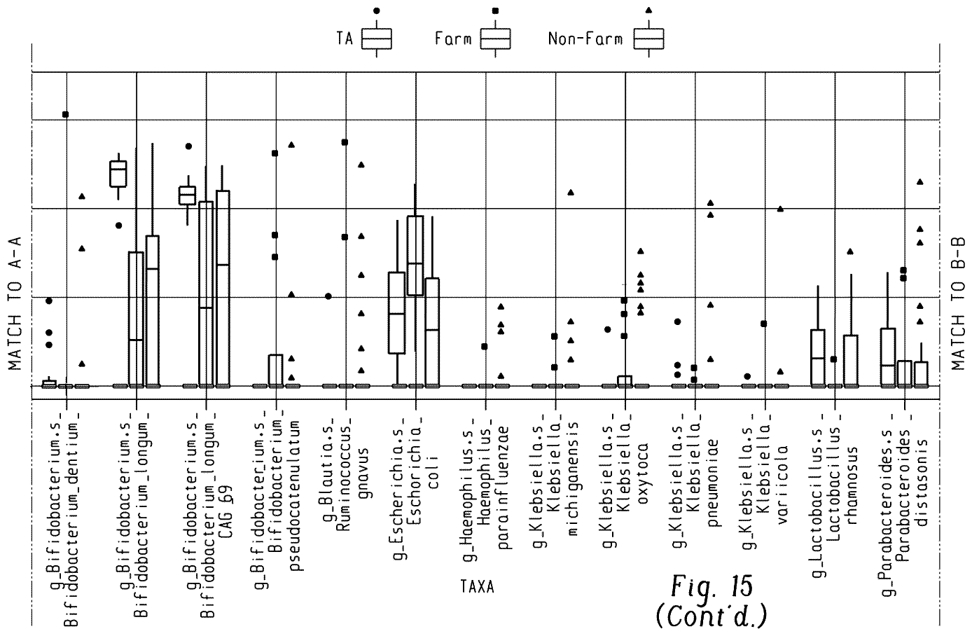

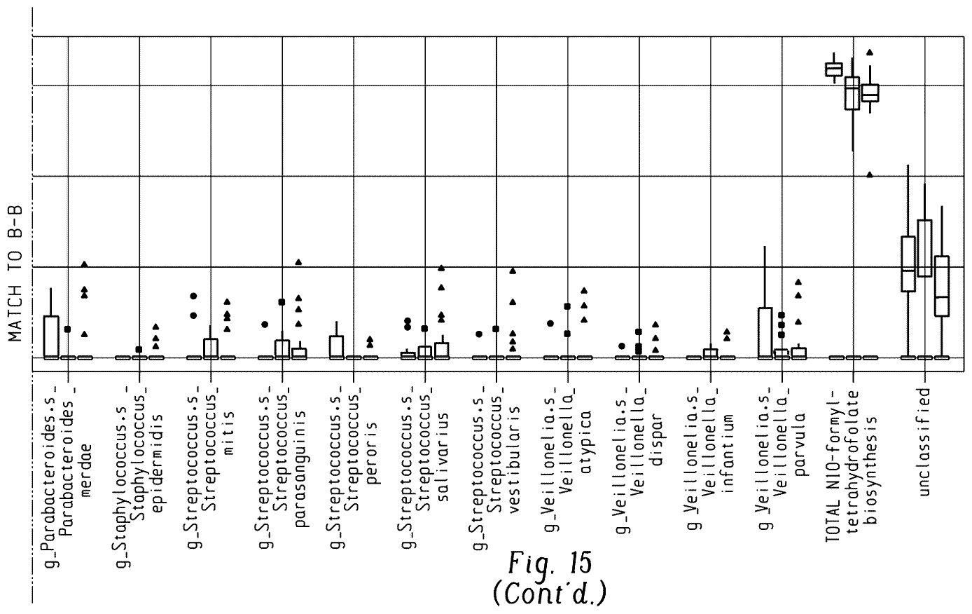

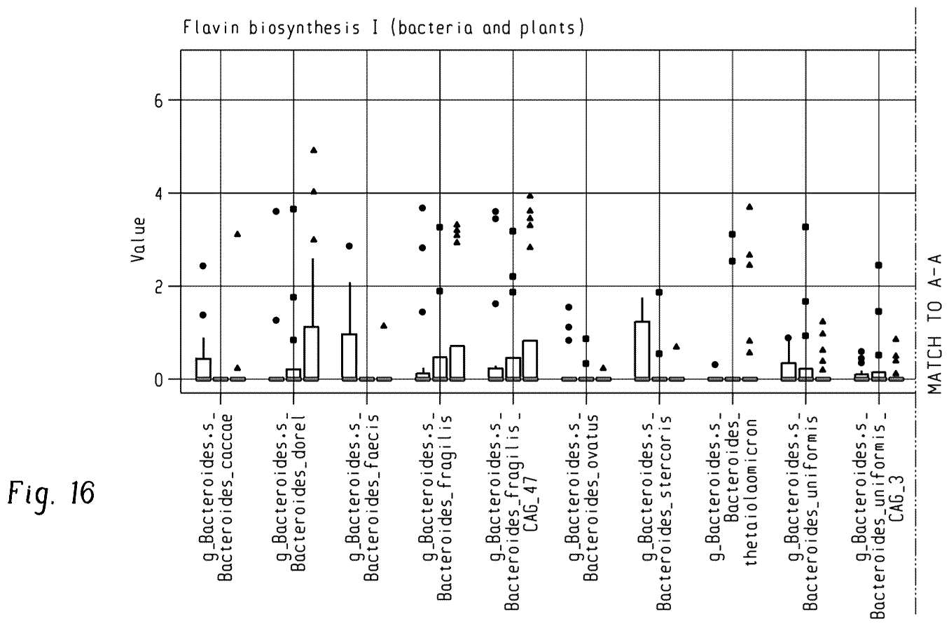

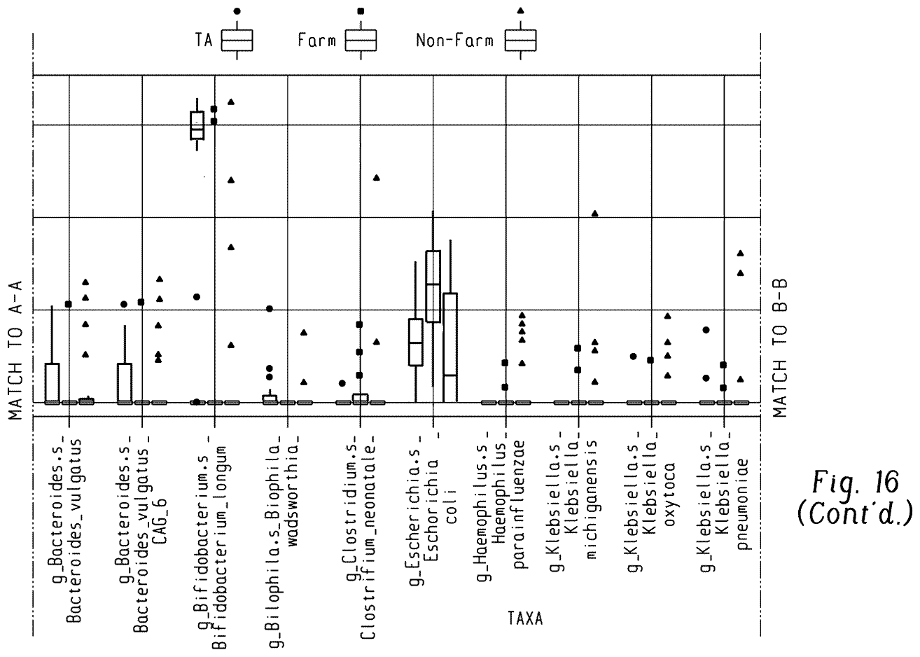

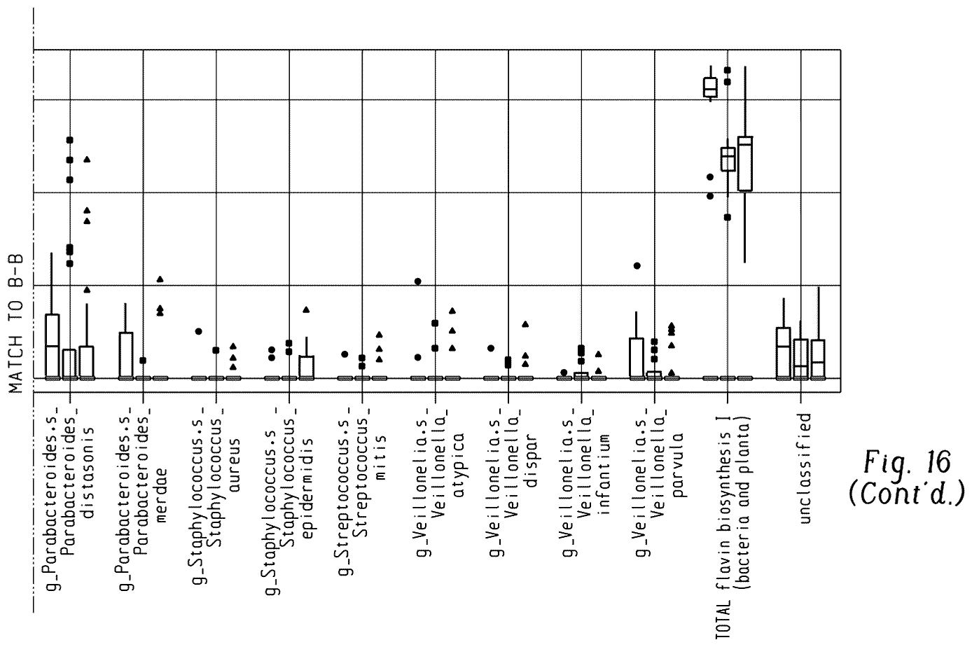

A pangenome analysis was performed (FIG. 10) to survey which Bifidobacterium genes were present in each sample and to compare them to reference genomes. Each row in the heatmap represents a UniRef90 gene family that was found in at least one publicly available reference genome for Bifidobacterium longum. Each column is either a study sample or a reference genome. Red indicates presence and orange indicates absence of the gene. The first bottom annotation indicates reference genomes in light blue, TA by dark blue, farm by green, and nonfarm by orange. A diet annotation with exclusive breastfeeding is also included in blue. For reference genomes, subspecies annotation, if available, is shown in the bottom row annotation. Subspecies infantis are shown in pink, suis in green, and longum in blue. Using hierarchical clustering to compare the gene family representation in our study samples with reference genomes, this heatmap shows that TA samples clustered next to known infantis strains (boxes). However, some differences are observed between the TA study samples compared to infantis references, suggesting the TA samples may have different functional capacity. Finding more similarity between infantis and the TA study samples compared to the non-TA samples is consistent with a body of work that has observed a decline in infantis prevalence in cities and Western lifestyles compared to traditional agrarian communities.

Bifidobacterium infantis has a full complement of genes for metabolizing human milk oligosaccharides and other components of breast milk, whereas other related species have fewer genes, although they can perform cross-feeding. TA samples were confirmed to have greater prevalence of HMO metabolism genes compared to the non-TA. The heatmap shows a subset of HMO genes that are found in the reference files packaged with HUMAnN3. The top half of genes are found broadly in Bifidobacterium longum, while the bottom half are specific to infantis.

Although profiles for all metagenomics samples that were sequenced were computed, to remove the confounding effect of infant diet, the analysis was restricted to TA and exclusively breastfeeding non-TA only. Although HUMANn3 provides community level as well as species-level abundances, significant pathways at the community level were identified. Benjamini-Hochberg was again used to adjust the p-values for community level pathways and called significant those with adjusted p<0.25 (threshold from MaAslin2). After calling significant pathways, the species-level abundances per pathway identified which organisms were involved,

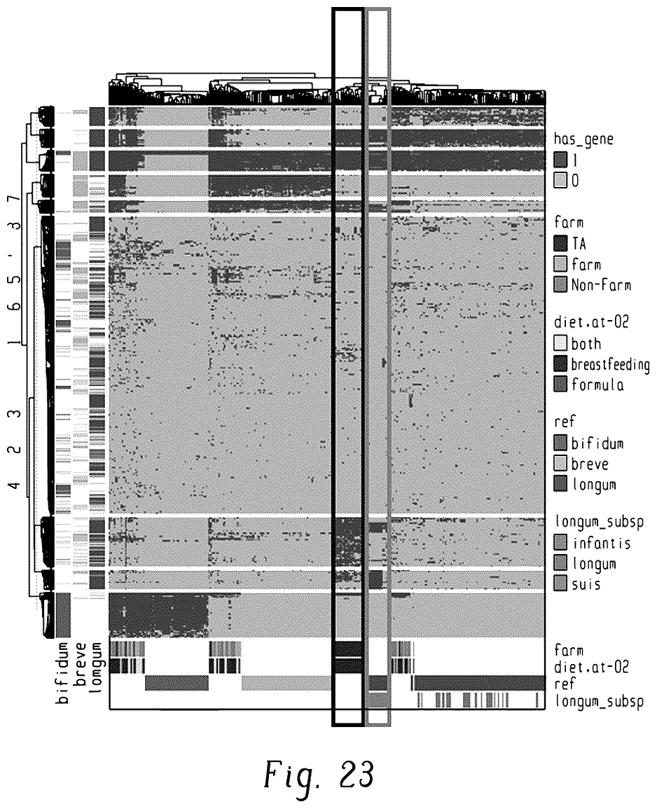

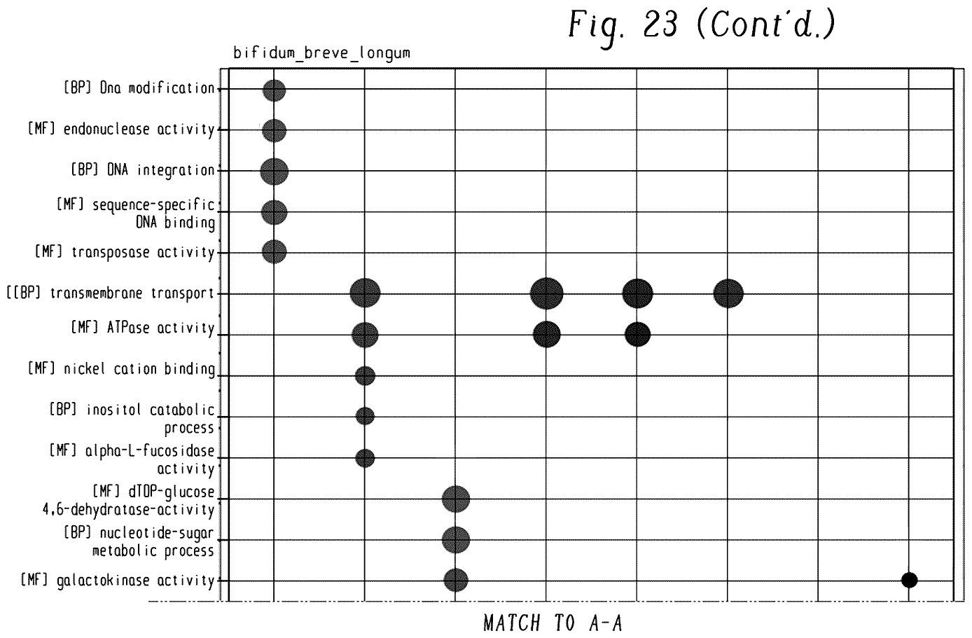

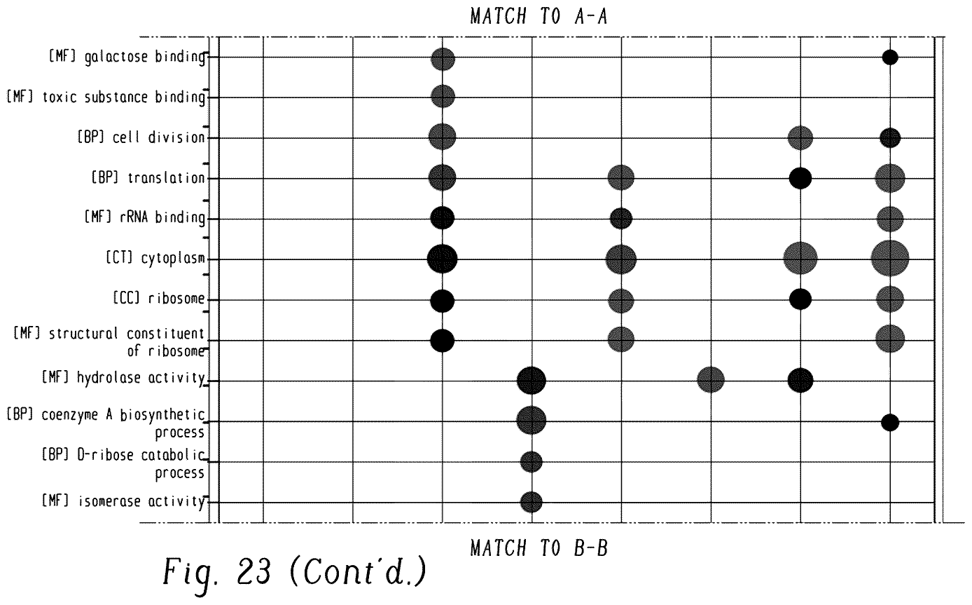

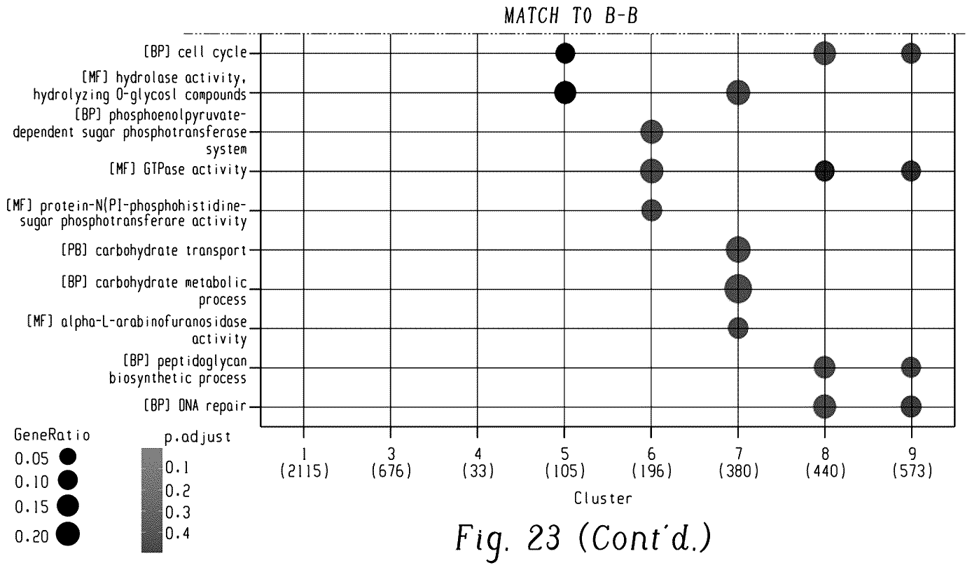

For the data in FIG. 23, “pangenome” files were obtained for B. longum, B. breve, and B. bifidum. These pangenomes do not include all genes found in those organisms, curiously. The three pangenomes were concatenated and for each sample and each reference genome, PanPhlan was used to determine presence/absence of gene in sample. Clustered genes (rows) used k-means, k=8, and hypergeometric tests were run to ask about enrichment of GO terms in clusters (shown in plot on the right). Clusters 2 and 3 are particularly interesting because they have high prevalence in WFS samples and B infantis references.

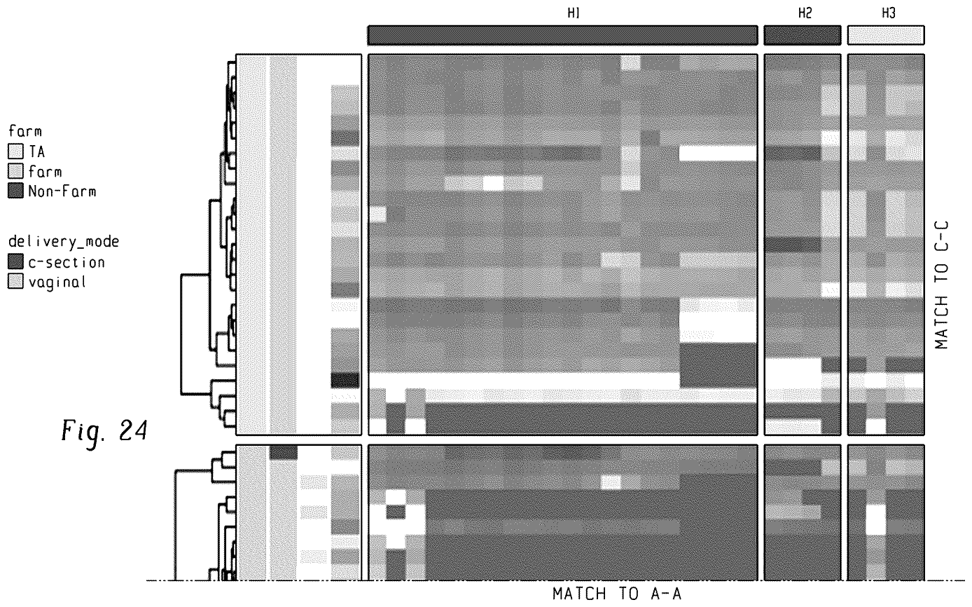

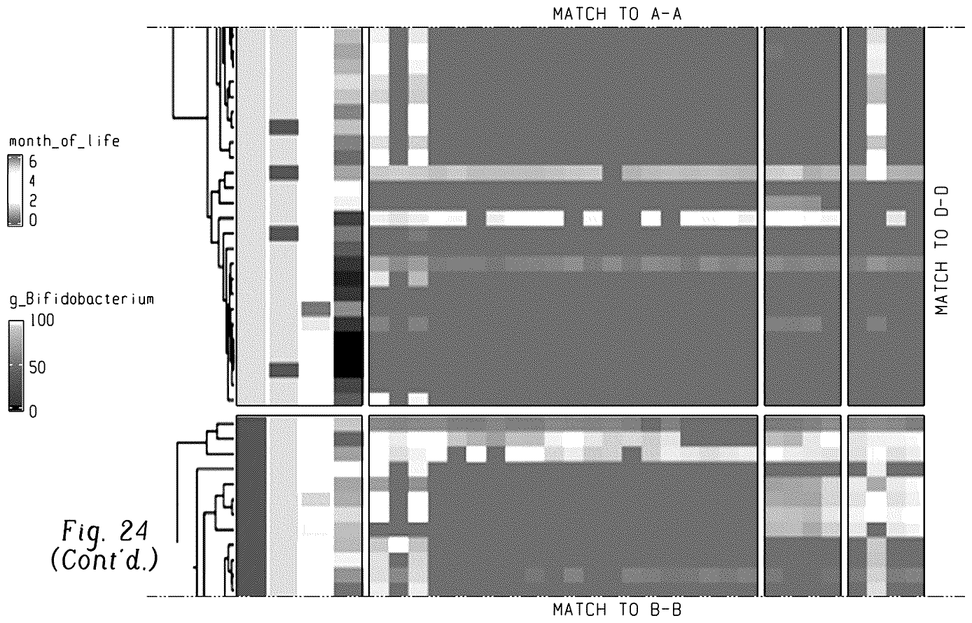

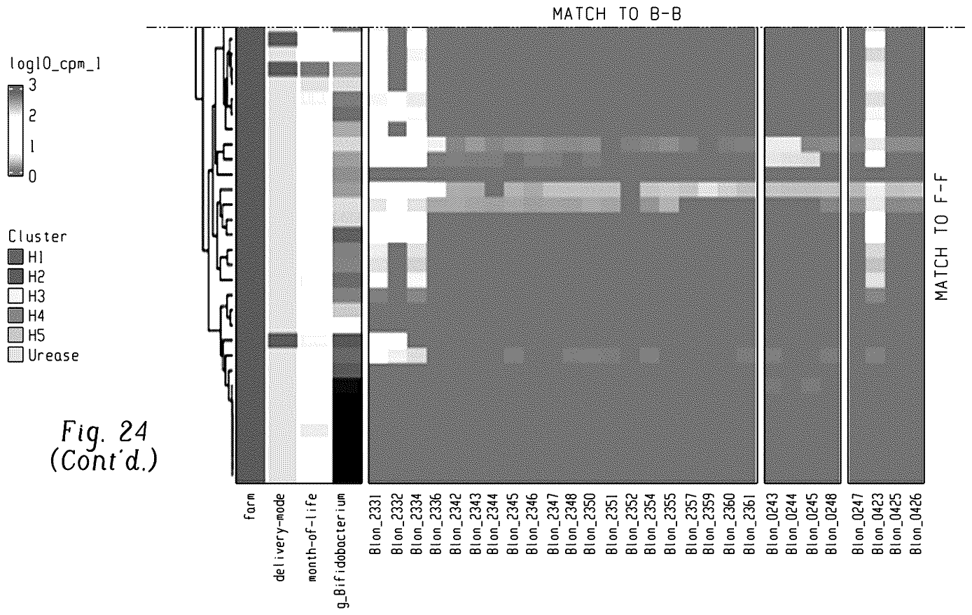

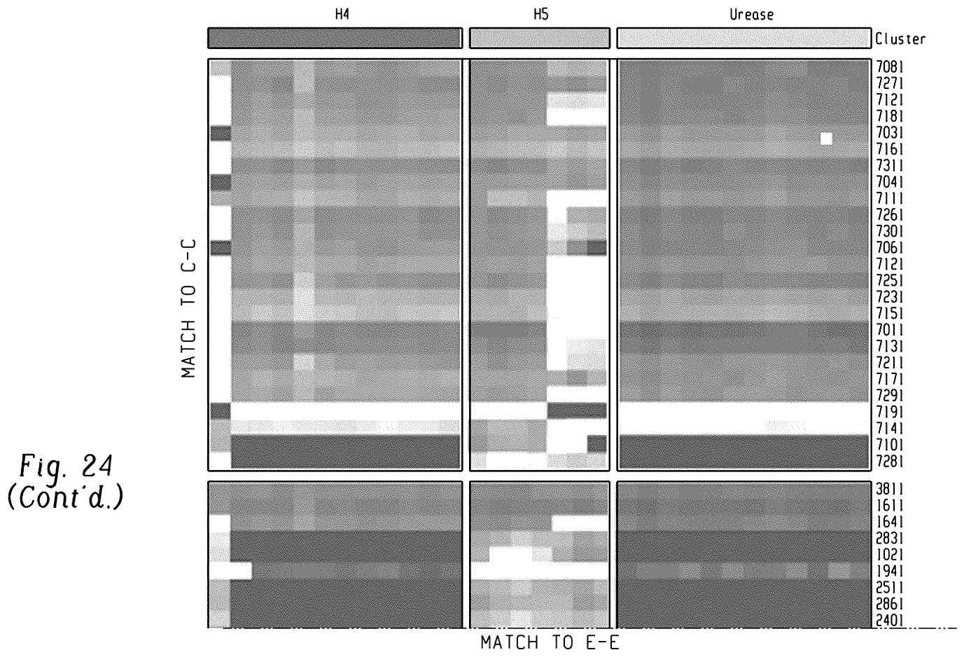

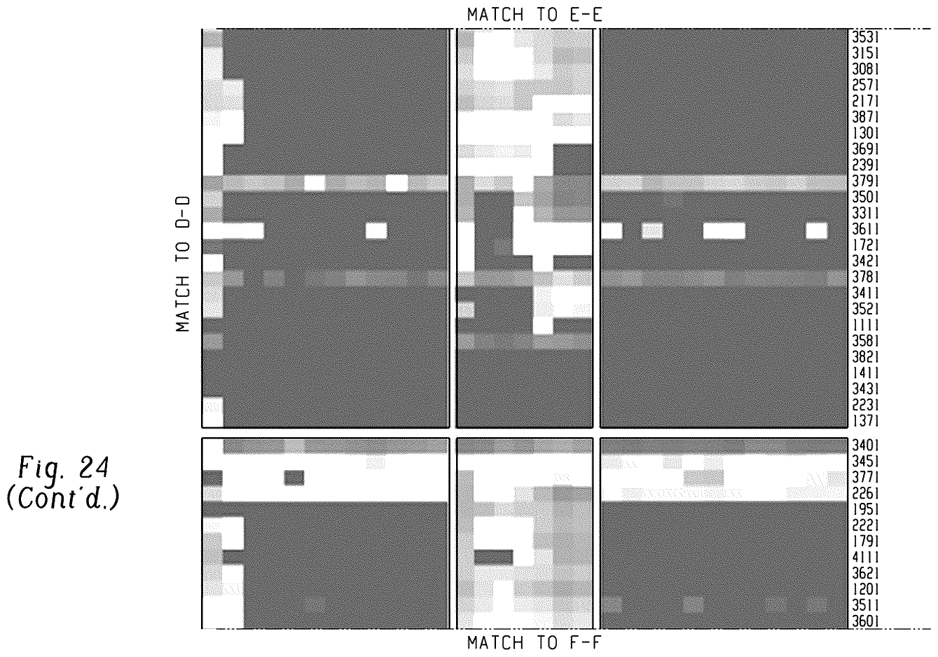

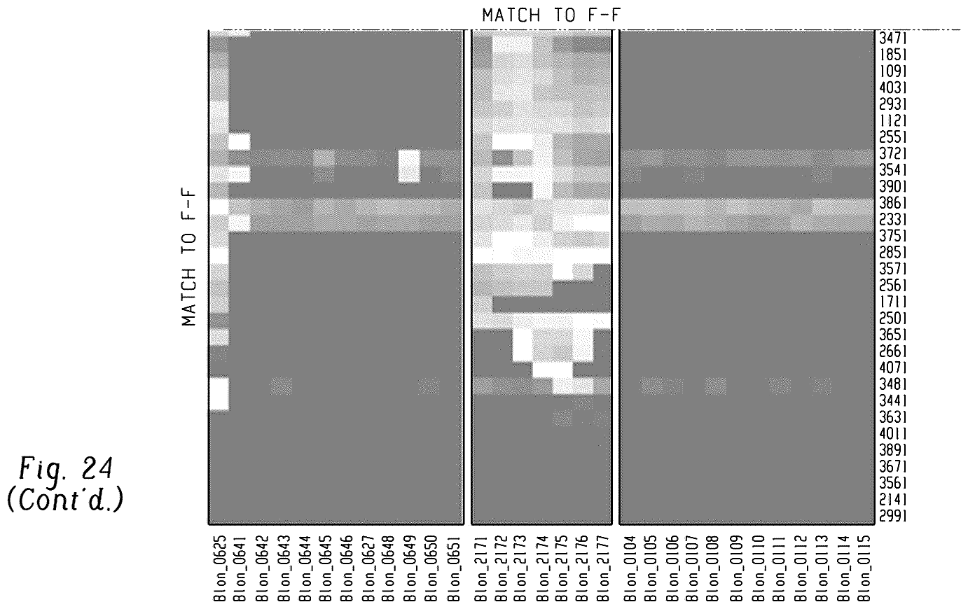

FIG. 24 shows WISC/WFS HMO profiles. Values are log 10(CPM+1). Top annotation is gene cluster, which is based on the organization of the genes on the B. infantis genome. Most of these genes, e.g., most of the genes in clusters H1 (Blon_2331-2361), H2 (Blon_0243-Blon_0248), H3 (Blon_0247, Blon_0244-Blon_0248), H4 (Blon_0625; Blon_0641-Blon_0651), and Urease (Blon_0104-Blon_0115), are highly prevalent among TA samples and not among WISC samples. The H5 cluster of genes is, e.g., Blon_2171-Blon_2177. LoCascio reports that H5 is found commonly in other B. longum strains, so it is not surprising to see that it is prevalent in Farm and Nonfarm as well.

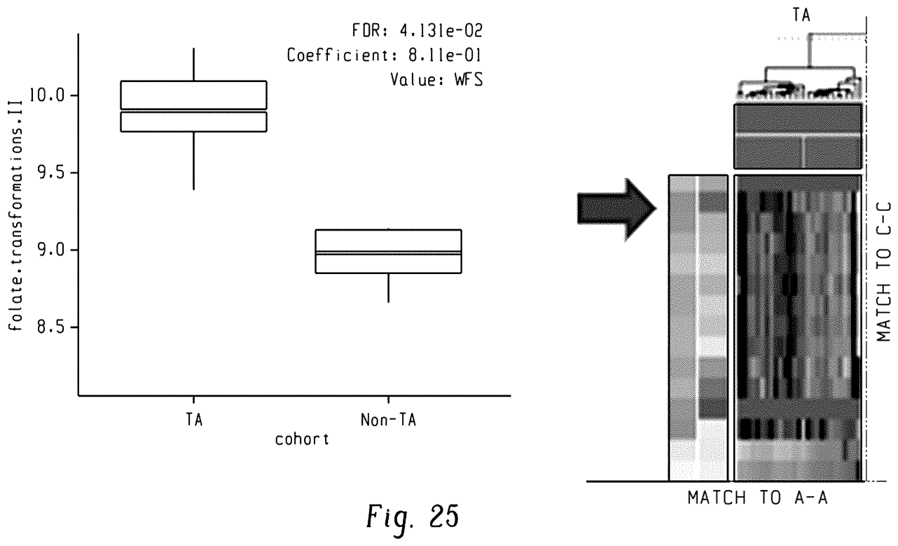

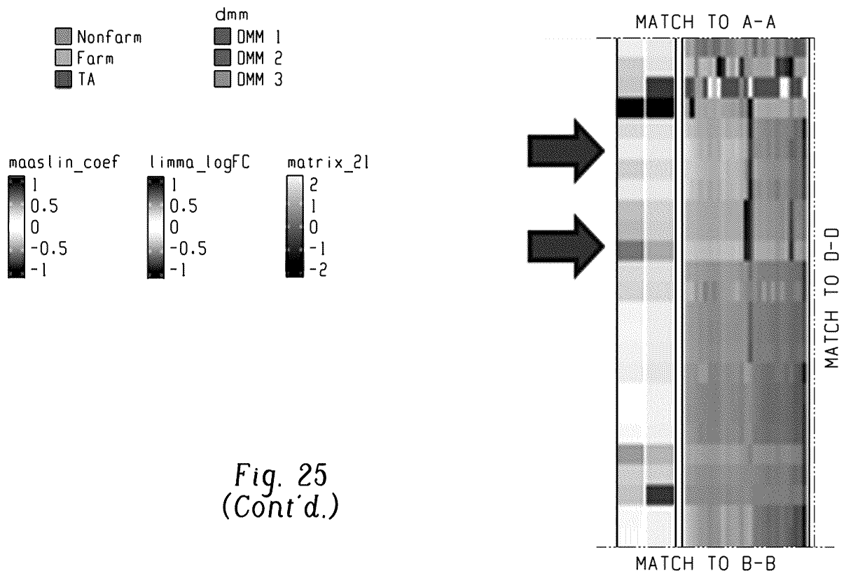

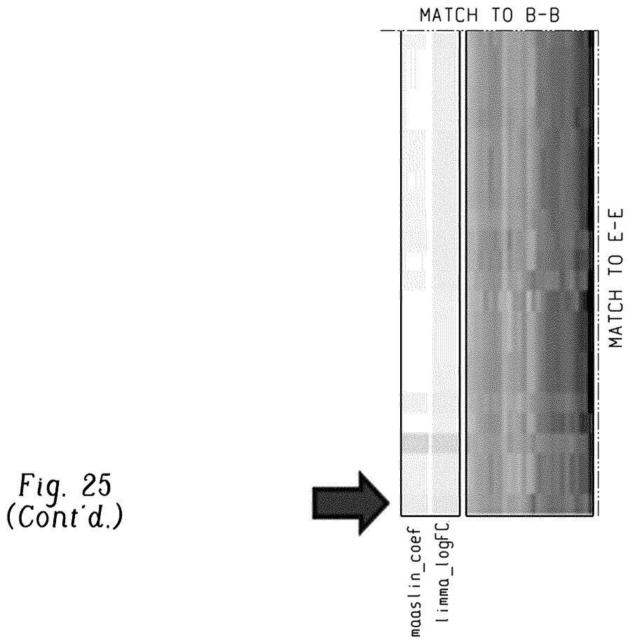

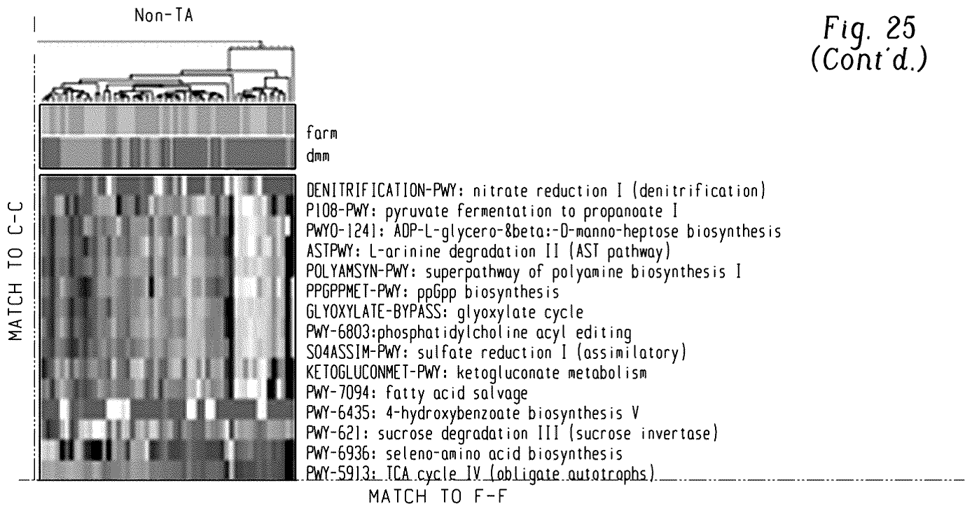

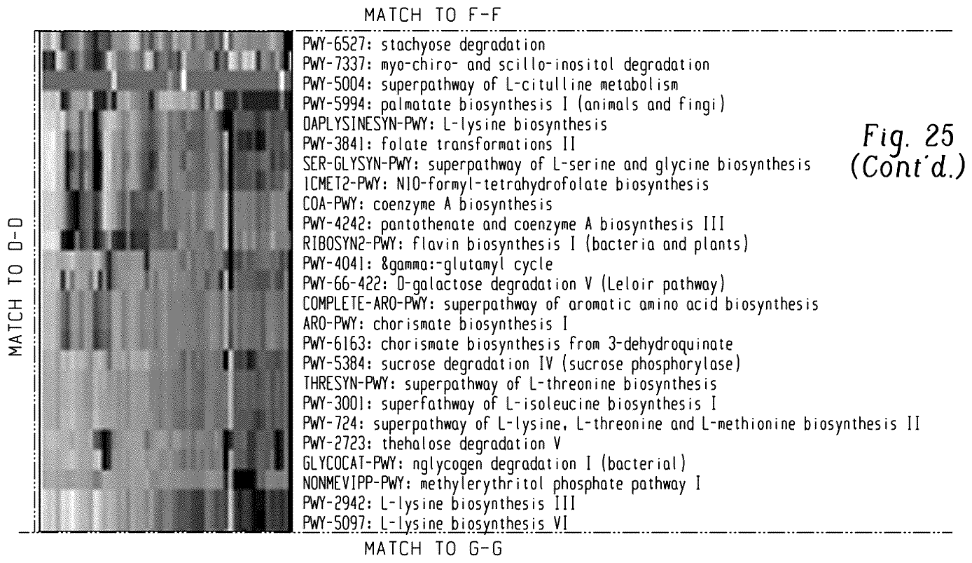

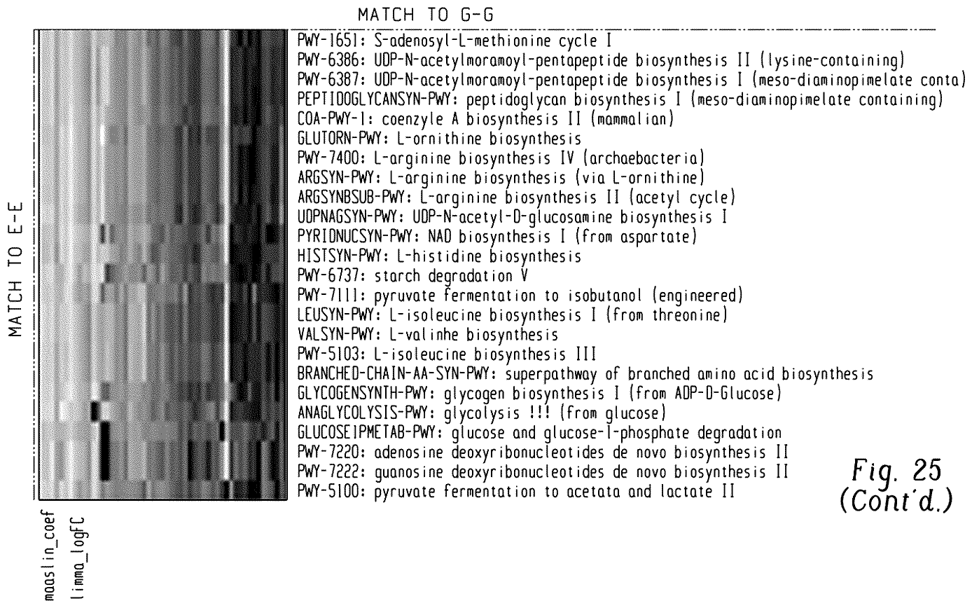

FIG. 25 illustrates differential functional capacities. The analysis was altered by comparing the genetic capacities for metabolic pathways between the groups. The heatmap summarizes the significant pathways, where we took the intersection of hits from two different statistical tests, Maaslin2 and Limma. One of the most significant pathways was folate transformations II, which pertains to B vitamin metabolism. The TA infants have a higher abundance of reads for this pathway than the non-TA infants. Both bacteria and humans need these B vitamins for development. Differences in pathways for short chain fatty acids and amino acid metabolism were also seen.

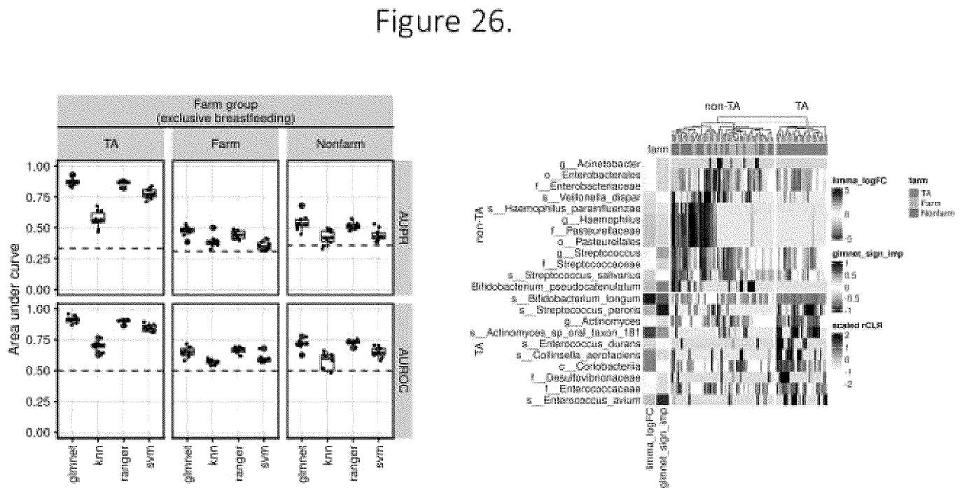

Machine learning models trained on stool metagenomics profiles can distinguish TA from non-TA (FIG. 26). Machine learning models were trained on other organisms or interactions among organisms distinguished the groups and it was observed that it was possible to distinguish the TA samples with high accuracy. ML method performance from 10 repeats of 10-fold CV are shown in the area under the precision-recall curve (PR-AUC) on top and ROC-AUC on the bottom. This is a three-way classification task, and the performance with respect to each target class is shown. For AUPR, the dashed line in each panel is set at the fraction of examples with that class label. For ROC-AUC it is always at 0.5. Elastic networks (glmnet) and random forests (ranger) perform very well on classifying TA samples, and have some more modest signal to distinguish the farm and non-farm groups.

Next, the features used in the elastic network models distinguishing TA from non-TA were inspected. The heatmap shows the top features as well as top differentially abundant microbes. The bottom half is higher in TA and includes Bifidobacterium longum as well as some less abundant distinguishing microbes. The top set of microbes are higher in non-TA samples than TA.

Additional machine learning analysis of metabolites and lipids provided in the Tables below.

Cross-validated machine learning analysis was also used to identify metabolites and lipids associated with TA versus non-TA, considering exclusively breastfeeding infants only. Although the untargeted mass spectrometry experiments identified many features, only features with a confident identification were used for this analysis to improve interpretability of the results. For metabolites, methods performed comparably to metagenomics features. Elastic net (glmnet) achieved average PR-AUC 0.95 and random forest achieved average PR-AUC 0.90 (ROC-AUC 0.95 and 0.90, respectively). Performance using lipid features was slightly lower: elastic net achieved average PR-AUC 0.80 and random forest PR-AUC 0.76 (ROC-AUC 0.82 and 0.78). The union of the top 25 metabolite features prioritized by elastic net and random forest is given in the tables below.

TABLE 12
Stool metabolites for distinguishing TA from non-TA infants.

| Asso- | | | Reten- | Mole- |
|---|---|---|---|---|
| ciated | | | tion | cular |
| group | Identification | HMDB ID | Time | Weight |
| TA higher | 5-Aminovaleric | HMDB0003355 | 12.526 | 117.07934 |
| | acid | | | |
| TA higher | Adenine | HMDB0000034 | 6.364 | 135.05405 |
| TA higher | Cytosine | HMDB0000630 | 8.497 | 111.04364 |
| TA higher | DL-Carnitine | HMDB0000062 | 10.615 | 161.10532 |
| TA higher | L-Citrulline | HMDB0000904 | 13.141 | 175.09567 |
| TA higher | L-Methionine | HMDB0000696 | 9.964 | 149.05106 |
| TA higher | L-Phenylalanine | HMDB0000159 | 9.07 | 165.07874 |
| TA higher | L-Serine | HMDB0000187 | 13.113 | 105.04303 |
| TA higher | L-Tryptophan | HMDB0000929 | 9.941 | 204.08986 |
| TA higher | N-Acetyl- | | 13.797 | 301.04642 |
| | hexosamine_ | | | |
| | RT13.797 | | | |
| TA higher | N-Acetylhistamine | HMDB0013253 | 3.37 | 153.09022 |
| TA higher | N-Acetylornithine | HMDB0003357 | 12.375 | 174.10024 |
| TA higher | Uracil | HMDB0000300 | 4.389 | 112.02633 |
| non-TA | 2-Deoxyuridine | HMDB0000012 | 4.742 | 228.07423 |
| higher | | | | |
| non-TA | 9-HpODE | HMDB0242602 | 1.92 | 312.22994 |
| higher | | | | |
| non-TA | Acamprosate | HMDB0014797 | 8.228 | 181.0402 |
| higher | | | | |
| non-TA | Adenosine | HMDB0000050 | 6.767 | 267.09683 |
| higher | | | | |
| non-TA | Alanylalanine | HMDB0028680 | 11.035 | 160.08452 |
| higher | | | | |
| non-TA | Dihydro- | HMDB0251517 | 2.239 | 301.29814 |
| higher | sphingosine | | | |
| non-TA | Glyceric acid | HMDB0000139; | 12.007 | 106.02564 |
| higher | | HMDB0006372 | | |
| non-TA | Hexose | | 13.41 | 180.06272 |
| higher | | | | |
| non-TA | Imidazolelactic | HMDB0002320 | 11.604 | 156.05283 |
| higher | acid | | | |
| non-TA | L-Proline | HMDB0000162 | 10.222 | 115.06372 |
| higher | | | | |
| non-TA | Lenticin | HMDB0061115 | 7.032 | 246.13695 |
| higher | | | | |
| non-TA | Methyl- | HMDB0059711; | 9.392 | 136.06381 |
| higher | nicotinamide | HMDB0000699; | | |
| | | HMDB0003152; | | |
| | | HMDB0246826 | | |
| non-TA | N-Acetylaspartic | HMDB0000812 | 14.431 | 175.04735 |
| higher | acid | | | |
| non-TA | N-Acetyl- | | 12.28 | 301.04644 |
| higher | hexosamine_ | | | |
| | RT12.28 | | | |
| non-TA | N-alpha-L- | HMDB0004620 | 11.992 | 216.12238 |
| higher | Acetyl-arginine | | | |
| non-TA | Succinic acid | HMDB0000254 | 14.695 | 118.02564 |
| higher | | | | |
| non-TA | Sugar acid | | 13.449 | 194.04204 |
| higher | 6C_RT13.449 | | | |
| non-TA | Sugar alcohol 5C | | 9.897 | 152.06765 |
| higher | | | | |
| non-TA | Sugar alcohol 6C | | 11.161 | 182.07834 |
| higher | | | | |
| non-TA | Triethanolamine | HMDB0032538 | 4.288 | 149.10535 |
| higher | | | | |
| non-TA | Uridine | HMDB0000296 | 7.628 | 244.06919 |
| higher | | | | |

TABLE 13
Stool lipids for distinguishing TA from non-TA infants.

| Associated | | | Retention | | |
|---|---|---|---|---|---|
| group | Identification | Lipid Class | Time | Quant Ion | Polarity |
| TA higher | (2E,4E,14E)-13-Hydroperoxy- | FAA | 4.962 | 394.33173 | + |
| | N-(2-methylpropyl)icosa- | | | | |
| | 2,4,14-trienamide | | | | |
| TA higher | Cer[AP] t40:0 | Cer[AP] | 11.815 | 654.60638 | − |
| TA higher | Cer[AS] d18:2_23:0 | Cer[AS] | 11.9 | 648.59442 | − |
| TA higher | Cer[NS] d18:1_17:0 | Cer[NS] | 10.035 | 534.52539 | + |
| TA higher | Cer[NS] d34:1 (s2lip_121) | Cer[NS] | 8.574 | 520.50848 | + |
| TA higher | Cer[NS] d42:2 (s2lip_276) | Cer[NS] | 11.34 | 630.61792 | + |
| TA higher | Docosahexaenoic acid (DHA) | FA | 8.546 | 329.24762 | + |
| TA higher | PG 22:6_22:6 | PG | 6.9 | 865.50311 | − |
| TA higher | Plasmanyl-PC O-38:1 | Plasmanyl- | 12.024 | 802.6698 | + |
| | (s2lip_303) | PC | | | |
| TA higher | Plasmanyl-PC O-40:4 | Plasmanyl- | 10.811 | 824.65356 | + |
| | | PC | | | |
| TA higher | SP d17:1 | SP | 2.473 | 286.27399 | + |
| TA higher | TG 22:4 22:4 22:4 | TG | 16.527 | 1057.81787 | + |
| | (s2lip_408) | | | | |
| non-TA | Alkanyl-DG O-34:3 (s2lip_229) | Alkanyl-DG | 10.232 | 577.51941 | + |
| higher | | | | | |
| non-TA | Alkenyl-TG P-52:1 | Alkenyl-TG | 18.795 | 862.82202 | + |
| higher | | | | | |
| non-TA | CE 20:3 | CE | 17.265 | 692.63446 | + |
| higher | | | | | |
| non-TA | Cer[AP] t42:1 | Cer[AP] | 12.229 | 680.62048 | − |
| higher | | | | | |
| non-TA | Cer[NS] d18:1_24:0 | Cer[NS] | 14.171 | 708.65216 | − |
| higher | | | | | |
| non-TA | Cer[NS] d18:2_24:0 | Cer[NS] | 13.597 | 706.63623 | − |
| higher | | | | | |
| non-TA | Cer[NS] d36:3 (s2lip_174) | Cer[NS] | 9.316 | 544.50946 | + |
| higher | | | | | |
| non-TA | Cer[NS] d38:0 (s2lip_306) | Cer[NS] | 12.12 | 654.60553 | − |
| higher | | | | | |
| non-TA | Cer[NS] d40:1 (s2lip_310) | Cer[NS] | 12.204 | 620.59949 | − |
| higher | | | | | |
| non-TA | LysoPE 16:0 | LysoPE | 1.668 | 452.27853 | − |
| higher | | | | | |
| non-TA | LysoPE 16:1 | LysoPE | 1.244 | 450.26312 | − |
| higher | | | | | |
| non-TA | LysoPE 17:1 | LysoPE | 1.482 | 464.27853 | − |
| higher | | | | | |
| non-TA | LysoPG 16:0 (s2lip_17) | LysoPG | 1.276 | 483.27298 | − |
| higher | | | | | |
| non-TA | PC 33:1 (s2lip_179) | PC | 9.364 | 746.57843 | + |
| higher | | | | | |
| non-TA | PC 35:2 (s2lip_188) | PC | 9.518 | 772.59277 | + |
| higher | | | | | |
| non-TA | PE 16:0_17:1 | PE | 9.51 | 702.50928 | − |
| higher | | | | | |
| non-TA | PE 16:0_18:1 (s2lip_204) | PE | 9.794 | 718.53839 | + |
| higher | | | | | |
| non-TA | PE 16:0_18:2 | PE | 9.089 | 714.50909 | − |
| higher | | | | | |
| non-TA | PE 28:0 | PE | 8.005 | 636.45966 | + |
| higher | | | | | |
| non-TA | PE 29:0 | PE | 8.277 | 648.46161 | − |
| higher | | | | | |
| non-TA | PE 30:1 | PE | 8.116 | 660.46185 | − |
| higher | | | | | |
| non-TA | PE 31:0 | PE | 9.119 | 678.50629 | + |
| higher | | | | | |
| non-TA | PE 32:1 | PE | 8.917 | 688.49292 | − |
| higher | | | | | |
| non-TA | PE 34:2 (s2lip_148) | PE | 8.949 | 716.52179 | + |
| higher | | | | | |
| non-TA | PE 34:2 (s2lip_171) | PE | 9.256 | 716.52295 | + |
| higher | | | | | |
| non-TA | PE 36:5 | PE | 9.068 | 738.50354 | + |
| higher | | | | | |
| non-TA | PG 18:1_18:1 | PG | 8.481 | 773.5354 | − |
| higher | | | | | |
| non-TA | PI 16:0_18:1 | PI | 8.256 | 835.53711 | − |
| higher | | | | | |

TABLE 14
Stool lipids from untargeted mass spectrometry analysis that are correlated to TA-associated
microbial pathways and may be indicative of decreased risk of allergic disease.

| Unique Lipid | Retention | | | |
|---|---|---|---|---|
| ID | time | Quant ion | Polarity | Identification |
| s2lip_1856 | 7.031 | 547.40125 | − | Unknown_mz547.40125_−_RT7.031 |
| s2lip_1571 | 5.95 | 581.36212 | − | Unknown_mz581.36212_−_RT5.95 |
| s2lip_1855 | 7.031 | 583.37781 | − | Unknown_mz583.37781_−_RT7.031 |
| s2lip_1860 | 7.033 | 593.40692 | − | Unknown_mz593.40692_−_RT7.033 |
| s2lip_1570 | 5.95 | 605.40637 | − | Unknown_mz605.40637_−_RT5.95 |
| s2lip_1858 | 7.033 | 607.42255 | − | Unknown_mz607.42255_−_RT7.033 |
| s2lip_2013 | 7.277 | 609.43762 | − | Unknown_mz609.43762_−_RT7.277 |
| s2lip_6153 | 12.462 | 793.57672 | − | Unknown_mz793.57672_−_RT12.462 |
| s2lip_1868 | 7.035 | 803.6405 | − | Unknown_mz803.6405_−_RT7.035 |
| s2lip_1569 | 5.949 | 815.64166 | − | Unknown_mz815.64166_−_RT5.949 |
| s2lip_6154 | 12.463 | 817.62134 | − | Unknown_mz817.62134_−_RT12.463 |
| s2lip_1853 | 7.03 | 831.67328 | − | Unknown_mz831.67328_−_RT7.03 |
| s2lip_6177 | 12.503 | 843.63611 | − | Unknown_mz843.63611_−_RT12.503 |
| s2lip_6637 | 13.432 | 845.65088 | − | Unknown_mz845.65088_−_RT13.432 |
| s2lip_1842 | 7.022 | 845.68896 | − | Unknown_mz845.68896_−_RT7.022 |
| s2lip_6643 | 13.441 | 847.62115 | − | Unknown_mz847.62115_−_RT13.441 |
| s2lip_6642 | 13.441 | 857.65302 | − | Unknown_mz857.65302_−_RT13.441 |
| s2lip_594 | 0.754 | 861.60938 | − | Unknown_mz861.60938_−_RT0.754 |
| s2lip_6997 | 14.137 | 861.63867 | − | Unknown_mz861.63867_−_RT14.137 |
| s2lip_6213 | 12.573 | 869.65259 | − | Unknown_mz869.65259_−_RT12.573 |
| s2lip_6647 | 13.443 | 871.6684 | − | Unknown_mz871.6684_−_RT13.443 |
| s2lip_7102 | 14.399 | 873.68286 | − | Unknown_mz873.68286_−_RT14.399 |
| s2lip_6999 | 14.138 | 885.68402 | − | Unknown_mz885.68402_−_RT14.138 |
| s2lip_7668 | 15.847 | 1053.81946 | − | Unknown_mz1053.81946_−_RT15.847 |
| s2lip_3638 | 9.164 | 1079.67383 | − | Unknown_mz1079.67383_−_RT9.164 |
| s2lip_5721 | 11.725 | 1119.77075 | − | Unknown_mz1119.77075_−_RT11.725 |
| s2lip_1578 | 5.957 | 367.33621 | + | Unknown_mz367.33621_+_RT5.957 |
| s2lip_1851 | 7.029 | 369.35153 | + | Unknown_mz369.35153_+_RT7.029 |
| s2lip_6650 | 13.444 | 409.29474 | + | Unknown_mz409.29474_+_RT13.444 |
| s2lip_6651 | 13.446 | 427.30508 | + | Unknown_mz427.30508_+_RT13.446 |
| s2lip_1865 | 7.034 | 566.4422 | + | Unknown_mz566.4422_+_RT7.034 |
| s2lip_1573 | 5.954 | 569.38165 | + | Unknown_mz569.38165_+_RT5.954 |
| s2lip_1852 | 7.03 | 571.39697 | + | Unknown_mz571.39697_+_RT7.03 |
| s2lip_1575 | 5.955 | 585.35571 | + | Unknown_mz585.35571_+_RT5.955 |
| s2lip_1849 | 7.028 | 587.37134 | + | Unknown_mz587.37134_+_RT7.028 |
| s2lip_1579 | 5.957 | 610.40771 | + | Unknown_mz610.40771_+_RT5.957 |
| s2lip_1861 | 7.033 | 612.42352 | + | Unknown_mz612.42352_+_RT7.033 |
| s2lip_6152 | 12.462 | 781.59595 | + | Unknown_mz781.59595_+_RT12.462 |
| s2lip_6655 | 13.455 | 809.62524 | + | Unknown_mz809.62524_+_RT13.455 |
| s2lip_6225 | 12.598 | 828.67358 | + | Unknown_mz828.67358_+_RT12.598 |
| s2lip_6639 | 13.436 | 830.68719 | + | Unknown_mz830.68719_+_RT13.436 |
| s2lip_6221 | 12.586 | 833.62671 | + | Unknown_mz833.62671_+_RT12.586 |
| s2lip_6644 | 13.442 | 835.64264 | + | Unknown_mz835.64264_+_RT 13.442 |
| s2lip_7004 | 14.143 | 849.65796 | + | Unknown_mz849.65796_+_RT14.143 |
| s2lip_6649 | 13.444 | 851.61694 | + | Unknown_mz851.61694_+_RT13.444 |
| s2lip_3561 | 9.112 | 866.66913 | + | Unknown_mz866.66913_+_RT9.112 |
| s2lip_6646 | 13.442 | 911.62836 | + | Unknown_mz911.62836_+_RT13.442 |
| s2lip_6648 | 13.443 | 917.64435 | + | Unknown_mz917.64435_+_RT13.443 |
| s2lip_7667 | 15.847 | 1012.83838 | + | Unknown_mz1012.83838_+_RT15.847 |
| s2lip_7669 | 15.847 | 1017.79352 | + | Unknown_mz1017.79352_+_RT15.847 |
| s2lip_7662 | 15.823 | 1038.85388 | + | Unknown_mz1038.85388_+_RT15.823 |
| s2lip_3637 | 9.164 | 1098.71521 | + | Unknown_mz1098.71521_+_RT9.164 |
| s2lip_3647 | 9.168 | 1103.67017 | + | Unknown_mz1103.67017_+_RT9.168 |
| s2lip_1577 | 5.957 | 1115.7738 | + | Unknown_mz1115.7738_+_RT5.957 |
| s2lip_1850 | 7.029 | 1119.80518 | + | Unknown_mz1119.80518_+_RT7.029 |
| s2lip_1863 | 7.034 | 1121.81189 | + | Unknown_mz1121.81189_+_RT7.034 |

TABLE 15
Stool metabolites from untargeted mass spectrometry analysis that are correlated to
TA-associated microbial pathways and may be indicative of decreased risk of allergic
disease. HMDB ID: Entry for metabolite in Human Metabolome Database (hmdb.ca).

| Unique | | | | |
|---|---|---|---|---|
| metabolite | Retention | Molecular | | |
| ID | time | weight | HMDB ID | Identification |
| s2met_893 | 14.064 | 89.04671 | | unknown_mass89.04671_RT14.064 |
| s2met_872 | 13.804 | 101.04815 | | unknown_mass101.04815_RT13.804 |
| s2met_54 | 8.497 | 111.04364 | HMDB0000630 | Cytosine |
| s2met_951 | 15.055 | 130.02569 | | unknown_mass130.02569_RT15.055 |
| s2met_593 | 9.987 | 134.05697 | | unknown_mass134.05697_RT9.987 |
| s2met_34 | 6.364 | 135.05405 | HMDB0000034 | Adenine |
| s2met_440 | 6.33 | 136.07265 | | unknown_mass136.07265_RT6.33 |
| s2met_906 | 14.206 | 145.07306 | | unknown_mass145.07306_RT14.206 |
| s2met_619 | 10.971 | 146.05709 | | unknown_mass146.05709_RT10.971 |
| s2met_920 | 14.477 | 148.03633 | | unknown_mass148.03633_RT14.477 |
| s2met_952 | 15.055 | 148.03633 | | unknown_mass148.03633_RT15.055 |
| s2met_745 | 12.606 | 150.05199 | | unknown_mass150.05199_RT12.606 |
| s2met_706 | 12.094 | 163.08413 | | unknown_mass163.08413_RT12.094 |
| s2met_623 | 10.987 | 164.0677 | | unknown_mass164.0677_RT10.987 |
| s2met_663 | 11.635 | 164.0677 | | unknown_mass164.0677_RT11.635 |
| s2met_899 | 14.135 | 171.05244 | | unknown_mass171.05244_RT14.135 |
| s2met_502 | 7.977 | 173.06821 | | unknown_mass173.06821_RT7.977 |
| s2met_436 | 6.251 | 176.06775 | | unknown_mass176.06775_RT6.251 |
| s2met_67 | 10.097 | 182.0572 | HMDB0000755 | 4-Hydroxyphenyllactic acid |
| s2met_618 | 10.952 | 188.11623 | | unknown_mass188.11623_RT10.952 |
| s2met_95 | 14.201 | 189.0631 | HMDB0001138 | N-Acetyl-DL-glutamic acid |
| s2met_438 | 6.323 | 196.09406 | | unknown_mass196.09406_RT6.323 |
| s2met_637 | 11.187 | 204.06115 | | unknown_mass204.06115_RT11.187 |
| s2met_909 | 14.271 | 205.05806 | | unknown_mass205.05806_RT14.271 |
| s2met_59 | 9.004 | 205.0733 | HMDB0000671 | Indole-3-lactic acid |
| s2met_905 | 14.19 | 211.04578 | | unknown_mass211.04578_RT14.19 |
| s2met_626 | 11.006 | 217.09509 | | unknown_mass217.09509_RT11.006 |
| s2met_845 | 13.525 | 218.09034 | | unknown_mass218.09034_RT13.525 |
| s2met_896 | 14.098 | 219.07376 | | unknown_mass219.07376_RT14.098 |
| s2met_907 | 14.239 | 227.01973 | | unknown_mass227.01973_RT14.239 |
| s2met_860 | 13.762 | 248.1008 | | unknown_mass248.1008_RT13.762 |
| s2met_947 | 14.903 | 259.03589 | | unknown_mass259.03589_RT14.903 |
| s2met_886 | 13.911 | 268.07928 | | unknown_mass268.07928_RT13.911 |
| s2met_778 | 12.897 | 277.0618 | | unknown_mass277.0618_RT12.897 |
| s2met_82 | 12.229 | 278.09357 | HMDB0034367 | gamma-Glutamylmethionine |
| s2met_953 | 15.082 | 283.96981 | | unknown_mass283.96981_RT15.082 |
| s2met_813 | 13.305 | 287.11181 | | unknown_mass287.11181_RT13.305 |
| s2met_839 | 13.424 | 294.08869 | | unknown_mass294.08869_RT13.424 |
| s2met_883 | 13.869 | 309.10591 | | unknown_mass309.10591_RT13.869 |
| s2met_790 | 13.093 | 312.04481 | | unknown_mass312.04481_RT13.093 |
| s2met_624 | 10.997 | 328.13667 | | unknown_mass328.13667_RT10.997 |
| s2met_857 | 13.696 | 331.18566 | | unknown_mass331.18566_RT13.696 |
| s2met_850 | 13.588 | 335.1327 | | unknown_mass335.1327_RT13.588 |
| s2met_612 | 10.881 | 350.11846 | | unknown_mass350.11846_RT10.881 |
| s2met_541 | 8.782 | 393.12686 | | unknown_mass393.12686_RT8.782 |
| s2met_92 | 13.605 | 398.13746 | HMDB0001185 | S-Adenosylmethionine (SAM-e) |
| s2met_973 | 15.541 | 422.07266 | | unknown_mass422.07266_RT15.541 |

In the comparative genomics analysis by LoCascio et al, specific gene clusters for HMO metabolism were found in infantis but not in longum subspecies. Infantis has greater genetic capacity to perform HMO metabolism reactions compared to other Bifidobacteria. Other Bifidos can metabolize HMOs but may do so less efficiently or require cooperation between different bacteria to perform different steps of the pathway.

The gene clusters that are more prevalent in infantis are also more prevalent in the TA samples (FIG. 24). They are clusters Hi (Blon_2331-2361), H2 (Blon_0243-Blon_0248), H3 (Blon_0247 Blon_0244-Blon_0248), H4 (Blon0625; Blon_0641-Blon_0651), and Urease (Blon_0104-Blon0115). (“Blon_----” are gene names for_B_ifidobacterium_lon_gum.)

TABLE 16
HMO Gene List. H5 genes (not specific to infantis) are italicized.

| Ensembl Bacteria Gene ID | Blon_list | Cluster |
|---|---|---|
| ACJ53389 | Blon_2331 | H1 |
| ACJ53390 | Blon_2332 | H1 |
| ACJ53392 | Blon_2334 | H1 |
| ACJ53394 | Blon_2336 | H1 |
| ACJ53400, ACJ53403 | Blon_2342 Blon_2345 | H1 |
| ACJ53401, ACJ53404 | Blon_2343 Blon_2346 | H1 |
| ACJ53402 | Blon_2344 | H1 |
| ACJ53405 | Blon_2347 | H1 |
| ACJ53406 | Blon_2348 | H1 |
| ACJ53408 | Blon_2350 | H1 |
| ACJ53409 | Blon_2351 | H1 |
| ACJ53410 | Blon_2352 | H1 |
| ACJ53412 | Blon_2354 | H1 |
| ACJ53413 | Blon_2355 | H1 |
| ACJ53415 | Blon_2357 | H1 |
| ACJ53417 | Blon_2359 | H1 |
| ACJ53418 | Blon_2360 | H1 |
| ACJ53419 | Blon_2361 | H1 |
| ACJ51372 | Blon_0243 | H2 |
| ACJ51373 | Blon_0244 | H2 |
| ACJ51374 | Blon_0245 | H2 |
| ACJ51376 | Blon_0248 | H2 |
| ACJ51375, ACJ51545 | Blon_0247 Blon_0425 | H3 |
| ACJ51544 | Blon_0423 | H3 |
| ACJ51546 | Blon_0426 | H3 |
| ACJ51732 | Blon_0625 | H4 |
| ACJ51748 | Blon_0641 | H4 |
| ACJ51749 | Blon_0642 | H4 |
| ACJ51750 | Blon_0643 | H4 |
| ACJ51751 | Blon_0644 | H4 |
| ACJ51752 | nanE Blon_0645 | H4 |
| ACJ51753 | Blon_0646 | H4 |
| ACJ51754 | Blon_0647 | H4 |
| ACJ51755 | Blon_0648 | H4 |
| ACJ51756 | Blon_0649 | H4 |
| ACJ51757 | Blon_0650 | H4 |
| ACJ51758 | Blon_0651 | H4 |
| ACJ53232 | Blon_2171 | H5 |
| ACJ53233 | galT Blon_2172 | H5 |
| ACJ53234 | Blon_2173 | H5 |
| ACJ53235 | Blon_2174 | H5 |
| ACJ53236 | Blon_2175 | H5 |
| ACJ53237 | Blon_2176 | H5 |
| ACJ53238 | Blon_2177 | H5 |
| ACJ51233 | Blon_0104 | Urease |
| ACJ51234 | Blon_0105 | Urease |
| ACJ51235 | Blon_0106 | Urease |
| ACJ51236 | Blon_0107 | Urease |
| ACJ51237 | Blon_0108 | Urease |
| ACJ51238 | Blon_0109 | Urease |
| ACJ51239 | Blon_0110 | Urease |
| ACJ51240 | ureC Blon_0111 | Urease |
| ACJ51241 | ureE Blon_0112 BLIJ_0113 | Urease |
| ACJ51242 | ureF Blon_0113 | Urease |
| ACJ51243 | ureG Blon_0114 | Urease |
| ACJ51244 | ureD Blon_0115 | Urease |

A subsequent cross-validated machine learning analysis was also used to identify metabolites and lipids associated with level of Bifidobacterium longum (rCLR transformed) or total Bifidobacterium genus (rCLR). “High” and “Low” were determined by dividing rCLR values into two quantiles around the median across all 116 profiled metagenomics samples. Only features with a confident identification were used for this analysis to improve interpretability of the results. The union of the top 25 metabolite features prioritized by elastic net and random forest for each of species-level B. longum or genus-level Bifidobacterium is given in the tables below. A blank cell for “Associated with . . . ” means the feature was not prioritized by the machine learning analysis for that outcome.

TABLE 17
Stool metabolites for distinguishing high from low B. longum or total Bifidobacterium

| | Associated | | | | | |
|---|---|---|---|---|---|---|
| | with B. | Associated | | | | |
| Unique | longum | with total genus | | | | |
| metabolite | level | Bifidobacterium | | | Retention | Molecular |
| ID | (rCLR) | (rCLR) | Identification | HMDB ID | Time | Weight |
| s2met_67 | High | High | 4-Hydroxyphenyllactic | HMDB0000755 | 10.097 | 182.0572 |
| | | | acid | | | |
| s2met_19 | High | High | 5′-S-Methyl-5′- | HMDB0001173 | 3.093 | 297.08963 |
| | | | thioadenosine | | | |
| s2met_34 | High | High | Adenine | HMDB0000034 | 6.364 | 135.05405 |
| s2met_73 | High | High | Alanylalanine | HMDB0028680 | 11.035 | 160.08452 |
| s2met_54 | High | High | Cytosine | HMDB0000630 | 8.497 | 111.04364 |
| s2met_15 | High | High | Dehydrocholic acid | HMDB0304121; | 2.549 | 402.24026 |
| | | | | HMDB0000502 | | |
| s2met_82 | High | High | gamma- | HMDB0034367 | 12.229 | 278.09357 |
| | | | Glutamylmethionine | | | |
| s2met_59 | High | High | Indole-3-lactic acid | HMDB0000671 | 9.004 | 205.0733 |
| s2met_91 | High | High | L-Citrulline | HMDB0000904 | 13.141 | 175.09567 |
| s2met_95 | High | High | N-Acetyl-DL-glutamic | HMDB0001138 | 14.201 | 189.0631 |
| | | | acid | | | |
| s2met_81 | High | High | N-Alpha-acetyllysine | HMDB0000446 | 12.161 | 188.11613 |
| s2met_92 | High | High | S-Adenosylmethionine | HMDB0001185 | 13.605 | 398.13746 |
| s2met_112 | High | High | Sugar acid | | 14.576 | 194.04202 |
| | | | 6C_RT14.576 | | | |
| s2met_16 | High | Low | Bilirubin | HMDB0000054; | 2.866 | 584.26343 |
| | | | | HMDB0240584; | | |
| | | | | HMDB0000488 | | |
| s2met_29 | High | | 2′-Deoxyadenosine | HMDB0000101 | 4.65 | 251.10196 |
| s2met_35 | High | | 3-Phenyllactic acid | HMDB0000748; | 6.435 | 166.06223 |
| | | | | HMDB0000779; | | |
| | | | | HMDB0000563 | | |
| s2met_23 | High | | 4-Pyridoxic acid | HMDB0000017 | 3.633 | 183.05256 |
| s2met_85 | High | | 5-Aminovaleric acid | HMDB0003355 | 12.526 | 117.07934 |
| s2met_33 | High | | Alpha-Ketovaline | HMDB0000019 | 6.036 | 116.04641 |
| s2met_84 | High | | gamma-Aminobutyric | HMDB0000112 | 12.42 | 103.06379 |
| | | | acid (GABA) | | | |
| s2met_106 | High | | Hexosamine | | 11.625 | 179.07951 |
| s2met_52 | High | | Kynurenic acid | HMDB0000715 | 8.371 | 189.04201 |
| s2met_51 | High | | Levulinic acid | HMDB0000720 | 8.269 | 116.04641 |
| s2met_111 | High | | N- | | 13.797 | 301.04642 |
| | | | Acetylhexosamine_ | | | |
| | | | RT13.797 | | | |
| s2met_38 | High | | N-Acetylisoleucine OR | HMDB0061684; | 6.945 | 173.10451 |
| | | | N-Acetylleucine | HMDB0011756 | | |
| s2met_83 | High | | N-Acetylornithine | HMDB0003357 | 12.375 | 174.10024 |
| s2met_62 | High | | N-Acetylputrescine | HMDB0002064 | 9.423 | 130.11083 |
| s2met_78 | High | | N-alpha-L-Acetyl- | HMDB0004620 | 11.992 | 216.12238 |
| | | | arginine | | | |
| s2met_72 | High | | Nicotinamide | HMDB0001406 | 10.972 | 122.0483 |
| s2met_70 | Low | High | Acetyl-beta- | HMDB0015654 | 10.54 | 159.12605 |
| | | | methylcholine | | | |
| s2met_74 | Low | High | L-Tyrosine | HMDB0000158 | 11.225 | 181.07387 |
| s2met_44 | Low | Low | 6-Methylquinoline | HMDB0033115 | 7.614 | 143.07361 |
| s2met_36 | Low | Low | Creatinine | HMDB0000562 | 6.663 | 113.05929 |
| s2met_8 | Low | Low | FA 12:0 | HMDB0000638 | 1.861 | 200.17708 |
| s2met_9 | Low | | 9-HpODE | HMDB0242602 | 1.92 | 312.22994 |
| s2met_50 | Low | | Acamprosate | HMDB0014797 | 8.228 | 181.0402 |
| s2met_100 | Low | | DL-Malic acid | HMDB0000156; | 15.601 | 134.02061 |
| | | | | HMDB0031518 | | |
| s2met_6 | Low | | FA 18:2 | HMDB0005048; | 1.612 | 280.2401 |
| | | | | HMDB0003797; | | |
| | | | | HMDB0000673; | | |
| | | | | HMDB0005047 | | |
| s2met_109 | Low | | Hexose | | 13.41 | 180.06272 |
| s2met_40 | Low | | Lenticin | HMDB0061115 | 7.032 | 246.13695 |
| s2met_63 | Low | | Xanthine | HMDB0000292 | 9.45 | 152.0326 |
| s2met_57 | | High | 3-Hydroxybutyric Acid | HMDB0000011; | 8.792 | 104.04638 |
| | | | | HMDB0000442 | | |
| s2met_55 | | High | Betaine | HMDB0000043 | 8.649 | 117.07933 |
| s2met_43 | | High | Choline | HMDB0000097 | 7.469 | 103.10018 |
| s2met_71 | | High | DL-Carnitine | HMDB0000062 | 10.615 | 161.10532 |
| s2met_102 | | High | L-Arginine | HMDB0000517 | 17.771 | 174.11175 |
| s2met_93 | | High | L-Glutamic acid | HMDB0000148 | 13.803 | 147.05264 |
| s2met_87 | | High | L-Glutamine | HMDB0000641 | 12.691 | 146.06905 |
| s2met_80 | | High | L-Threonine | HMDB0000167 | 12.125 | 119.05778 |
| s2met_69 | | High | L-Valine | HMDB0000883 | 10.258 | 117.07933 |
| s2met_12 | | High | N-(5- | | 2.156 | 186.13699 |
| | | | acetamidopentyl) | | | |
| | | | acetamide | | | |
| s2met_97 | | High | N-Acetylaspartic acid | HMDB0000812 | 14.431 | 175.04735 |
| s2met_103 | | High | N- | | 9.787 | 221.08947 |
| | | | Acetylhexosamine_ | | | |
| | | | RT9.787 | | | |
| s2met_14 | | High | Palmitoylcarnitine | HMDB0000222; | 2.396 | 399.33508 |
| | | | | HMDB0240774; | | |
| | | | | HMDB0240783 | | |
| s2met_110 | | High | Sugar acid | | 13.449 | 194.04204 |
| | | | 6C_RT13.449 | | | |
| s2met_7 | | High | Testosterone sulfate | HMDB0002833 | 1.835 | 368.16544 |
| s2met_25 | | Low | 3,4-Dimethylbenzoic | HMDB0002237 | 3.914 | 150.06727 |
| | | | acid | | | |
| s2met_2 | | Low | FA 20:2 | HMDB0061864; | 1.558 | 308.27134 |
| | | | | HMDB0005060 | | |
| s2met_79 | | Low | Glyceric acid | HMDB0000139; | 12.007 | 106.02564 |
| | | | | HMDB0006372 | | |
| s2met_60 | | Low | L-Phenylalanine | HMDB0000159 | 9.07 | 165.07874 |
| s2met_65 | | Low | L-Tryptophan | HMDB0000929 | 9.941 | 204.08986 |
| s2met_61 | | Low | Methylnicotinamide | HMDB0059711; | 9.392 | 136.06381 |
| | | | | HMDB0000699; | | |
| | | | | HMDB0003152; | | |
| | | | | HMDB0246826 | | |
| s2met_31 | | Low | Phenethylamine | HMDB0012275 | 5.742 | 121.08944 |
| s2met_28 | | Low | Serotonin | HMDB0000259 | 4.549 | 176.09507 |

TABLE 18
Stool lipids for distinguishing high from low B. longum or total Bifidobacterium

| | | Top | | | | | |
|---|---|---|---|---|---|---|---|
| | Top | feature for | | | | | |
| | feature for | total | | | | | |
| | B. longum | genus | | | | | |
| Unique | level | Bifidobacterium | | | Retention. | | |
| lipid ID | (rCLR) | (rCLR) | Identification | Lipid.Class | Time..min. | Quant.Ion | Polarity |
| s2lip_327 | High | High | Cer[AS] d18:2_24:0 | Cer[AS] | 12.776 | 662.61029 | − |
| s2lip_1405 | High | | (2E,4E,14E)-13-Hydroperoxy- | FAA | 4.962 | 394.33173 | + |
| | | | N-(2-methylpropyl)icosa- | | | | |
| | | | 2,4,14-trienamide | | | | |
| s2lip_42 | High | | AC 17:1 (s2lip_42) | AC | 1.814 | 412.34161 | + |
| s2lip_2935 | High | | Arachidonic acid | FA | 8.631 | 305.24744 | + |
| s2lip_413 | High | | CE 20:4 | CE | 16.65 | 690.61957 | + |
| s2lip_69 | High | | Cer[NS] d36:3 (s2lip_69) | Cer[NS] | 7.48 | 562.51965 | + |
| s2lip_2869 | High | | Docosahexaenoic acid (DHA) | FA | 8.546 | 329.24762 | + |
| s2lip_330 | High | | HexCer[NS] d18:1_24:0 | HexCer[NS] | 12.84 | 810.68359 | − |
| s2lip_138 | High | | HexCer[NS] d36:3 | HexCer[NS] | 8.85 | 724.57214 | + |
| s2lip_87 | High | | PC 32:2 | PC | 8.089 | 730.5387 | + |
| s2lip_81 | High | | PE 36:2 | PE | 7.977 | 742.53992 | − |
| s2lip_82 | High | | PG 18:0_22:6 | PG | 7.985 | 821.53497 | − |
| s2lip_63 | High | | SHexCer d34:2 | SHexCer | 7.033 | 776.49957 | − |
| s2lip_401 | High | | TG 20:3_22:4_22:4 | TG | 16.432 | 1026.85168 | + |
| s2lip_36 | Low | Low | LysoPE 16:0 | LysoPE | 1.668 | 452.27853 | − |
| s2lip_166 | Low | Low | PE 15:0_18:1 | PE | 9.198 | 702.50873 | − |
| s2lip_187 | Low | Low | PE 16:0_17:1 | PE | 9.51 | 702.50928 | − |
| s2lip_204 | Low | Low | PE 16:0_18:1 (s2lip_204) | PE | 9.794 | 718.53839 | + |
| s2lip_156 | Low | Low | PE 16:0_18:2 | PE | 9.089 | 714.50909 | − |
| s2lip_145 | Low | Low | PE 32:1 | PE | 8.917 | 688.49292 | − |
| s2lip_171 | Low | Low | PE 34:2 (s2lip_171) | PE | 9.256 | 716.52295 | + |
| s2lip_155 | Low | Low | PE 36:5 | PE | 9.068 | 738.50354 | + |
| s2lip_94 | Low | Low | PG 16:0_17:1 | PG | 8.251 | 733.50378 | − |
| s2lip_210 | Low | Low | Plasmenyl-PC P-16:0_16:0 | Plasmenyl- | 9.96 | 776.58173 | − |
| | | | | PC | | | |
| s2lip_153 | Low | | Alkanyl-DG O-18:2_18:2 | Alkanyl-DG | 9.049 | 603.5351 | + |
| | | | (s2lip_153) | | | | |
| s2lip_194 | Low | | Alkanyl-DG O-34:3 | Alkanyl-DG | 9.584 | 577.5188 | + |
| | | | (s2lip_194) | | | | |
| s2lip_270 | Low | | Alkanyl-DG O-34:3 | Alkanyl-DG | 11.264 | 577.51978 | + |
| | | | (s2lip_270) | | | | |
| s2lip_349 | Low | | Cer[NS] d18:1_24:0 | Cer[NS] | 14.171 | 708.65216 | − |
| s2lip_316 | Low | | Cer[NS] d40:2 (s2lip_316) | Cer[NS] | 12.32 | 620.59821 | + |
| s2lip_318 | Low | | Cer[NS] d41:2 (s2lip_318) | Cer[NS] | 12.362 | 692.6214 | − |
| s2lip_5944 | Low | | cis-12-Octadecenoic acid | FA | 12.134 | 297.27847 | + |
| | | | methyl ester | | | | |
| s2lip_1103 | Low | | Linoleoyl ethanolamide | FAA | 2.601 | 324.2897 | + |
| s2lip_8 | Low | | LysoPG 18:2 | LysoPG | 1.106 | 507.27301 | − |
| s2lip_35 | Low | | LysoPI 18:0 | LysoPI | 1.62 | 599.32062 | − |
| s2lip_14 | Low | | LysoPI 18:1 | LysoPI | 1.197 | 597.30585 | − |
| s2lip_85 | Low | | PE 28:0 | PE | 8.005 | 636.45966 | + |
| s2lip_90 | Low | | PE 30:1 | PE | 8.116 | 660.46185 | − |
| s2lip_71 | Low | | PG 16:1_16:0 | PG | 7.539 | 719.48798 | − |
| s2lip_61 | Low | | SP d18:1 (s2lip_61) | SP | 3.545 | 300.28958 | + |
| s2lip_27 | | High | AC 20:3 | AC | 1.479 | 450.3577 | + |
| s2lip_23 | | High | AC 22:6 (s2lip_23) | AC | 1.36 | 472.34222 | + |
| s2lip_41 | | High | AC 22:6 (s2lip_41) | AC | 1.787 | 472.34149 | + |
| s2lip_105 | | High | Cer[NS] d34:2 (s2lip_105) | Cer[NS] | 8.354 | 536.50433 | + |
| s2lip_251 | | High | Cer[NS] d36:0 (s2lip_251) | Cer[NS] | 10.655 | 550.55536 | + |
| s2lip_389 | | High | Cer[NS] d36:1 (s2lip_389) | Cer[NS] | 16.035 | 548.54065 | + |
| s2lip_152 | | High | HexCer[NS] d18:2_18:0 | HexCer[NS] | 8.987 | 724.57458 | − |
| s2lip_207 | | High | HexCer[NS] d36:1 | HexCer[NS] | 9.821 | 728.60339 | + |
| s2lip_245 | | High | HexCer[NS] d40:3 (s2lip_245) | HexCer[NS] | 10.516 | 780.63568 | + |
| s2lip_12 | | High | LysoPG 16:0 (s2lip_12) | LysoPG | 1.186 | 483.27313 | − |
| s2lip_25 | | High | LysoPG 18:1 | LysoPG | 1.405 | 509.2887 | − |
| s2lip_231 | | High | PC 33:0 | PC | 10.242 | 748.59351 | + |
| s2lip_263 | | High | PC 35:1 (s2lip_263) | PC | 10.961 | 774.60895 | + |
| s2lip_79 | | High | PC 42:10 | PC | 7.863 | 854.5827 | + |
| s2lip_62 | | High | PG 22:6_22:6 | PG | 6.9 | 865.50311 | − |
| s2lip_89 | | High | PG 36:2 (s2lip_89) | PG | 8.112 | 773.53448 | − |
| s2lip_266 | | High | Plasmanyl-PC O-38:2 | Plasmanyl- | 11.152 | 800.65381 | + |
| | | | | PC | | | |
| s2lip_375 | | High | TG 54:6 (s2lip_375) | TG | 15.718 | 896.7702 | + |
| s2lip_205 | | Low | Cer[NS] d36:2 (s2lip_205) | Cer[NS] | 9.812 | 546.52563 | + |
| s2lip_80 | | Low | Cer[NS] d36:2 (s2lip_80) | Cer[NS] | 7.87 | 564.53558 | + |
| s2lip_34 | | Low | LysoPC 20:3 | LysoPC | 1.608 | 546.3551 | + |
| s2lip_9 | | Low | LysoPE 14:0 | LysoPE | 1.117 | 424.24725 | − |
| s2lip_75 | | Low | PE 16:0_16:0 | PE | 7.793 | 690.5036 | − |
| s2lip_98 | | Low | PE 29:0 | PE | 8.277 | 648.46161 | − |
| s2lip_161 | | Low | PE 31:0 | PE | 9.119 | 678.50629 | + |
| s2lip_160 | | Low | PE-NMe 30:0 | PE-NMe | 9.117 | 676.49347 | − |
| s2lip_225 | | Low | PE-NMe2 17:1_17:1 | PE-NMe2 | 10.18 | 742.54071 | − |
| s2lip_114 | | Low | PG 18:1_18:1 | PG | 8.481 | 773.5354 | − |
| s2lip_233 | | Low | Plasmenyl-PE P-34:1 | Plasmenyl- | 10.274 | 700.52997 | − |
| | | | | PE | | | |
| s2lip_243 | | Low | SM d38:1 | SM | 10.501 | 759.63776 | + |

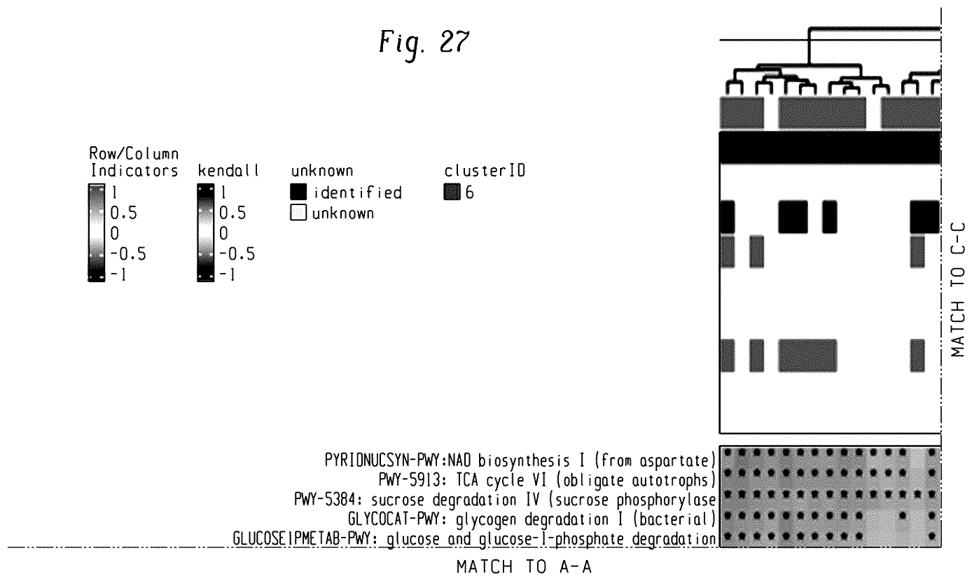

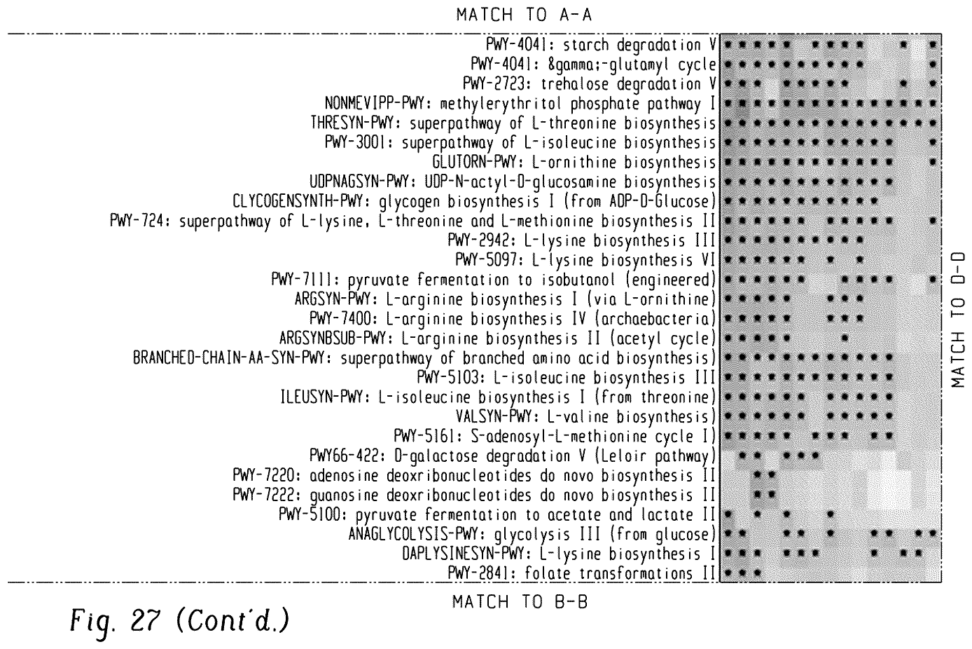

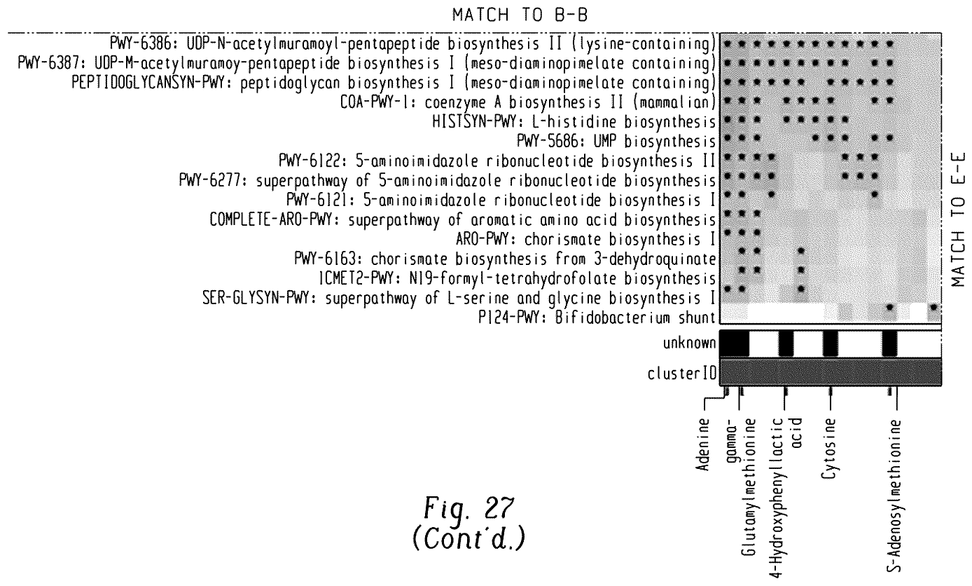

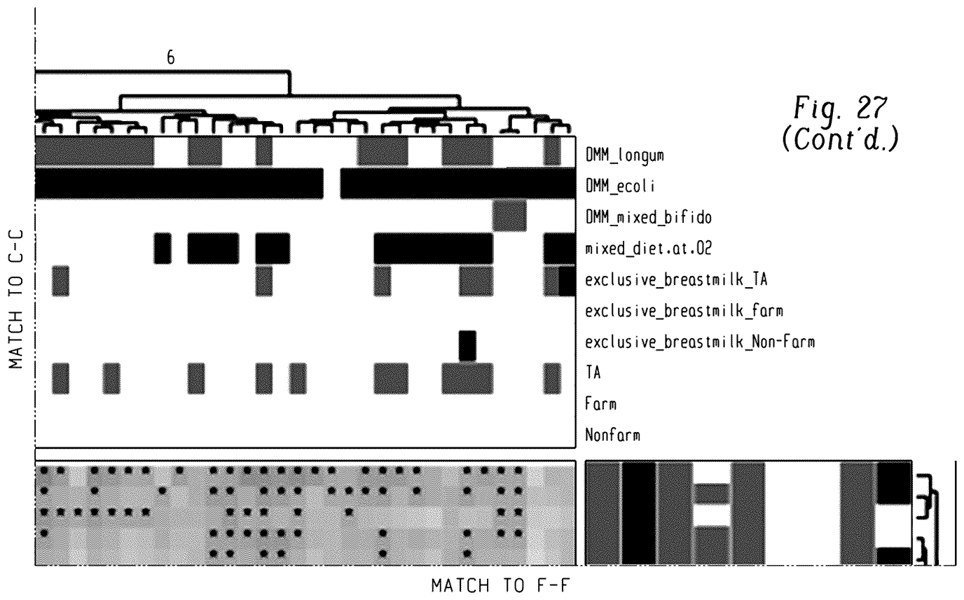

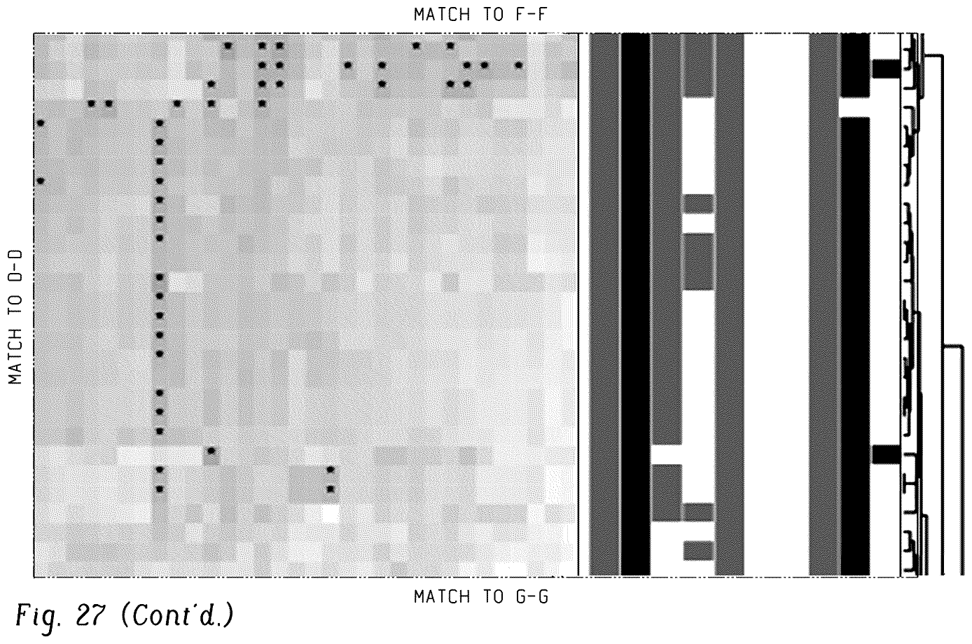

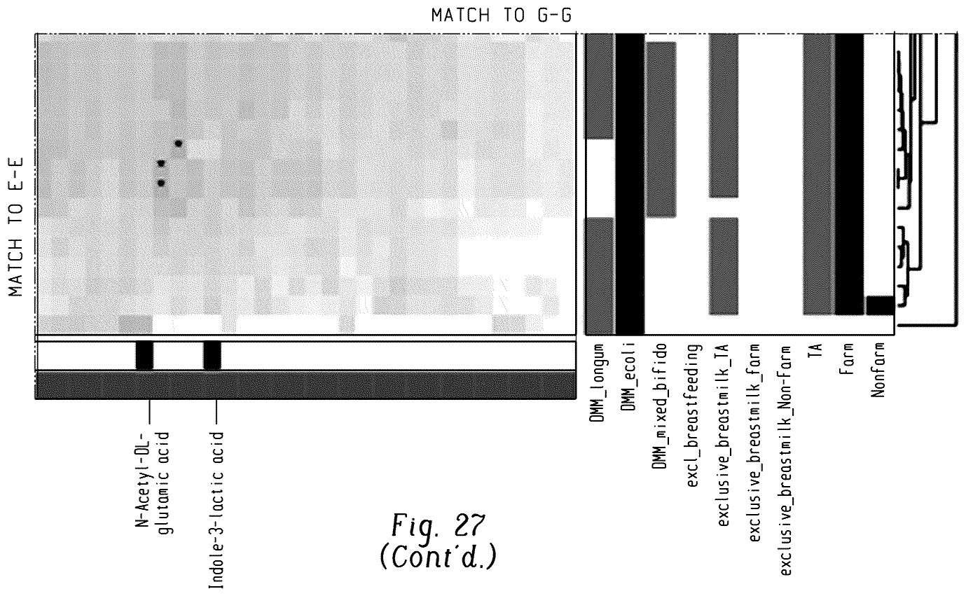

FIG. 27 illustrates correlated module of 2mo stool microbial pathway capacity and measured metabolites that are associated with Bifidobacterium longum-dominated microbiome and TA status. Partial correlations between microbial pathways and stool metabolites. * adjusted p<0.05. Partial Kendall tau correlations were computed between two month stool microbial metabolic pathway capacity (rows) and metabolites measured with respect to farm status (aiming to mitigate spurious correlations due to a third variable, farm status). The resulting correlation matrix was filtered to rows and columns with at least three significant correlations. Then, modules were identified by hierarchical clustering on the metabolites and manually identifying modules enriched for farm differences. This module is enriched for microbial pathways that are elevated in TA infants and metabolites that are associated with Bifidobacterium longum dominated microbiomes (DMM_longum), TA status, or exclusive breastfeeding. Identified metabolites are indicated in purple and given names on the bottom.

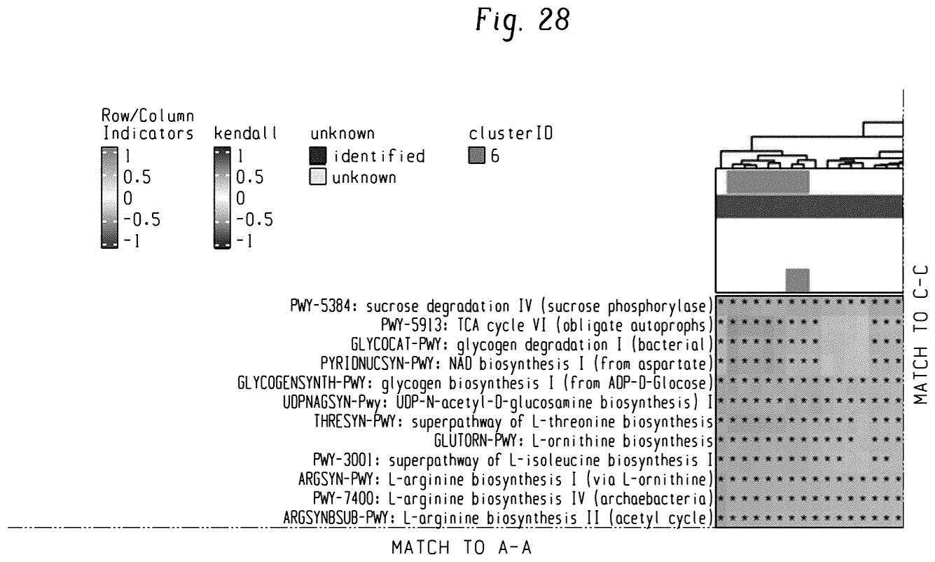

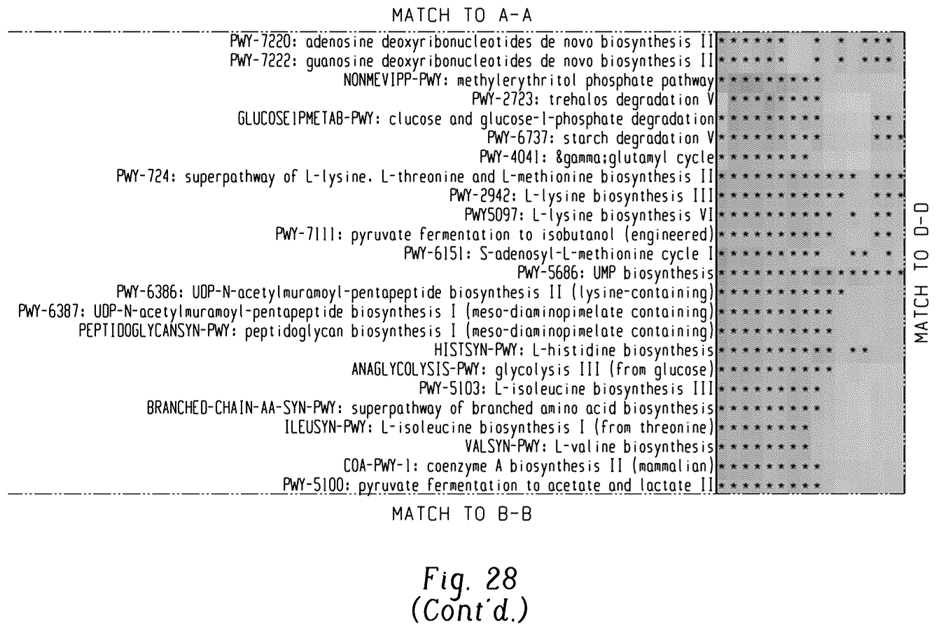

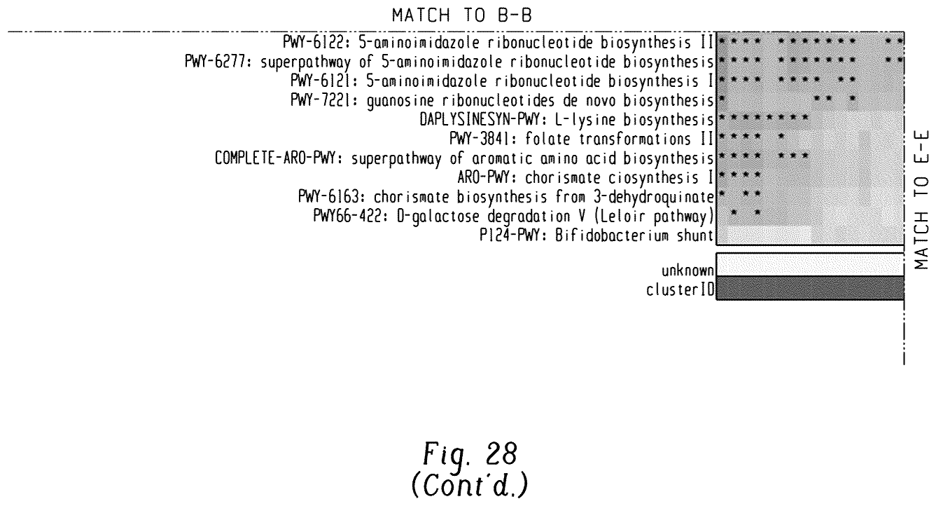

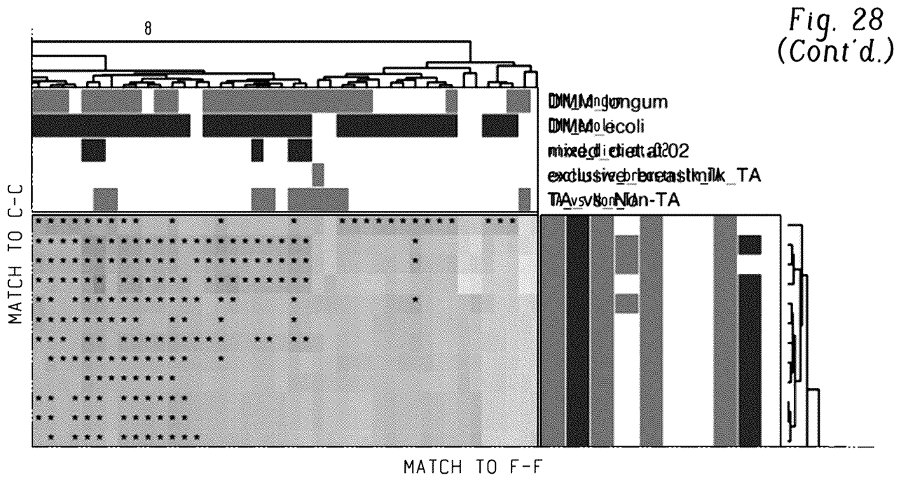

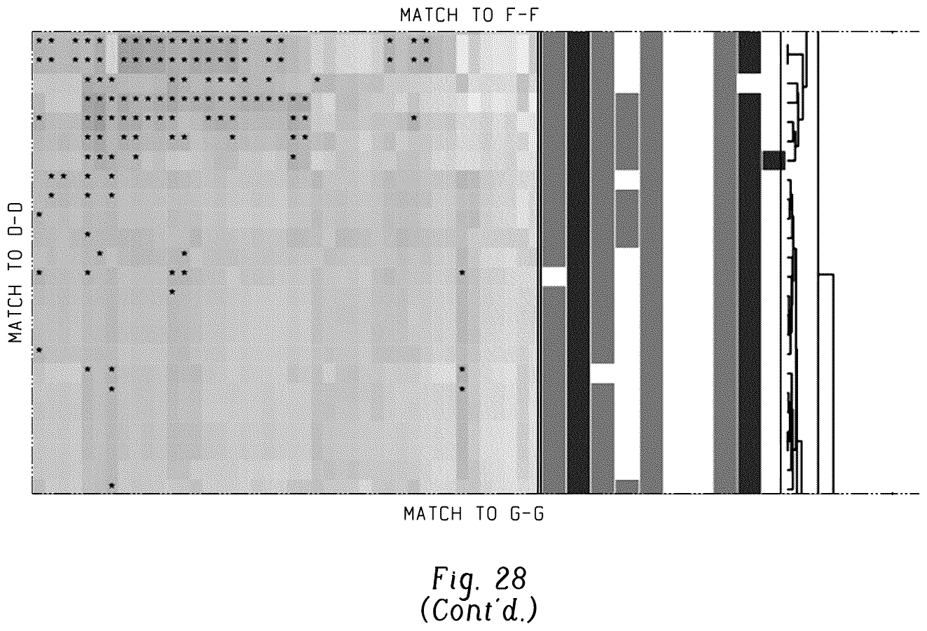

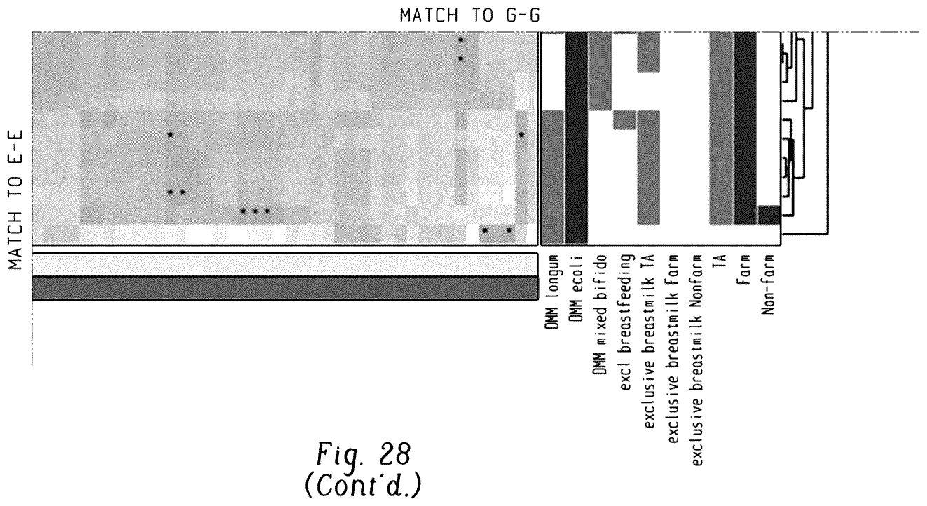

FIG. 28 illustrates correlated module of 2mo stool microbial pathway capacity and measured lipids that are associated with Bifidobacterium longum-dominated microbiome and TA status. Partial correlations between microbial pathways and stool lipids. * adjusted p<0.05. Partial Kendall tau correlations were computed between two month stool microbial metabolic pathway capacity (rows) and measured lipids with respect to farm status (aiming to mitigate spurious correlations due to a third variable, farm status). The resulting correlation matrix were filtered to rows and columns with at least three significant correlations. Modules were identified by hierarchical clustering on the lipids and manual selection for modules enriched for farm-related differences. This module is enriched for microbial pathways that are elevated in TA infants and lipids that are associated with Bifidobacterium longum dominated microbiomes (DMM_longum) or TA status.

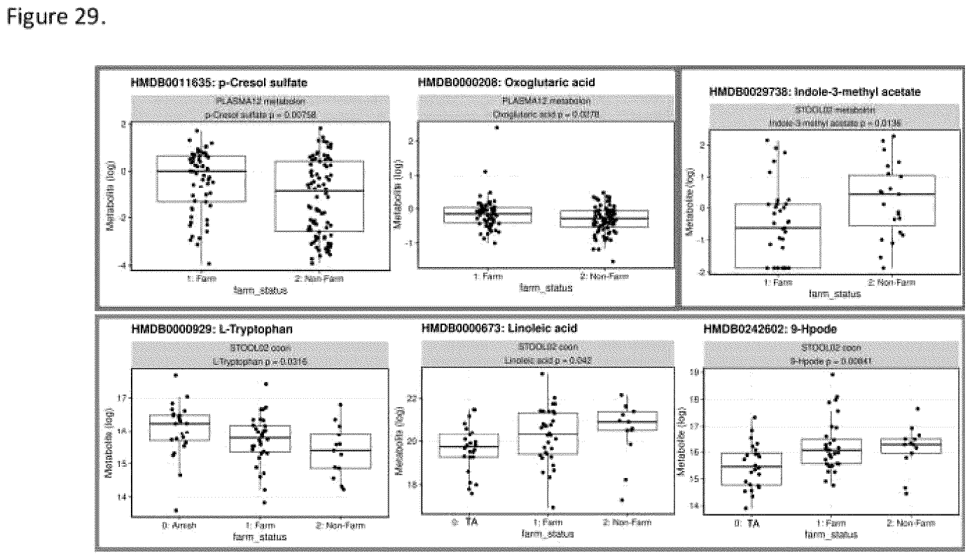

FIG. 29 illustrates farm status vs. selected tryptophan pathway metabolites (MW or KW test). Top row is Metabolon data from PLASMA12 (blue) and STOOL02 (orange). Bottom Row is STOOL02. These metabolites can be used alone or in combination with the metagenomics data to predict future respiratory disease. Datasets PLASMA00: includes WFS and WISC; PLASMA12: WFS and WISC or WISC only. STOOL02: either WFS and WISC or WISC only.

Metabolites in the tryptophan pathway, starting with L-tryptophan are higher in TA (differences in kynurenine were identified in TA plasma at birth): Tryptophan (Trp) is an essential amino acid and is also the obligatory substrate for the production of several important bioactive substances. For example, tryptophan is a substrate for the synthesis of serotonin (5-hydroxytryptpamine, 5-HT) in the brain and gut, and melatonin in the pineal gland. In vertebrates, central 5-HT plays an integrative role in the behavioral and neuroendocrine stress response. Accordingly, effects of dietary Trp on the neuroendocrine stress response have been reported in a variety of species, spanning from teleosts to humans.

Linoleic acid (LA) is a polyunsaturated fatty acid (PUFA) precursor to the longer n-6 fatty acids commonly known as omega-6 fatty acids. An essential fatty acid, is metabolized to gamma linolenic acid (GLA), which serves as an important constituent of neuronal membrane phospholipids and also as a substrate for prostaglandin formation, seemingly important for preservation of nerve blood flow. This pathway leads to the production of 9-Hpode.

9-Hpode Hydroxyoctadecadienoic acids (HODEs) are stable oxidation products of linoleic acid, the generation of which is increased where oxidative stress is increased, such as in diabetes. In early atherosclerosis, 13-HODE is generated in macrophages by 15-lipoxygenase-1. This enhances protective mechanisms through peroxisome proliferator-activated receptor (PPAR)-g activation leading to increased clearance of lipid and lipid-laden cells from the arterial wall. In later atherosclerosis, both 9-HODE and 13-HODE are generated nonenzymatically. At this stage, early protective mechanisms are overwhelmed and pro-inflammatory effects of 9-HODE, acting through the receptor GPR132, and increased apoptosis predominate leading to a fragile, acellular plaque. Increased HODE levels thus contribute to atherosclerosis progression and the risk of clinical events such as myocardial infarction or stroke. Better understanding of the role of HODEs may lead to new pharmacologic approaches to modulate their production or action, and therefore lessen the burden of atherosclerotic disease in high-risk patients.

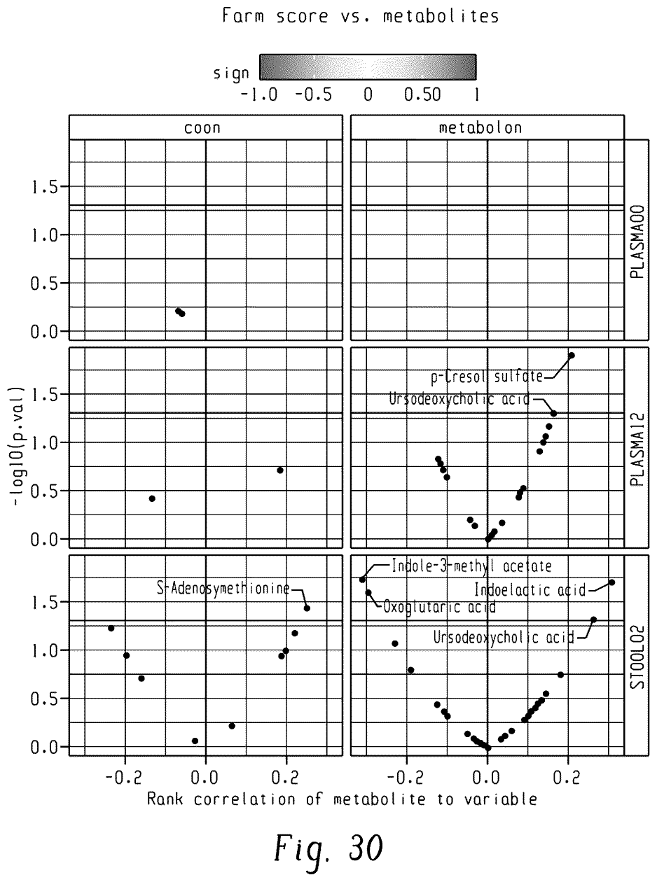

FIG. 30 shows farm score vs tryptophan metabolites. Farm score is a function of number and frequency of farm animal exposures. These metabolites can be used alone or in combination with the metagenomics data to predict future respiratory disease.

The metabolites identified herein can be used alone or in combination with the metagenomics data to predict future respiratory disease, or my be employed in a prebiotic or probiotic supplement to pregnant females, infants, toddlers or children under the age of 5 years old.

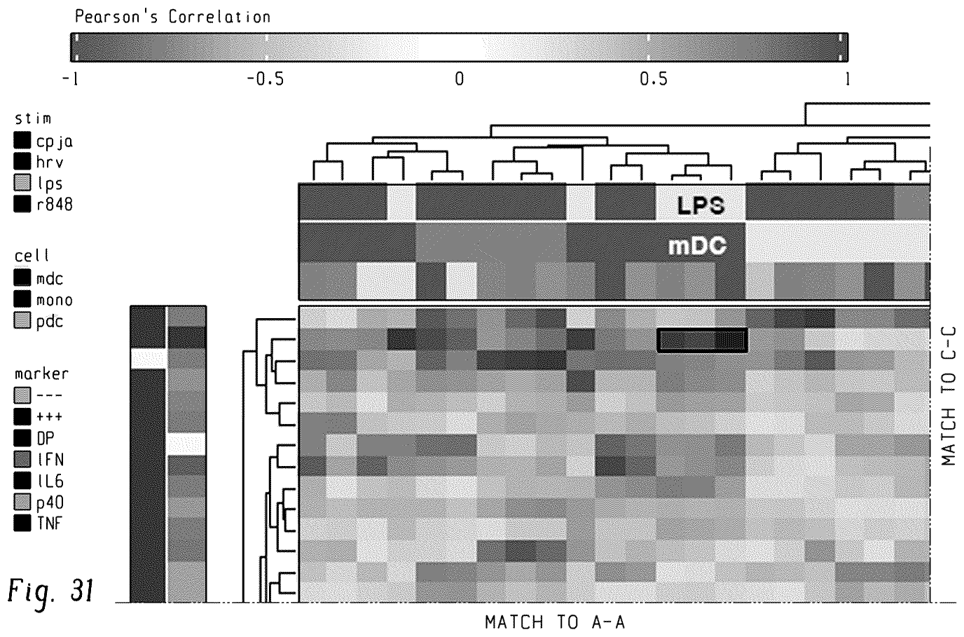

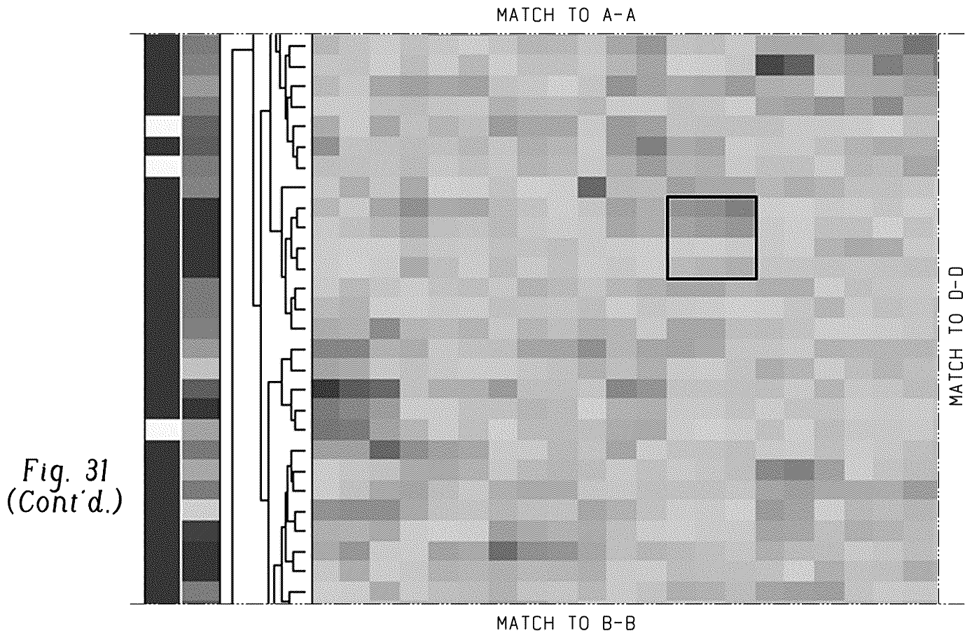

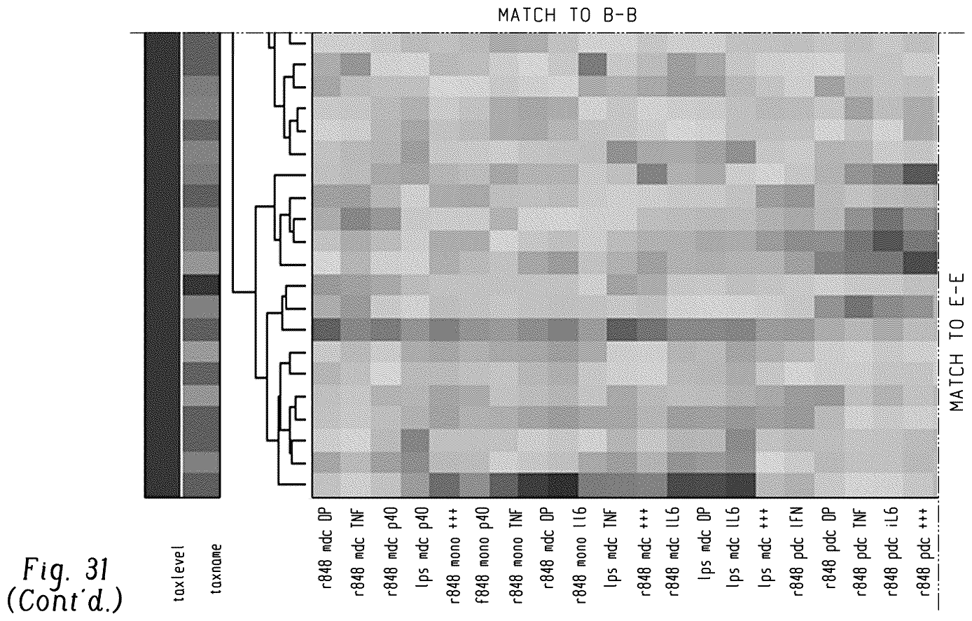

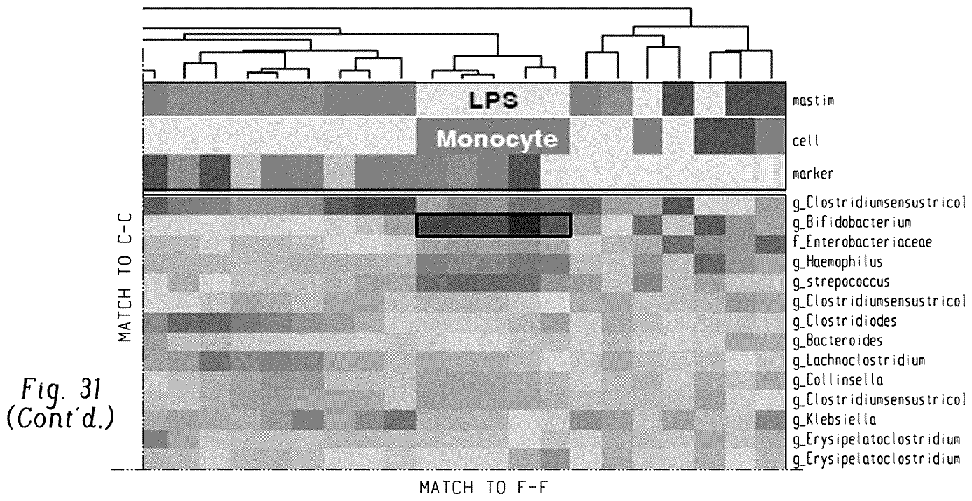

FIG. 31 shows microbiome-immune PLS regression. There is a stronger correlation between these Bifidobacterium amplicon sequence variants (ASVs) and lipopolysaccharide (LPS) monocyte responses, but not R848 monocyte responses.

FIG. 32 shows mixed effects model. Classified WISC infants as “high”, “medium”, or “low” Bifidobacterium based on 16S. Linear mixed effects model was used to make comparisons between groups at 1 yr and 2 yr. mDC response to LPS at 2 years of age was positively associated to Bifido abundance in early life

FIG. 33. PCA on STOOL02 metabolomics, lipidomics. Control samples in gray.

FIG. 34 illustrates microbe-metabolomics module in network form (edges for significant partial Kendall correlations). The map shows connections between pathway (squares) and metabolites (circles). The ones with a wider outline around the circles and squares indicate they are higher in TA.

In the comparative genomics analysis by LoCascio et al, specific gene clusters for HMO metabolism were found in infantis but not in longum subspecies. Infantis has greater genetic capacity to perform HMO metabolism reactions compared to other Bifidobacteria. Other Bifidos can metabolize HMOs but may do so less efficiently or require cooperation between different bacteria to perform different steps of the pathway.

The gene clusters that are more prevalent in infantis are also more prevalent in the TA samples (FIG. 24). They are clusters H1 (Blon_2331-2361), H2 (Blon_0243-Blon_0248), H3 (Blon_0247, Blon_0244-Blon_0248), H4 (Blon_0625; Blon_0641-Blon_0651), and Urease (Blon_0104-Blon_0115).

Infantis-specific human milk oligosaccharide (HMO) genes include but are not limited to HMO clusters H1 (Blon_2331-2361), H2 (Blon_0243-Blon_0248), H3 (Blon_0247, Blon_0244-Blon_0248), H4 (Blon_0625; Blon_0641-Blon_0651), and Urease (Blon_0104-Blon_0115). That is, the gene clusters that are more prevalent in infantis are also more prevalent in the TA samples (as shown in FIG. 24). They are clusters H1 (Blon_2331-2361), H2 (Blon_0243-Blon_0248), H3 (Blon_0247, Blon_0244-Blon_0248), H4 (Blon_0625; Blon_0641-Blon_0651), and Urease(Blon_0104-Blon_0115).

### REFERENCES

Alfvén, T., C. Braun-Fahrlander, B. Brunekreef, E. von Mutius, J. Riedler, A. Scheynius, M. van Hage, et al. 2006. “Allergic Diseases and Atopic Sensitization in Children Related to Farming and Anthroposophic Lifestyle—the PARSIFAL Study.” Allergy6l (4): 414-21.
Arrieta, Marie-Claire, Leah T. Stiemsma, Pedro A. Dimitriu, Lisa Thorson, Shannon Russell, Sophie Yurist-Doutsch, Boris Kuzeljevic, et al. 2015. “Early Infancy Microbial and Metabolic Alterations Affect Risk of Childhood Asthma.” Science Translational Medicine7 (307): 307ra152.
Beghini, Francesco, Lauren J. McIver, Aitor Blanco-Miguez, Leonard Dubois, Francesco Asnicar, Sagun Maharjan, Ana Mailyan, et al. 2021. “Integrating Taxonomic, Functional, and Strain-Level Profiling of Diverse Microbial Communities with bioBakery 3.” eLifel0 (May). https://doi.org/10.7554/eLife.65088.
Davis, Jasmine C. C., Zachery T. Lewis, Sridevi Krishnan, Robin M. Bernstein, Sophie E. Moore, Andrew M. Prentice, David A. Mills, Carlito B. Lebrilla, and Angela M. Zivkovic. 2017. “Growth and Morbidity of Gambian Infants Are Influenced by Maternal Milk Oligosaccharides and Infant Gut Microbiota.” Scientific Reports7 (January): 40466.
DeAngelis, Kristen M., Eoin L. Brodie, Todd Z. DeSantis, Gary L. Andersen, Steven E. Lindow, and Mary K. Firestone. 2009. “Selective Progressive Response of Soil Microbial Community to Wild Oat Roots.” The ISME Journal3 (2): 168-78.
Ege, Markus J., Melanie Mayer, Anne-Cecile Normand, Jon Genuneit, William O. C. M. Cookson, Charlotte Braun-Fahrlander, Dick Heederik, Renaud Piarroux, Erika von Mutius, and GABRIELATransregio 22 Study Group. 2011. “Exposure to Environmental Microorganisms and Childhood Asthma.” The New England Journal of Medicine364 (8): 701-9.
Franzosa, Eric A., Lauren J. McIver, Gholamali Rahnavard, Luke R. Thompson, Melanie Schirmer, George Weingart, Karen Schwarzberg Lipson, et al. 2018. “Species-Level Functional Profiling of Metagenomes and Metatranscriptomes.” Nature Methods15 (11): 962-68.
Fujimura, Kei E., Alexandra R. Sitarik, Suzanne Havstad, Din L. Lin, Sophia Levan, Douglas Fadrosh, Ariane R. Panzer, et al. 2016. “Neonatal Gut Microbiota Associates with Childhood Multisensitized Atopy and T Cell Differentiation.” Nature Medicine22 (10): 1187-91.
Haahtela, T., T. Laatikainen, H. Alenius, P. Auvinen, N. Fyhrquist, I. Hanski, L. von Hertzen, et al. 2015. “Hunt for the Origin of Allergy—Comparing the Finnish and Russian Karelia.” Clinical and Experimental Allergy: Journal of the British Society for Allergy and Clinical Immunology45 (5): 891-901.
Hiippala, Kaisa, Veera Kainulainen, Maiju Suutarinen, Tuomas Heini, Jolene R. Bowers, Daniel Jasso-Selles, Darrin Lemmer, et al. 2020. “Isolation of Anti-Inflammatory and Epithelium Reinforcing Bacteroides and Parabacteroides Spp. from A Healthy Fecal Donor.” Nutrients12 (4). doi.org/10.3390/nu12040935.
Jatzlauk, G., S. Bartel, H. Heine, M. Schloter, and S. Krauss-Etschmann. 2017. “Influences of Environmental Bacteria and Their Metabolites on Allergies, Asthma, and Host Microbiota.” Allergy72 (12): 1859-67.
Kuhn, Max, and Hadley Wickham. 2020. “Tidymodels: A Collection of Packages for Modeling and Machine Learning Using Tidyverse Principles.”
LoCascio, Riccardo G., Prerak Desai, David A. Sela, Bart Weimer, and David A. Mills. 2010. “Broad Conservation of Milk Utilization Genes in Bifidobacterium Longum Subsp. Infantis as Revealed by Comparative Genomic Hybridization.” Applied and Environmental Microbiology76 (22): 7373-81.
Mallick, Himel, Ali Rahnavard, Lauren J. McIver, Siyuan Ma, Yancong Zhang, Long H. Nguyen, Timothy L. Tickle, et al. 2021. “Multivariable Association Discovery in Population-Scale Meta-Omics Studies.” bioRxiv. doi.org/10.1101/2021.01.20.427420.
McMurdie, Paul J., and Susan Holmes. 2013. “Phyloseq: An R Package for Reproducible Interactive Analysis and Graphics of Microbiome Census Data.” PloS One8 (4): e61217.
Milani, Christian, Sabrina Duranti, Francesca Bottacini, Eoghan Casey, Francesca Turroni, Jennifer Mahony, Clara Belzer, et al. 2017. “The First Microbial Colonizers of the Human Gut: Composition, Activities, and Health Implications of the Infant Gut Microbiota.” Microbiology and Molecular Biology Reviews: MMBR 81 (4).
Mutius, Erika von, and Donata Vercelli. 2010. “Farm Living: Effects on Childhood Asthma and Allergy.” Nature Reviews. Immunology10 (12): 861-68.
Nishiyama et al., Environ. Appl. Microbiol., 86:e01464-020 (2020).
Riedler, J., C. Braun-Fahrlander, W. Eder, M. Schreuer, M. Waser, S. Maisch, D. Carr, et al. 2001. “Exposure to Farming in Early Life and Development of Asthma and Allergy: A Cross-Sectional Survey.” The Lancet358 (9288): 1129-33.
Rocha Martin, Vanesa Natalin, Clarissa Schwab, Lukasz Krych, Evelyn Voney, Annelies Geirnaert, Christian Braegger, and Christophe Lacroix. 2019. “Colonization of Cutibacterium Avidum during Infant Gut Microbiota Establishment.” FEMS Microbiology Ecology95 (1). doi.org/10.1093/femsec/fiy215.
Scholz, Matthias, Doyle V. Ward, Edoardo Pasolli, Thomas Tolio, Moreno Zolfo, Francesco Asnicar, Duy Tin Truong, Adrian Tett, Ardythe L. Morrow, and Nicola Segata. 2016. “Strain-Level Microbial Epidemiology and Population Genomics from Shotgun Metagenomics.” Nature Methods13 (5): 435-38.
Segata, Nicola, Jacques Izard, Levi Waldron, Dirk Gevers, Larisa Miropolsky, Wendy S. Garrett, and Curtis Huttenhower. 2011. “Metagenomic Biomarker Discovery and Explanation.” Genome Biology12 (6): R60.
Segata, Nicola, Levi Waldron, Annalisa Ballarini, Vagheesh Narasimhan, Olivier Jousson, and Curtis Huttenhower. 2012. “Metagenomic Microbial Community Profiling Using Unique Clade-Specific Marker Genes.” Nature Methods9 (8): 811-14.
Seppo, Antti E., Kevin Bu, Madina Jumabaeva, Juilee Thakar, Rakin A. Choudhury, Chloe Yonemitsu, Lars Bode, et al. 2021. “Infant Gut Microbiome Is Enriched with Bifidobacterium Longum Ssp. Infantis in Old Order Mennonites with Traditional Farming Lifestyle.” Allergy, April. doi.org/10.1111/all.14877.
Seroogy, Christine M., Jeffrey J. VanWormer, Brent F. Olson, Michael D. Evans, Tara Johnson, Deanna Cole, Kathrine L. Barnes, et al. 2019. “Respiratory Health, Allergies, and the Farm Environment: Design, Methods and Enrollment in the Observational Wisconsin Infant Study Cohort (WISC): A Research Proposal.” BMC Research Notes12 (1): 423.
Stein, Michelle M., Cara L. Hrusch, Justyna Gozdz, Catherine Igartua, Vadim Pivniouk, Sean E. Murray, Julie G. Ledford, et al. 2016. “Innate Immunity and Asthma Risk in TA and Hutterite Farm Children.” The New England Journal of Medicine375 (5): 411-21.
Stokholm, Jakob, Martin J. Blaser, Jonathan Thorsen, Morten A. Rasmussen, Johannes Waage, Rebecca K. Vinding, Ann-Marie M. Schoos, et al. 2018. “Maturation of the Gut Microbiome and Risk of Asthma in Childhood.” Nature Communications9 (1): 141.
Tantoco, Jamee C., Jordan Elliott Bontrager, Qianqian Zhao, James DeLine, and Christine M. Seroogy. 2018. “The TA Have Decreased Asthma and Allergic Diseases Compared with Old Order Mennonites.” Annals of Allergy, Asthma & Immunology: Official Publication of the American College of Allergy, Asthma, & Immunology121 (2): 252-53.e1.
Von Ehrenstein, O. S., E. Von Mutius, S. Illi, L. Baumann, O. Bohm, and R. von Kries. 2000. “Reduced Risk of Hay Fever and Asthma among Children of Farmers.” Clinical and Experimental Allergy: Journal of the British Society for Allergy and Clinical Immunology30 (2): 187-93.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method to detect immune health status in a human infant or child, comprising:
providing a stool sample from a human infant or child; and
determining in the sample i) the relative abundance of bacteria including two or more of Bacteroides, Bifidobacterium, or Blautia, ii) the relative abundance of bacteria including two or more of Bifidobacterium bifidum, Bifidobacterium breve, Bifidobacterium longum, or Bifidobacterium pseudocatenulatum, or iii) the relative abundance or expression of two or more of Blon_0915, Blon_2177, Blon_0625, Blon_0244, Blon_0248; Blon_0426, ureF, Blon_0113, ureC Blon_0111, ureE Blon_0112 BLIJ_0113, Blon_0642, Blon_2336, Blon_2344, or Blon_0650.

2. The method of claim 1, wherein a relative abundance of Bacteroides of >10%, of Bifidobacterium of <60% or of Blautia of >10% is indicative of an infant or child at increased risk of allergies or a relative abundance of Bacteroides of >8%, of Bifidobacterium of <65% or of Blautia of >2% is indicative of an infant or child at increased risk of allergies.

3. The method of claim 1, wherein a relative abundance of Bacteroides of <10%, of Bifidobacterium of >60% or of Blautia of <10% is indicative of an infant or child at decreased risk of allergies or Bacteroides of <10%, of Bifidobacterium of >65% or of Blautia of <2% is indicative of an infant or child at decreased risk of allergies.

4. The method of claim 1, wherein a relative abundance of Bifidobacterium bifidum of 10% or less, Bifidobacterium breve of 25% or less, Bifidobacterium longum of 25% or greater, or of Bifidobacterium pseudocatenulatum of less than 2% is indicative of immune health in the infant or child or of Bifidobacterium breve of 15% or less, Bifidobacterium longum of 65% or greater, or of Bifidobacterium pseudocatenulatum of less than 3% is indicative of immune health in the infant or child.

5. The method of claim 1, wherein a relative abundance of Bifidobacterium bifidum of less than 5%, Bifidobacterium breve of greater than 20%, Bifidobacterium longum of less than 50%, or of Bifidobacterium pseudocatenulatum of greater than 2% is indicative of impaired immune health in the infant or child or of Bifidobacterium breve of greater than 15%, Bifidobacterium longum of less than 30%, or of Bifidobacterium pseudocatenulatum of greater than 3% is indicative of impaired immune health in the infant or child.

6. The method of claim 1, wherein an increase in the relative abundance of expression of two or more of Blon_0915, Blon_2171, Blon_2173, Blon_2334, galT Blon_2172, Blon_0244, Blon_0248; Blon_0426, ureF Blon_0113, ureC Blon_0111, ureE Blon_0112 BLIJ_0113, Blon_0642, Blon_2336, Blon_2344, or Blon_0650 is indicative of immune health in the infant or child.

7. The method of claim 1, wherein the sample is from a newborn to a 3 month old infant.

8. The method of claim 1, wherein the sample is from a 3 month old to a 9 month old infant.

9. The method of claim 1, wherein the sample is from an infant or child treated with a drug.

10. The method of claim 1, wherein the infant or child has necrotizing enterocolitis.

11. The method of claim 1, further comprising administering to the mother of the infant or child, or a pregnant mother a prebiotic or a probiotic.

12. The method of claim 11, wherein the prebiotic or probiotic comprises one or more bacteria, one or more antibodies, or one or more molecules that enhance the relative abundance of Bifidobacterium longum.

13. The method of claim 1, wherein the sample is analyzed using a nucleic acid amplification reaction.

14. The method of claim 1, wherein the sample is analyzed using genome sequencing.

15. A method to identify a human infant or child at higher risk of developing allergies as an adolescent or adult, comprising:
providing a stool sample from a human infant or child; and
determining in the sample i) the relative abundance of bacteria including two or more of Bacteroides, Bifidobacterium, or Blautia, ii) the relative abundance of bacteria including two or more of Bifidobacterium bifidum, Bifidobacterium breve, Bifidobacterium longum, or Bifidobacterium pseudocatenulatum, or iii) the relative abundance or expression of two or more of Blon_0915, Blon_2177, Blon_0625, Blon_0244, Blon_0248; Blon_0426, ureF Blon_0113, ureC Blon_0111, ureE Blon_0112 BLIJ_0113, Blon_0642, Blon_2336, Blon_2344, or Blon_0650.

16. The method of claim 15, further comprising administering to the infant or child at higher risk of developing allergies a composition comprising one or more prebiotics or one or more probiotics comprising Bifidobacterium infantis, Bifidobacterium longum, Bifidobacterium breve, and/or Bifidobacterium bifidum, or combinations thereof.

17. A method to enhance immune health comprising administering to a pregnant female, infant or child having or at risk of compromised immune health, an effective amount of a composition comprising i) a plurality of: one or more B vitamins, one or more short chain fatty acids, linoleic caid, linolenic acid, tryptophan, one or more tryptophan metabolites, indole-3-methylacetate, or one or more hydroxyoctadecadienoic acids, or combinations thereof, or ii) one or more isolated Bifidobacteria or one or more isolated bacteria genetically modified to overexpress human breast milk oligosaccharide metabolizing enzymes, or modified with galT, ureF, ureC or ureE genes.

18. The method of claim 17, wherein the composition is orally administered.

19. The method of claim 17, wherein the composition for the infant is baby formula.

20. The method of claim 17, wherein the pregnant female, infant or child is determined to have or be at risk of compromised immune health using the method of claim 1.
